(19) United States
(12) Patent Application Publication
(10) Pub. No.: US 2024/0093141 A1
(43) Pub. Date: Mar. 21, 2024

(54) STRAINS AND PROCESSES FOR SINGLE CELL PROTEIN OR BIOMASS PRODUCTION

(71) Applicant: Solar Foods Oy, Lappeenranta (EP)

(72) Inventors: Sami HOLMSTRÖM, Espoo (FI); Juha-Pekka PITKÄNEN, Vantaa (FI)

(73) Assignee: Solar Foods Oy, Lappeenranta (FI)

(21) Appl. No.: 17/767,994

(22) PCT Filed: Oct. 22, 2020
(86) PCT No.: PCT/FI2020/050699

§ 371 Date: Apr. 11, 2022

(30) Foreign Application Priority Data
Oct. 29, 2019 (EP) 19205786.7

(51) Int. Cl. C12N 1/20; A23L 33/135; C12N 15/52

(52) U.S. Cl. CPC C12N 1/205; A23L 33/135; C12N 15/52; C12R 2001/01

(57) ABSTRACT

A bacterial strain of the genus Xanthobacter and continuous culture processes for the production of protein or biomass using bacteria of the genus Xanthobacter, said process including supply of gases and minerals to the cells. The present disclosure also relates to the products of these processes and use of these products in e.g. food or feed. Reference is made to the Identification of the Microorganism, having the Identification reference given by the DEPOSITOR of SoF1 and with the Accession number given by the INTERNATIONAL DEPOSITORY AUTHORITY of VTT E-193585. The date of the original deposit is Jun. 11, 2019

### FIELD OF THE INVENTION

The present invention relates to the production of protein and/or other macromolecules using microorganisms. In particular, the invention relates to novel bacterial strains and continuous culture processes for the production of protein or biomass using bacteria wherein gases and minerals are supplied to the cells. The invention also relates to the products of these processes and use of these products in e.g. food or feed.

### BACKGROUND OF THE INVENTION

Growing world population, climate change and shortage of water increasingly pose a threat to traditional agriculture and thus sufficient supply of food and feed. Therefore, alternative sources of organic molecules, such as proteins, are being investigated. A potential alternative is single cell production, i.e. the production of protein and/or other macromolecules using microorganisms.

Chemoautotrophic microorganisms have been described which are able to grow on minimal mineral medium with hydrogen gas as the energy source and carbon dioxide as the only carbon source. For a review of these microorganisms, see e.g. Shively et al. (1998) Annu Rev Microbiol 52:191. Patent application WO2018144965 describes various microorganisms and bioprocesses for converting gaseous substrates into high-protein biomass. Andersen et al. (1979) Biochim Biophys Acta 585:1-11 describes mutant strains of Alcaligenes eutrophus, a hydrogen bacterium that grows readily under heterotrophic and autotrophic conditions. Mutants having altered ribulose-1,5-bisphosphate carboxylase/oxygenase (rubisco) activity were characterised. Ohmiya et al. (2003) J. Biosci. Bioeng. 95:549-561 reviews the application of microbial genes to recalcitrant biomass utilization. Yu Jian et al. (2013) Int J Hydrogen Ener 38:8683-8690 describes carbon dioxide fixation by a hydrogen-oxidizing bacterial isolate. A high energy efficiency of 50% was measured under a moderate oxygen concentration (10 mol %).

However, various chemoautotrophic microorganisms have different properties in terms of growth rate, yield, biomass composition as well as properties related to being used as a food ingredient such as safety in human consumption, taste, smell, mouth-feel, technical and functional properties in cooking, etc. Not every chemoautotrophic microorganism has sufficient growth rate and provides sufficient yield and not every process can realistically be upscaled to an economically viable large-scale process. In order to have sufficient output of functional protein, e.g. for food or feed applications, it is important to find a suitable production organism and a suitable process which can be performed at large scale. This need is addressed by the present invention.

### SUMMARY OF THE INVENTION

In a first main aspect, the invention relates to an isolated bacterial strain VTT-E-193585 or a derivative thereof.

In further aspects, the invention relates to a culture comprising the bacterial strain of the invention or derivative thereof. Furthermore, the invention relates to a process for the production of biomass and/or protein, said process comprising culturing the bacterial strain of the invention or a derivative thereof.

In a further aspect, the invention relates to a process for the production of biomass and/or protein, said process comprising culturing a bacterial strain of the genus Xanthobacter in continuous culture with hydrogen as energy source and an inorganic carbon source, wherein the inorganic carbon source comprises carbon dioxide.

In further main aspects, the invention relates to bulk protein, biomass or non-protein cellular or chemical components obtained or obtainable by the process of the invention, and to a food or feed product obtained or obtainable by a process of the invention.

### BRIEF DESCRIPTION OF THE FIGURES

FIG. 1. Optical density measured at 600 nm (black circles) and optical density probe readings during chemoautotrophic 200-L cultivation of isolated bacterial strain deposited as VTT-E-193585.

FIG. 2. Optical density measured at 600 nm during parallel chemoautotrophic 200-mL cultivations of isolated bacterial strain deposited as VTT-E-193585 on different nitrogen sources.

### DETAILED DESCRIPTION OF THE INVENTION

### Definitions

When used herein, the term “isolated”, e.g. in the context of a strain, means isolated from its natural environment. Preferably, an isolated strain is pure, i.e. free of other strains.

The term “derivative”, when used herein in the context of a strain, refers to a strain which is derived from a reference strain, i.e. generated using the reference strain as starting point. E.g. a genetically-engineered or otherwise mutated or genetically-modified strain is an embodiment of such a derivative. Genetic modifications include point mutations, as well as insertions or deletions, including insertions or deletions of entire loci or fragments thereof. The derivative preferably has fewer than 10 genetic modifications, e.g. fewer than 5, such as 4, 3, 2 or 1 genetic modification(s) compared to the reference strain.

When used herein, the noun “culture” refers to a suspension of viable cells in a liquid medium.

The term “biomass” has its usual meaning in the field of bacterial fermentation and refers to cellular material.

The term “continuous culture”, when used herein, refers to a culturing process wherein fresh media is added continuously to the culture and media with bacterial culture is removed continuously at essentially the same rate.

### ASPECTS AND EMBODIMENTS OF THE INVENTION

In a first main aspect, the invention relates to an isolated bacterial strain VTT-E-193585 or a derivative thereof.

Strain VTT-E-193585 has been isolated from the seashore of the Baltic sea in Naantali, Finland. This organism is able to grow in suitable bioreactor conditions with minimal mineral medium with hydrogen as the energy source and carbon dioxide as the carbon source at limited oxygen conditions. 16S sequencing and Illumina metagenomics sequencing have shown that the strain most likely is a member of the genus Xanthobacter, but is not a known species. The bacterial strain is highly suitable for food and feed applications, because the dried cell powder has a high protein content and contains all the essential amino acids. It also contains more unsaturated than saturated fatty acids and a high level of B-group vitamins. The levels of peptidoglycans and lipopolysaccharides, which may cause allergy or toxicity, are low. A toxicity analysis was performed and no genotoxicity or cytotoxicity was observed for the strain. In addition, the strain is generally sensitive to antibiotics.

Strain VTT-E-193585 (SoF1) has been deposited on Jun. 11th, 2019 in the VTT Culture Collection at the VTT Technical Research Centre of Finland, P.O. Box 1000, FI-02044 VTT, Finland, an International Depositary Authority under the Budapest Treaty. Further information on the characteristics of the strain and methods for culturing the strain are provided in the Examples herein.

In a preferred embodiment, if the strain is a derivative of strain VTT-E-193585, the derivative has retained the ability to grow using hydrogen gas as energy source and carbon dioxide as the only carbon source.

In one embodiment, if the strain is a derivative of strain VTT-E-193585, the derivative comprises the 16S ribosomal RNA set forth in SEQ ID NO:1 or a 16S ribosomal RNA having up to 20 nucleotide differences with SEQ ID NO:1, e.g. 1 to 10, such as 1 to 5, e.g. one, two or three nucleotide differences with SEQ ID NO: 1.

16S ribosomal RNA sequence of strain
VTT-E-193585:
SEQ ID NO: 1
CTTGAGAGTTTGATCCTGGCTCAGAGCGAACGCTGGCGGCAGGCCT
AACACATGCAAGTCGAGCGCCCAGCAATGGGAGCGGCAGACGGGT
GAGTAACGCGTGGGGATGTGCCCAATGGTACGGAATAACCCAGGG
AAACTTGGACTAATACCGTATGAGCCCTTCGGGGGAAAGATTTAT
CGCCATTGGATCAACCCGCGTCTGATTAGCTAGTTGGTGGGGTAA
CGGCCCACCAAGGCGACGATCAGTAGCTGGTCTGAGAGGATGATC
AGCCACACTGGGACTGAGACACGGCCCAGACTCCTACGGGAGGCA
GCAGTGGGGAATATTGGACAATGGGCGCAAGCCTGATCCAGCCAT
GCCGCGTGTGTGATGAAGGCCTTAGGGTTGTAAAGCACTTTCGCC
GGTGAAGATAATGACGGTAACCGGAGAAGAAGCCCCGGCTAACTT
CGTGCCAGCAGCCGCGGTAATACGAAGGGGGCTAGCGTTGCTCGG
AATCACTGGGCGTAAAGCGCACGTAGGCGGATCGTTAAGTCAGGG
GTGAAATCCTGGAGCTCAACTCCAGAACTGCCCTTGATACTGGCG
ACCTTGAGTTCGAGAGAGGTTGGTGGAACTGCGAGTGTAGAGGTG
AAATTCGTAGATATTCGCAAGAACACCAGTGGCGAAGGCGGCCAA
CTGGCTCGATACTGACGCTGAGGTGCGAAAGCGTGGGGAGCAAAC
AGGATTAGATACCCTGGTAGTCCACGCCGTAAACGATGGATGCTA
GCCGTTGGGCAGCTTGCTGTTCAGTGGCGCAGCTAACGCATTAAG
CATCCCGCCTGGGGAGTACGGTCGCAAGATTAAAACTCAAAGGAA
TTGACGGGGGCCCGCACAAGCGGTGGAGCATGTGGTTTAATTCGA
AGCAACGCGCAGAACCTTACCAGCCTTTGACATGGCAGGACGATT
TCCAGAGATGGATCTCTTCCAGCAATGGACCTGCACACAGGTGCT
GCATGGCTGTCGTCAGCTCGTGTCGTGAGATGTTGGGTTAAGTCC
CGCAACGAGCGCAACCCTCGCCTCTAGTTGCCAGCATTCAGTTGG
GCACTCTAGAGGGACTGCCGGTGATAAGCCGAGAGGAAGGTGGGG
ATGACGTCAAGTCCTCATGGCCCTTACGGGCTGGGCTACACACGT
GCTACAATGGTGGTGACAGTGGGATGCGAAAGGGCGACCTCTAGC
AAATCTCCAAAAGCCATCTCAGTTCGGATTGTACTCTGCAACTCG
AGTGCATGAAGTTGGAATCGCTAGTAATCGTGGATCAGCATGCCA
CGGTGAATACGTTCCCGGGCCTTGTACACACCGCCCGTCACACCA
TGGGAGTTGGCTTTACCCGAAGGCGCTGCGCTAACCCGCAAGGGA
GGCAGGCGACCACGGTAGGGTCAGCGACTGGGGTGAAGTCGTAAC
AAGGTAGCCGTAGGGGAACCTGCGGCTGGATCACCTCCTTT.

In a further aspect, the invention relates to a culture comprising the bacterial strain of the invention or derivative thereof. In a preferred embodiment, the volume of the culture is 100 mL or more, e.g. 1 L or more, such as 10 L or more, e.g. 1,000 L or more, such as 10,000 L or more, e.g. 50,000 L or more, such as 100,000 L or more, e.g. 200,000 L or more.

In a further aspect, the invention relates to a process for the production of biomass and/or protein, said process comprising culturing the bacterial strain of the invention or a derivative thereof. In one embodiment, the process is for the production of biomass.

In another embodiment, the process is for the production of protein. In one embodiment, the process comprises culturing the strain in continuous culture with hydrogen as energy source and an inorganic carbon source, wherein the inorganic carbon source comprises carbon dioxide. In a further embodiment, the process is for the production of biomass and comprises culturing the strain in continuous culture with hydrogen as energy source and an inorganic carbon source, wherein the inorganic carbon source comprises carbon dioxide. Various further embodiments of the process are described herein below.

In a further main aspect, the invention relates to a process for the production of biomass and/or protein, said process comprising culturing a bacterial strain of the genus Xanthobacter in continuous culture with hydrogen as energy source and an inorganic carbon source, wherein the inorganic carbon source comprises carbon dioxide. In one embodiment, the process is for the production of biomass. In another embodiment, the process is for the production of protein. Various further embodiments of the process are described herein below.

According to the genome sequence, the strain deposited under number VTT-E-193585 uses most likely Calvin-Benson-Bassham cycle for the carbon fixation where carbon dioxide molecule is connected to 5-carbon chain of ribulose 1,5-bisphosphate forming two molecules of glycerate 3-phosphate. This enables the strain to synthesise all the other organic molecules it requires for growth. Energy from hydrogen comes into the cell most likely through NAD⁺-reducing hydrogenases and/or NiFeSe-hydrogenases. In essence that is a redox reaction where hydrogen (H₂) is oxidized to H⁺ and NAD⁺is reduced to NADH. In addition to ATP, NADH is one of the main energy carriers inside living organisms. Alternatively, some other energy equivalent is reduced by another hydrogenase enzyme using H₂. The Calvin-Benson-Bassham cycle requires energy in the form of ATP and NADH/NADPH in order to fix CO₂. The strain most likely generates ATP through oxidative phosphorylation, which consists of four protein complexes generating a proton gradient across a membrane. The proton gradient is generated using mainly energy from NADH. The proton gradient drives the ATP synthase complex generating ATP. According to the genome sequence, the strain has a bacterial F-type ATP synthase.

It is to be understood, when it is specified that the process comprises culturing the strain with an inorganic carbon source, that the inorganic carbon source is the main carbon source in the culture. Thus, there may be minor amounts of organic carbon sources present in the culture, but the main metabolism and growth of the culture is based on the utilisation of the inorganic carbon source, preferably carbon dioxide, as carbon source. Preferably the proportion of the carbon supplied to the culture that is organic is less than 5%, such as less than 1%, e.g. less than 0.1% of all carbon supplied to the culture during the process. Preferably, no organic carbon sources are supplied to the process.

Similarly, it is to be understood, when it is specified that the process comprises culturing the strain with hydrogen (H₂) as energy source, that hydrogen is the main energy source in the culture. Thus, there may be other minor energy sources present in the culture such as ammonia, which may be supplied as nitrogen source, or minor amounts of organic compounds, but the main metabolism and growth of the culture is based on the utilisation of hydrogen as energy source. In the overall process hydrogen is preferably produced by water electrolysis; i.e. by splitting water with electricity to hydrogen and oxygen gases. Thus, the hydrogen and oxygen gases are provided to the bioreactor from an electrolyser nearby. Alternatively, electrodes may be placed inside the bioreactor to produce hydrogen and oxygen in the bioreactor rather than in a separate electrolyser.

The inorganic carbon source comprising carbon dioxide may comprise other inorganic carbon sources, such as e.g. carbon monoxide. In one embodiment, only carbon sources in gaseous form are provided to the culture. In a preferred embodiment, carbon dioxide is the only inorganic carbon source, and indeed the only carbon source, provided to the culture. In one embodiment, only gases and minerals are provided to the culture and the level of carbon dioxide in the gas provided is between 10% and 50%, e.g. between 15% and 45%, such as between 20% and 40%, e.g. between 25% and 35%, such as between 26% and 30%.

In another embodiment, gases and minerals are provided to the culture and the level of hydrogen (H₂) in the gas provided is between 30% and 80%, e.g. between 35% and 75%, such as between 40% and 70%, e.g. between 45% and 65%, such as between 50% and 60%.

In another embodiment, gases and minerals are provided to the culture and the level of oxygen (O₂) in the gas provided is between 10% and 25%, e.g. between 15% and 20%, such as between 16% and 18%. In another embodiment, the level of oxygen provided is such that the level of dissolved oxygen in the culture is maintained at between 5% and 10%.

In a preferred embodiment, only gases and minerals are provided to the culture and the gas provided comprising H₂, CO₂ and O₂, wherein the percentage of H₂ is between 40% and 70%, the percentage of CO₂ is between 18% and 28% and the percentage of O₂ is between 12% and 22%.

Typically, the process of the invention includes the addition of a nitrogen source. The nitrogen source may for example be provided in the form of ammonium hydroxide, an ammonium salt, such as ammonium sulphate or ammonium chloride, ammonia, urea or nitrate, e.g. potassium nitrate. In other embodiments, nitrogen gas (N₂) is provided as nitrogen source. In a preferred embodiment, the nitrogen source is ammonium hydroxide or an ammonium salt, such as ammonium sulphate.

In one embodiment, the nitrogen source provided is ammonium hydroxide at a concentration of between 100 mg/L and 10 g/L, such as between 250 mg/L and 4 g/L, e.g. between 0.5 g/L and 2 g/L, such as between 0.75 g/L and 1.5 g/L.

Typically, the process of the invention includes the addition of minerals, such as minerals containing ammonium, phosphate, potassium, sodium, vanadium, iron, sulphate, magnesium, calcium, molybdenum, manganese, boron, zinc, cobalt, selenium, iodine, copper and/or nickel. Suitable mineral media are well-known art, and have e.g. been described in Thermophilic Bacteria, CRC Press, Boca Raton, FL, Jacob K. Kristjansson, ed., 1992, for example on page 87, Table 4.

In one embodiment, the minerals added include one or more of the following: ammonia, ammonium (e.g., ammonium chloride (NH₄Cl), ammonium sulphate ((NH₄)₂SO₄)), nitrate (e.g., potassium nitrate (KNO₃)), urea or an organic nitrogen source; phosphate (e.g., disodium phosphate (Na₂HPO₄), potassium phosphate (KH₂PO₄), phosphoric acid (H₃PO₄), potassium dithiophosphate (K₃PS₂O₂), potassium orthophosphate (K₃PO₄), disodium phosphate (Na₂HPO₄·2H₂O) dipotassium phosphate (K₂HPO₄) or monopotassium phosphate (KH₂PO₄); sulphate; yeast extract; chelated iron (chelated e.g. with EDTA or citric acid); potassium (e.g., potassium phosphate (KH₂PO₄), potassium nitrate (KNO₃), potassium iodide (KI), potassium bromide (KBr)); and other inorganic salts, minerals, and trace nutrients (e.g., sodium chloride (NaCl), magnesium sulphate (MgSO₄·7H₂O) or magnesium chloride (MgCl₂), calcium chloride (CaCl₂), calcium sulphate (CaSO₄) or calcium carbonate (CaCO₃), manganese sulphate (MnSO₄·7H₂O) or manganese chloride (MnCl₂), ferric chloride (FeCl₂), ferrous sulphate (FeSO₄ 7H₂O) or ferrous chloride (FeCl₂ 4H₂O), sodium bicarbonate (NaHCO₃) or sodium carbonate (Na₂CO₃), zinc sulphate (ZnSO₄) or zinc chloride (ZnCl₂), ammonium molybdate (NH₄MoO₄) or sodium molybdate (Na₂MoO₄·2H₂O), cuprous sulphate (CuSO4) or copper chloride (CuCl₂·2H₂O), cobalt chloride (CoCl₂·6H₂O) or cobalt sulphate (CoSO₄), aluminium chloride (AlCl₃·6H₂O), lithium chloride (LiCl), boric acid (H₃BO₃), nickel chloride NiCl₂·6H₂O) or nickel sulphate (NiSO₄), tin chloride (SnCl₂·H₂O), barium chloride (BaCl₂·2H₂O), copper selenate (CuSeO₄ 5H₂O), sodium selenate (Na₂SeO₄) or sodium selenite (Na₂SeO₃), sodium metavanadate (NaVO₃), chromium salts).

In a preferred embodiment, the process of the invention includes the addition of one, more or all of: NH₄OH, KH₂PO₄, Na₂HPO₄·2H₂O, NaVO₃·H₂O, FeSO₄x7H₂O, MgSO₄-7H₂O, CaSO₄, Na₂MoO₄-2H₂O, MnSO₄-7H₂O, ZnSO₄-7H₂O, H₃BO₃, CoSO₄, CuSO₄, NiSO₄.

In one embodiment, the medium provided to the cells comprises less than 1 g/L of chloride salts, such as less than 0.25 g/L of chloride salts, e.g. less than 0.1 g/L of chloride salts, such as less than 0.025 g/L of chloride salts, e.g. less than 0.01 g/L of chloride. In one embodiment, no chloride salts are supplied to the culture.

In another embodiment, no vitamins are supplied during the process, i.e. the media provided to the culture does not contain vitamins.

In another embodiment, no amino acids are supplied during the process, i.e. the media provided to the culture does not contain amino acids.

In another embodiment, no organic compounds are supplied during the process, i.e. the media provided to the culture does not contain any organic compounds.

In certain embodiments, the pH of the bacterial culture is controlled at a certain level. In certain embodiments, pH is controlled within an optimal range for bacterial maintenance and/or growth and/or production of organic compounds. In one embodiment, the pH in the culture is maintained between 5.5 and 8.0, e.g. between 6.5 and 7.0, such as at 6.8.

In certain embodiments, the temperature of the bacterial culture is controlled. In certain embodiments, temperature is controlled within an optimal range for bacterial maintenance and/or growth and/or production of organic compounds. In one embodiment, the culture is grown at a temperature between 25° C. and 40° C., e.g. between 28° C. and 32° C., such as at 30° C.

Typically, the process of the invention is carried out in a bioreactor. A bioreactor is utilized for the cultivation of cells, which may be maintained at particular phases in their growth curve. The use of bioreactors is advantageous in many ways for cultivating chemoautotrophic growth. Generally, the control of growth conditions, including control of dissolved carbon dioxide, oxygen, and other gases such as hydrogen, as well as other dissolved nutrients, trace elements, temperature and pH, is facilitated in a bioreactor. Nutrient media, as well as gases, can be added to the bioreactor as either a batch addition, or periodically, or in response to a detected depletion or programmed set point, or continuously while the period the culture is grown and/or maintained. In a continuous culture process, nutrient media, as well as gases, are added to the bioreactor continuously. Furthermore, bacteria-containing medium is being removed from the bioreactor continuously.

In a preferred embodiment, the volume of the bacterial culture is 100 mL or more, such as 1 L or more, e.g. 10 L or more, such as 100 L or more, e.g. 1,000 L or more, such as 10,000 L or more, e.g. 50,000 L or more, such as 100,000 L or more, e.g. 200,000 L or more.

In one embodiment, the productivity of the culture is more than 0.1 g cell dry weight per liter per hour, such as more than 0.2, e.g. more than 0.3, such as more than 0.4, e.g. more than 0.5, such as more than 0.6, e.g. more than 0.7, such as more than 0.8, e.g. more than 0.9, such as more than 1 g per liter per hour.

Bacteria can be inoculated directly from a cell bank, or via a seed culture at a smaller scale. Preferably, supply of fresh media to the culture and removal of used up media with bacteria is occurring at the same rate, such that the volume in the bioreactor remains the same.

In one embodiment, after an initial phase of reaching a suitable cell density, the bacteria grow at steady state or pseudo steady state, remaining continuously in their log phase, at an OD600 above 5, such as above 10, e.g. above 20, such as between 50 and 200, e.g. between 50 and 100.

In one embodiment of the process of the invention, the bacterial strain has a growth rate of 0.04-0.12 h⁻¹.

In another embodiment of the process of the invention, the liquid feed rate in the continuous phase is 50-80% of the growth rate.

Xanthobacter is a genus of Gram-negative bacteria from the Xanthobacteraceae family.

In one embodiment, the Xanthobacter strain used in the process of the invention is a 25 strain which uses the Calvin Benson Bassham pathway to convert carbon dioxide into organic compounds, e.g. glucose, essential for living organisms.

In one embodiment, the Xanthobacter strain used in the process of the invention is a strain which uses NiFeSe-hydrogenases for converting hydrogen (H₂) into cellular 30 energy equivalents.

In one embodiment, the Xanthobacter strain used in the process of the invention is a strain which uses NAD⁺-reducing hydrogenases for converting hydrogen (H₂) into cellular energy equivalents.

In one embodiment, the Xanthobacter strain used in the process of the invention capable of nitrogen fixation.

In one embodiment, the bacterial strain used in the process of the invention is selected from the group consisting of: X. agilis, X. aminoxidans, X. autotrophicus, X. flavus, X. tagetidis, X. viscosus, Xanthobacter sp. 126, Xanthobacter sp. 91 and strain VTT-E-193585.

In a preferred embodiment, the bacterial strain used in the process of the invention is VTT-E-193585 or X. tagetidis. Most preferably the strain used in the process of the invention is VTT-E-193585.

In another embodiment, the bacterial strain used in the process of the invention comprises the 16S ribosomal RNA set forth in SEQ ID NO:1 or a 16S ribosomal RNA having up to 20 nucleotide differences with SEQ ID NO:1, e.g. 1 to 10, such as 1 to 5, e.g. one, two or three nucleotide differences with SEQ ID NO: 1.

In another embodiment, the bacterial strain used in the process of the invention comprises a gene encoding a ribulose-1,5-bisphosphate carboxylase/oxygenase (rubisco) large chain having the sequence set forth in SEQ ID NO:3 or a sequence having more than more than 93% identity, e.g. more than 95% identity, such as more than 96% identity, e.g. more than 97% identity, such as more than 98% identity, e.g. more than 99% sequence identity to the sequence set forth in SEQ ID NO:3.

In another embodiment, the bacterial strain used in the process of the invention comprises a gene encoding a ribulose-1,5-bisphosphate carboxylase/oxygenase (rubisco) small chain having the sequence set forth in SEQ ID NO:5 or a sequence having more than 83% sequence identity, e.g. more than 86%, identity such as more than 90% identity, e.g. more than 95% identity, such as more than 96% identity, e.g. more than 97% identity, such as more than 98% identity, e.g. more than 99% sequence identity to the sequence set forth in SEQ ID NO:5.

SEQ ID NO: 2:
Nucleotide sequence of Ribulose
bisphosphate carboxylase large chain:
ATGGGTGCCGAAGCAACCGTCGGGCAGATCACGGACGCCAAGAAG
AGATACGCCGCCGGCGTGCTGAAGTACGCCCAGATGGGCTACTGG
AACGGCGACTACGTTCCCAAGGACACCGACCTCCTGGCGGTGTTC
CGCATCACCCCCCAGGCGGGCGTGGACCCGGTGGAAGCCGCCGCG
GCGGTCGCCGGCGAAAGCTCCACCGCTACCTGGACCGTGGTGTGG
ACCGACCGGCTCACCGCCGCCGACGTCTACCGCGCCAAGGCCTAC
AAGGTGGAGCCGGTGCCGGGCCAGGAAGGCCAGTATTTCTGCTAC
ATCGCCTATGATCTCGATTTGTTCGAGGAAGGCTCCATCGCCAAC
CTCACGGCGTCGATCATCGGCAACGTCTTCTCCTTCAAGCCGCTG
AAGGCGGCGCGGCTGGAGGACATGCGGCTTCCCGTCGCCTATGTG
AAGACCTTCCGCGGCCCGCCCACCGGCATCGTGGTCGAGCGCGAG
CGCCTGGACAAGTTCGGCCGCCCCCTTCTGGGCGCCACCACCAAG
CCGAAGCTTGGCCTCTCGGGCAAGAATTACGGCCGCGTGGTCTAT
GAGGCCCTCAAGGGCGGCCTCGACTTCGTGAAGGACGACGAGAAC
ATCAACTCGCAGCCCTTCATGCACTGGCGCGATCGCTTCCTCTAT
TGCATGGAGGCCGTCAACAAGGCCCAGGCCGAGACCGGCGAGGTG
AAGGGGCACTATCTCAACATCACCGCCGGGACCATGGAGGAGATG
TACCGCCGCGCCGAGTTCGCCAAGGAACTGGGCTCCGTGGTGGTG
ATGGTGGATCTCATCATCGGCTGGACCGCCATCCAGTCCATGTCC
AACTGGTGCCGCGAGAACGACATGATCCTGCACATGCACCGTGCG
GGCCATGGCACCTACACGCGCCAGAAGAGCCACGGCGTCTCCTTC
CGCGTCATCGCCAAGTGGCTGCGGCTCGCCGGCGTCGACCACCTG
CACACCGGCACCGCCGTGGGCAAGCTGGAAGGCGACCCCATGACC
GTGCAGGGCTTCTACAATGTCTGCCGCGAGACGACGACGCAGCAG
GACCTCACCCGCGGCCTGTTCTTCGAGCAGGACTGGGGCGGCATC
CGCAAGGTGATGCCGGTGGCCTCCGGCGGCATCCATGCGGGCCAG
ATGCACCAGCTCATCGACCTGTTCGGCGAGGACGTGGTGCTCCAG
TTCGGCGGCGGCACCATCGGCCACCCGGACGGCATCCAGGCCGGC
GCCACCGCCAACCGCGTGGCGCTGGAAACCATGATCCTCGCCCGC
AACGAGGGCCGCGACATCAGGAACGAGGGCCCGGAAATCCTGGTG
GAAGCCGCCAAATGGTGCCGTCCGCTGCGCGCGGCGCTCGATACC
TGGGGCGAGGTGACCTTCAACTACGCCTCCACCGACACGTCCGAT
TACGTGCCCACCGCGTCCGTCGCCTGA
SEQ ID NO: 3:
Amino acid sequence of Ribulose
bisphosphate carboxylase large chain
MGAEATVGQITDAKKRYAAGVLKYAQMGYWNGDYVPKDTDLLAVF
RITPQAGVDPVEAAAAVAGESSTATWTVVWTDRLTAADVYRAKAY
KVEPVPGQEGQYFCYIAYDLDLFEEGSIANLTASIIGNVFSFKPL
KAARLEDMRLPVAYVKTFRGPPTGIVVERERLDKFGRPLLGATTK
PKLGLSGKNYGRVVYEALKGGLDFVKDDENINSQPFMHWRDRFLY
CMEAVNKAQAETGEVKGHYLNITAGTMEEMYRRAEFAKELGSVVV
MVDLIIGWTAIQSMSNWCRENDMILHMHRAGHGTYTRQKSHGVSF
RVIAKWLRLAGVDHLHTGTAVGKLEGDPMTVQGFYNVCRETTTQQ
DLTRGLFFEQDWGGIRKVMPVASGGIHAGQMHQLIDLFGEDVVLQ
FGGGTIGHPDGIQAGATANRVALETMILARNEGRDIRNEGPEILV
EAAKWCRPLRAALDTWGEVTFNYASTDTSDYVPTASVA
SEQ ID NO: 4:
Nucleotide sequence of Ribulose
bisphosphate carboxylase small chain:
ATGCGCATCACCCAAGGCTCCTTCTCCTTCCTGCCGGACCTCACC
GACACGCAGATCAAGGCCCAGGTGCAATATTGCCTGGACCAGGGC
TGGGCGGTCTCGGTGGAGCACACCGACGATCCCCACCCGCGCAAC
ACCTATTGGGAGATGTGGGGCCCGCCCATGTTCGATCTGCGCGAC
GCGGCCGGCGTCTTCGGCGAGATCGAAGCCTGCCGGGCCGCCAAT
CCCGAGCATTATGTGCGGGTGAACGCCTTCGATTCCAGCCGCGGA
TGGGAGACGATCCGCCTGTCCTTCATCGTTCAGCGGCCCACCGTG
GAAGAGGGCTTCCGCCTCGACCGCACCGAAGGCAAGGGCCGCAAC
CAGAGCTACGCCATGCGCTACCGGGCGCAGTTCGCGCCGCGCTGA
SEQ ID NO: 5:
Amino acid sequence of Ribulose
bisphosphate carboxylase small chain:
MRITQGSFSFLPDLTDTQIKAQVQYCLDQGWAVSVEHTDDPHPRN
TYWEMWGPPMFDLRDAAGVFGEIEACRAANPEHYVRVNAFDSSRG
WETIRLSFIVQRPTVEEGFRLDRTEGKGRNQSYAMRYRAQFAPR

In another embodiment, the bacterial strain used in the process of the invention comprises a gene encoding a NAD⁺-reducing hydrogenase HoxS subunit alpha having the sequence set forth in SEQ ID NO:7 or a sequence having more than 70% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:7.

In another embodiment, the bacterial strain used in the process of the invention comprises a gene encoding a NAD⁺-reducing hydrogenase HoxS subunit beta having the sequence set forth in SEQ ID NO:9 or a sequence having more than 77% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:9.

In another embodiment, the bacterial strain used in the process of the invention comprises a gene encoding a NAD⁺-reducing hydrogenase HoxS subunit gamma having the sequence set forth in SEQ ID NO: 11 or a sequence having more than 70% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:11.

In another embodiment, the bacterial strain used in the process of the invention comprises a gene encoding a NAD⁺-reducing hydrogenase HoxS subunit delta having the sequence set forth in SEQ ID NO: 13 or a sequence having more than 79% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:13.

SEQ ID NO: 6:
Nucleotide sequence of NAD⁺-
reducing hydrogenase HoxS subunit alpha:
ATGATGCCATCTGAGCCGCACGGCGCGGGCATGCCGCCCCCACGG
GAAGCGGCCGCGGTTCCCACCCCCCAGGAGGTGAGCGCGGTGGTG
GCCGAGGTGGTCGCGGATGCCGTGGCATCGGTGGGCGGCGCACGC
ACCCGGCTCATGGACATCGTCCAGCTGGCCCAGCAGCGTCTCGGC
CATCTCTCCGAAGAGACCATGGCGGCCATTGCCGCGCGGCTCGCC
ATTCCGCCGGTGGAAGTGGCGGACATGGTGTCCTTCTACGCCTTC
CTGAACCGCGCGCCCAAGGGCCGCTACCACATCCGCCTGTCGCGC
AGCCCCATCTCGCTGATGAAGGGCGCCGAGGCGGTGGCTGCCGCC
TTCTGCCAGATCCTCGGCATCGCCATGGGCGAGACCTCGCAGGAT
GGCGACTTCACCCTGGAATGGACCAACGACATCGGCATGGCCGAC
CAGGAGCCGGCCGCCCTCGTCAACGGCACGGTGATGACGCAGCTC
GCGCCCGGCGATGCGGCCATCATCGTCGGCCGGCTGCGGGCCCAT
CACGCGCCCAATGCCCTGCCGCTGTTCCCTGGAGCCGGCGTGGCC
GGCTCCGGCCTGCCCCATGCCCGGATCCGCCCCAGCCTGGTGATG
CCGGGACAGCTTCTGTTCCGCGAGGACCACACGACGCCGGGCGCC
GGCATCAAGGCGGCACTCGCCCTCACCCCGGACGAAGTGGTGCAG
AAGGTCTCCGCCGCGCGCCTGCGCGGGGGGGGTGGCGCCGGCTTT
CCCACCGGTCTCAAATGGAAGCTCTGCCGCCAGTCGCCCGCCACC
ACCCGCCATGTGATCTGCAATGCGGACGAGGGCGAGCCCGGCACC
TTCAAGGATCGCGTGCTGCTCACGCAGGCGCCGCACCTCATGTTC
GACGGCATGACCATCGCCGGCTACGCCTTGGGGGCGCGGGAGGGC
GTGGTCTATCTGCGCGGCGAGTACGCCTATCTGTGGGAGCCTCTG
CATGCGGTCCTGCGCGAGCGCTATGGGCTCGGGCTCGCCGGCGCG
AACATCCTGGGACACGCGGGCTTCGACTTCGACATCCGCATCCAG
CTGGGCGCCGGCGCCTATATCTGCGGCGAGGAATCCGCGCTGGTG
GAATCGCTGGAAGGCAAGCGCGGCTCGCCCCGCGACCGCCCCCCC
TTCCCCACCGTGCGCGGCCATCTCCAGCAGCCCACCGCCGTGGAC
AATGTGGAGACCTTCGCCTGCGCCGCCCGCATCCTGGAGGATGGC
GTGGAGGCGTTCGCGGGCATCGGCACGCCCGAATCCGCCGGCACG
AAGCTCCTCTCGGTGTCGGGCGATTGCCCGCGCCCCGGCGTGTAT
GAGGTGCCCTTCGGCCTCACGGTGAACGCGCTGCTCGACCTTGTC
GGCGCGCCGGACGCCGCCTTCGTGCAGATGGGTGGGCCGTCCGGC
CAATGCGTGGCGCCGAAGGATTACGGCCGCCGCATCGCCTTCGAG
GACCTGCCCACCGGCGGCTCGGTGATGGTGTTCGGCCCGGGGCGC
GACGTGCTCGCCATGGTGCGCGAGTTCGCGGATTTCTTCGCCGGC
GAATCCTGCGGCTGGTGCACGCCCTGCCGGGTGGGCACCACCTTG
CTCAAGGAAGAGCTGGACAAGCTCCTCGCCAACCGCGCCACCCTC
GCCGACATCCGCGCGCTGGAGACCCTGGCCACGACCGTCTCCCGC
ACCAGCCGCTGCGGCCTCGGCCAGACGGCGCCCAACCCCATCCTT
TCCACCATGCGCAACCTGCCGGAAGCCTATGAGGCGAGGCTGAGG
CCCGAAGACTTCCTGCCCTGGGCCTCGCTCGACGAGGCGCTGAAG
CCCGCCATCGTCATCCAGGGCCGCGCGCCCGTGCCGGAGGAAGAG
GCATGA
SEQ ID NO: 7:
Amino acid sequence of NAD⁺-
reducing hydrogenase HoxS subunit alpha:
MMPSEPHGAGMPPPREAAAVPTPQEVSAVVAEVVADAVASVGGAR
TRLMDIVQLAQQRLGHLSEETMAAIAARLAIPPVEVADMVSFYAF
LNRAPKGRYHIRLSRSPISLMKGAEAVAAAFCQILGIAMGETSQD
GDFTLEWTNDIGMADQEPAALVNGTVMTQLAPGDAAIIVGRLRAH
HAPNALPLFPGAGVAGSGLPHARIRPSLVMPGQLLFREDHTTPGA
GIKAALALTPDEVVQKVSAARLRGRGGAGFPTGLKWKLCRQSPAT
TRHVICNADEGEPGTFKDRVLLTQAPHLMFDGMTIAGYALGAREG
VVYLRGEYAYLWEPLHAVLRERYGLGLAGANILGHAGFDFDIRIQ
LGAGAYICGEESALVESLEGKRGSPRDRPPFPTVRGHLQQPTAVD
NVETFACAARILEDGVEAFAGIGTPESAGTKLLSVSGDCPRPGVY
EVPFGLTVNALLDLVGAPDAAFVQMGGPSGQCVAPKDYGRRIAFE
DLPTGGSVMVFGPGRDVLAMVREFADFFAGESCGWCTPCRVGTTL
LKEELDKLLANRATLADIRALETLATTVSRTSRCGLGQTAPNPIL
STMRNLPEAYEARLRPEDFLPWASLDEALKPAIVIQGRAPVPEEE
A
SEQ ID NO: 8:
Nucleotide sequence of NAD⁺-
reducing hydrogenase HoxS subunit beta:
ATGAGCCGGGGATCCCCCGATGCCGGGAAAGACCGCACCATGAGC
GCCACCGACGGCACCACCGCCCCCCGCAAGATCGTCATCGATCCG
GTGACCCGCGTGGAGGGCCACGGCAAGGTCACCATCCGCCTGGAT
GAAGCCGGCGCGGTGGAGGATGCGCGTTTCCACATCGTGGAGTTC
CGCGGCTTCGAGCGGTTCATCCAGGGCCGGATGTACTGGGAAGTG
CCCCTTATCATCCAGCGGCTGTGCGGCATCTGCCCGGTGAGCCAC
CATCTGGCGGCGGCGAAAGCCATGGACCAGGTGGCGGGCGTGGAC
CGCGTACCGCCCACCGCCGAGAAACTGCGCCGGCTGATGCATTAT
GGGCAGGTGCTGCAATCCAACGCTTTGCACATCTTCCACCTCGCC
TCGCCCGACCTCCTGTTCGGCTTCGACGCGCCGGCCGAGCAGCGC
AACATCATCGCCGTGCTCCAGCGTTATCCGGAGATCGGCAAATGG
GCGATCTTCATCAGGAAGTTCGGCCAGGAGGTCATCAAGGCCACC
GGCGGGCGCAAGATCCATCCCACCAGCGCCATTCCCGGCGGGGTC
AACCAGAACCTCGCCGTGGAGGACCGCGACGCCCTGCGCGCCAAG
GTGGGCGAGATCATCAGCTGGTGCATGGCGGCGCTGGACCATCAC
AAGGCCTATGTGGCGGAAAACCGGGCGCTGCATGACAGCTTCGCC
GCCTTCCCCTCCGCCTTCATGAGCCTCGTGGGGCCGGATGGCGGC
ATGGACCTTTATGACGGCACCCTGCGGGTGATCGATGCCGAGGGC
GCCCCCCTCATCGAAGGCGCGCCGCCCGCCTCCTACCGCGACCAC
CTCATCGAGGAGGTGCGGCCCTGGAGCTATCTGAAATTCCCCCAT
CTGCGCGCCTTCGGCCGCGACGATGGCTGGTATCGGGTCGGCCCC
CTCGCCCAGGTCAATTGCGCCGCGTCCATCGACACGCCCCGCGCC
GAGGCGGCCCGGCGGGACTTCATGGCCGAGGGGGGGGCAAGCCGG
TGCATGCCACCCTCGCTTATCACTGGGCGCGGCTCATCGTGCTGG
TCCATTGCGCGGAGAAGATCGAACAGCTGCTGTTCGACGACGACC
TGCAAGGCTGCGATCTGCGTGCGGAGGGCACCCGGCGCGGGGAAG
GCGTCGCCTGGATCGAGGCGCCGCGCGGCACCCTCATCCACCATT
ACGAGGTGGACGAGAACGACCAGGTGCGCCGCGCCAACCTCATCG
TCTCCACCACCCACAATAACGAGGCCATGAACCGCGCCGTGCGGC
AGGTGGCGAAGACGGACCTTTCCGGTCGCGAGATCACCGAAGGGC
TGCTGAACCATATCGAGGTGGCCATCCGCGCCTTCGACCCCTGCC
TGTCCTGCGCCACCCATGCGCTGGGCCAGATGCCGCTGATCGTGA
CGCTTGAAGATGCCTCCGGCGCAGAGATCGCCCGCGGAGTGAAGG
AATGA
SEQ ID NO: 9:
Amino acid sequence of NAD⁺-
reducing hydrogenase HoxS subunit beta:
MSRGSPDAGKDRTMSATDGTTAPRKIVIDPVTRVEGHGKVTIRLD
EAGAVEDARFHIVEFRGFERFIQGRMYWEVPLIIQRLCGICPVSH
HLAAAKAMDQVAGVDRVPPTAEKLRRLMHYGQVLQSNALHIFHLA
SPDLLFGFDAPAEQRNIIAVLQRYPEIGKWAIFIRKFGQEVIKAT
GGRKIHPTSAIPGGVNQNLAVEDRDALRAKVGEIISWCMAALDHH
KAYVAENRALHDSFAAFPSAFMSLVGPDGGMDLYDGTLRVIDAEG
APLIEGAPPASYRDHLIEEVRPWSYLKFPHLRAFGRDDGWYRVGP
LAQVNCAASIDTPRAEAARRDFMAEGGGKPVHATLAYHWARLIVL
VHCAEKIEQLLFDDDLQGCDLRAEGTRRGEGVAWIEAPRGTLIHH
YEVDENDQVRRANLIVSTTHNNEAMNRAVRQVAKTDLSGREITEG
LLNHIEVAIRAFDPCLSCATHALGQMPLIVTLEDASGAEIARGVK
E
SEQ ID NO: 10:
Nucleotide sequence of NAD⁺-
reducing hydrogenase HoxS subunit gamma:
ATGAGCGAGACCCCCTTCACCTTTACCGTGGACGGCATCGCGGTC
CCGGCCACCCCCGGCCAGAGCGTCATCGAGGCGTGCGATGCGGGG
GCATCTATATCCCGCGCCTGTGCCACCACCCGGACCTGCCGCCGG
CGGGCCATTGCCGGGTGTGCACCTGCATCATCGACGGGCGGCCGG
CCAGCGCCTGCACCATGCCCGCCGCCAGGGGCATGGTGGTGGAGA
ACGAGACGCCCGCTTTGCTGGCGGAGCGGCGCACGCTGATCGAGA
TGCTGTTCGCGGAAGGCAACCATTTCTGCCAGTTCTGCGAGGCGA
GCGGCGATTGCGAATTGCAGGCGCTGGGCTACCTGTTCGGCATGG
TGGCCCCGCCCTTCCCCCATCTGTGGCCGAAGCGGCCGGTGGATG
CCAGCCATCCGGATATCTATATCGACCACAATCGCTGCATCCTGT
GCTCGCGCTGCGTGCGCGCCTCGCGCACCCTGGACGGCAAGTCCG
TGTTCGGCTTCGAGGGGCGCGGCATCGAGATGCATCTGGCGGTGA
CCGGCGGGCACCTGGACGACAGCGCCATCGCCGCCGCCGACAGGG
CGGTTGAGATGTGCCCGGTGGGCTGCATCGTCCTCAAGCGCACCG
GCTACCGCACGCCCTATGGCCGGCGGCGCTACGACGCCGCGCCCA
TCGGCTCCGACATCACCGCCCGGCGCGGCGGCGCGAAGGACTGA
SEQ ID NO: 11:
Amino acid sequence of NAD⁺-
reducing hydrogenase HoxS subunit gamma:
MSETPFTFTVDGIAVPATPGQSVIEACDAAGIYIPRLCHHPDLPP
AGHCRVCTCIIDGRPASACTMPAARGMVVENETPALLAERRTLIE
MLFAEGNHFCQFCEASGDCELQALGYLFGMVAPPFPHLWPKRPVD
ASHPDIYIDHNRCILCSRCVRASRTLDGKSVFGFEGRGIEMHLAV
TGGHLDDSAIAAADRAVEMCPVGCIVLKRTGYRTPYGRRRYDAAP
IGSDITARRGGAKD
SEQ ID NO: 12:
Nucleotide sequence of NAD⁺-
reducing hydrogenase HoxS subunit delta:
ATGGCCAAGCCCAAACTCGCCACCTGCGCGCTGGCCGGCTGCTTC
GGCTGCCACATGTCCTTCCTGGACATGGACGAGCGCATCGTCGAG
CTCATCGACCTGGTGGACCTCGACGTCTCGCCCCTCGACGACAAG
AAAAACTTCACCGGCATGGTGGAAATCGGCCTGGTGGAAGGCGGC
TGCGCCGACGAGCGCCATGTGAAGGTGCTGCGCGAGTTCCGCGAG
AAATCCCGCATCCTGGTGGCGGTGGGCGCCTGCGCCATCACCGGC
GGCATCCCGGCATTGCGCAACCTCGCCGGCCTCGACGAATGCCTG
AGGGAAGCCTACCTCACCGGCCCCACGGTGGAAGGCGGCGGGCTC
ATTCCCAACGACCCGGAGCTGCCGCTGCTGCTGGACAAGGTCTAT
CCGGTGCAGGACTTCGTGAAGATCGACCATTTCCTGCCCGGCTGC
CCGCCCTCGGCCGACGCCATCTGGGCGGCTCTGAAGGCGCTGCTG
ACCGGCACCGAGCCGCATCTGCCCTACCCGCTTTTCAAGTACGAA
TGA
SEQ ID NO: 13:
Amino acid sequence of NAD⁺-
reducing hydrogenase HoxS subunit delta:
MAKPKLATCALAGCFGCHMSFLDMDERIVELIDLVDLDVSPLDDK
KNFTGMVEIGLVEGGCADERHVKVLREFREKSRILVAVGACAITG
GIPALRNLAGLDECLREAYLTGPTVEGGGLIPNDPELPLLLDKVY
PVQDFVKIDHFLPGCPPSADAIWAALKALLTGTEPHLPYPLFKYE

In another embodiment, the bacterial strain used in the process of the invention comprises a gene encoding a NiFeSe hydrogenase large subunit having the sequence set forth in SEQ ID NO:15 or a sequence having more than 84% sequence identity, e.g. more than 90% identity, such as more than 95% identity, e.g. more than 96% identity, such as more than 97% identity, e.g. more than 98%, such as more than 99% sequence identity to the sequence set forth in SEQ ID NO:15.

In another embodiment, the bacterial strain used in the process of the invention comprises a gene encoding a NiFeSe hydrogenase small subunit having the sequence set forth in SEQ ID NO: 17 or a sequence having more than 90% identity, such as more than 95% identity, e.g. more than 96% identity, such as more than 97% identity, e.g. more than 98%, such as more than 99% sequence identity to the sequence set forth in SEQ ID NO:17.

SEQ ID NO: 14:
Nucleotide sequence of Periplasmic [NiFeSe]
hydrogenase large subunit:
TCCAGACCCGGGCAACATTGCTCCATGTGCTGGGCACCCTGGCCG
GCCGCTGGCCCCATACCCTCGCGCTCCAGCCCGGCGGGGTGACCC
GAAGCGCCGACCAGCACGACCGCATGCGCCTGCTCGCGACGCTGA
AGGCGGTGCGGGGGGCGCTGGAAGAGACCTTGTTCGGCGCGCCTT
TGGAAGAGGTGGCGGCCCTGGACGGCGCCGCCGCCGTGGAGGCCT
GGCGCGCCAACGGCCCGGAAGGGGATTTCCGCCTGTTCCTGGAGA
TCGCCGCCGACCTGGAGCTGGACCGGCTCGGCCGCGCGCACGACC
GCTTTCTCTCCTTCGGCGCCTACGCCCAGGACGAGGGGCGCCTTT
ATGGCGCCGGCACCTTCGAGGCCGGGACGGGGGAGGGCTCGATCC
CAACGCCATCACCGAGGACCACGCCTTCGCCCGCATGGAGGACCG
CGCGGCGCCCCATGCGCCCTTTGACGGCTCCACCTTCCCCGATGC
CGACGACACCGAGGGCTACACCTGGTGCAAGGCGCCGCGCCTTGC
CGGCCTGCCCTTCGAGACCGGCGCCTTCGCCCGGCAGGTGGTGGC
GGGCCATCCGCTCGCCCGGGACCTCGTGACGCGGGAAGGCGGCAC
TGTGCGCAGCCGCGTGGTCGGCCGGCTGCTGGAAACCGCGCGCAC
CCTGATCGCCATGGAGGGCTGGGTGAAGGAACTGCGGCCCGAAGG
GCCCTGGTGCGCCCAGGGCCACCTGCCCCAGGAAGGCCGCGCCTT
CGGCCTCACCGAGGCGGCGCGCGGGGCGCTCGGCCACTGGATGGT
GGTGGAGAAGGGCCGCATTGCCCGCTACCAGATCATCGCCCCCAC
CACCTGGAACTTCTCCCCCCGCGACGGCGCGGGCCTGCCCGGCCC
GCTGGAGACGGCCCTGGTGGGCGCGCCCGTGCGGCAGGGAGAGAC
GACGCCCGTGAGCGTGCAGCACATCGTGCGCTCCTTCGACCCGTG
CATGGTCTGCACTGTGCATTGA
SEQ ID NO: 15:
Amino acid sequence of Periplasmic [NiFeSe]
hydrogenase large subunit:
MSAETRRLVVGPFNRVEGDLEVRLDVQDGRVQQAFVSSPLFRGFE
RILEGRDPRDALVIAPRICGICSVSQSHAAALALAGLQGIAPTHD
GRIATNLIVAAENVADHLTHFHVFFMPDFARAVYEDRPWFAQAAR
RFKANQGVSVRRALQTRATLLHVLGTLAGRWPHTLALQPGGVTRS
ADQHDRMRLLATLKAVRAALEETLFGAPLEEVAALDGAAAVEAWR
ANGPEGDFRLFLEIAADLELDRLGRAHDRFLSFGAYAQDEGRLYG
AGTFEAGTAGGLDPNAITEDHAFARMEDRAAPHAPFDGSTFPDAD
DTEGYTWCKAPRLAGLPFETGAFARQVVAGHPLARDLVTREGGTV
RSRVVGRLLETARTLIAMEGWVKELRPEGPWCAQGHLPQEGRAFG
LTEAARGALGHWMVVEKGRIARYQIIAPTTWNFSPRDGAGLPGPL
ETALVGAPVRQGETTPVSVQHIVRSFDPCMVCTVH
SEQ ID NO: 16:
Nucleotide sequence of Periplasmic [NiFeSe]
hydrogenase small subunit
ACGGGGGAGGAAGCCCGCGCCATCTTCGACGCCATCCTTGCCGGC
GTTATCGTCCTCGACGCCCTGTGCGTGGAAGGCGCGCTGCTGCGC
GGGCCGAACGGCACCGGGCGCTTCCATGTGCTGGCGGGCACGGAC
ACCCCCACCATCGACTGGGCGCGGCAGCTCGCCGGCATGGCGCGC
CACGTGGTGGCGGTGGGCACCTGCGCCGCCTATGGGGGCGTGACG
GCGGCGGGCATCAACCCCACCGATGCCTGCGGCCTCCAGTTCGAC
GGACGCCGGAAGGGTGGGGCGCTGGGGGGGGACTTCCGCTCCCGC
TCGGGGCTTCCGGTCATCAATGTGGCCGGCTGCCCCACCCATCCC
AACTGGGTGACGGAAACCCTGATGCTGCTCGCCTGCGGCCTGCTG
GGCGAGGCCGACCTCGACGTCTATGGCCGCCCGCGCTTCTATGCG
GACCTGCTGGTGCATCACGGCTGCCCGCGCAACGAATACTATGAA
TACAAGGCGAGCGCCGAGAAGATGAGCGACCTCGGCTGCATGATG
GAGCATCTGGGCTGCCTCGGCACCCAGGCCCACGCCGACTGCAAC
ACGCGCCTTTGGAATGGCGAGGGCTCGTGCACCCGCGGCGGCTAT
GCCTGCATCAACTGCACGGCGCCGGAATTCGAGGAGCCGGGCCAC
GCCTTCCTGGAGACGCCCAAGATCGGCGGCATCCCCATCGGCCTG
CCCACCGACATGCCCAAGGCCTGGTTCATCGCCTTGTCCTCCCTC
GCCAAGGCGGCGACGCCGGAGCGGCTGCGCAAGAACGCGGTGTCC
GACCATGTGGTCACGCCGCCCGCCGTCAAGGACATCAAGCGGCGA
TGA
SEQ ID NO: 17:
Amino acid sequence of Periplasmic [NiFeSe]
hydrogenase small subunit
MSTPFSVLWLQSGGCGGCTMSLLCAEAPDLATTLDAAGIGFLWHP
ALSEETGEEARAIFDAILAGVIVLDALCVEGALLRGPNGTGRFHV
LAGTDTPTIDWARQLAGMARHVVAVGTCAAYGGVTAAGINPTDAC
GLQFDGRRKGGALGADFRSRSGLPVINVAGCPTHPNWVTETLMLL
ACGLLGEADLDVYGRPRFYADLLVHHGCPRNEYYEYKASAEKMSD
LGCMMEHLGCLGTQAHADCNTRLWNGEGSCTRGGYACINCTAPEF
EEPGHAFLETPKIGGIPIGLPTDMPKAWFIALSSLAKAATPERLR
KNAVSDHVVTPPAVKDIKRR

In another embodiment, the bacterial strain used in the process of the invention comprises a gene encoding an ATP synthase gamma chain atpG_1 having the sequence set forth in SEQ ID NO: 19 or a sequence having more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:19.

In another embodiment, the bacterial strain used in the process of the invention comprises a gene encoding an ATP synthase subunit alpha atpA_1 having the sequence set forth in SEQ ID NO:21 or a sequence having more than 78% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:21.

In another embodiment, the bacterial strain used in the process of the invention comprises a gene encoding an ATP synthase subunit b atpF_1 having the sequence set forth in SEQ ID NO:23 or a sequence having more than 62% identity, e.g. more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:23.

In another embodiment, the bacterial strain used in the process of the invention comprises a gene encoding an ATP synthase subunit c, sodium ion specific atpE_1 having the sequence set forth in SEQ ID NO:25 or a sequence having more than 90% identity, such as more than 95% identity, e.g. more than 96% identity, such as more than 97% identity, e.g. more than 98%, such as more than 99% sequence identity to the sequence set forth in SEQ ID NO:25.

In another embodiment, the bacterial strain used in the process of the invention comprises a gene encoding an ATP synthase subunit a atpB_1 having the sequence set forth in SEQ ID NO:27 or a sequence having more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:27.

In another embodiment, the bacterial strain used in the process of the invention comprises a gene encoding an ATP synthase epsilon chain atpC_1 having the sequence set forth in SEQ ID NO:29 or a sequence having more than 71% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:29.

In another embodiment, the bacterial strain used in the process of the invention comprises a gene encoding an ATP synthase subunit beta atpD_1 having the sequence set forth in SEQ ID NO:31 or a sequence having more than 84% identity, e.g. more than 90% identity, such as more than 95% identity, e.g. more than 96% identity, such as more than 97% identity, e.g. more than 98%, such as more than 99% sequence identity to the sequence set forth in SEQ ID NO:31.

In another embodiment, the bacterial strain used in the process of the invention comprises a gene encoding an ATP synthase subunit beta atpD_2 having the sequence set forth in SEQ ID NO:33 or a sequence having more than 97% identity, e.g. more than 98%, such as more than 99% sequence identity to the sequence set forth in SEQ ID NO:33.

In another embodiment, the bacterial strain used in the process of the invention comprises a gene encoding an ATP synthase gamma chain atpG_2 having the sequence set forth in SEQ ID NO:35 or a sequence having more than 86% identity, e.g. more than 90% identity, such as more than 95% identity, e.g. more than 96% identity, such as more than 97% identity, e.g. more than 98%, such as more than 99% sequence identity to the sequence set forth in SEQ ID NO:35.

In another embodiment, the bacterial strain used in the process of the invention comprises a gene encoding an ATP synthase subunit alpha atpA_2 having the sequence set forth in SEQ ID NO:37 or a sequence having more than 98%, such as more than 99% sequence identity to the sequence set forth in SEQ ID NO:37.

In another embodiment, the bacterial strain used in the process of the invention comprises a gene encoding an ATP synthase subunit delta atpH having the sequence set forth in SEQ ID NO:39 or a sequence having more than 85% identity, e.g. more than 90% identity, such as more than 95% identity, e.g. more than 96% identity, such as more than 97% identity, e.g. more than 98%, such as more than 99% sequence identity to the sequence set forth in SEQ ID NO:39.

In another embodiment, the bacterial strain used in the process of the invention comprises a gene encoding an ATP synthase subunit b atpF_2 having the sequence set forth in SEQ ID NO:41 or a sequence having more than 87% identity, e.g. more than 90% identity, such as more than 95% identity, e.g. more than 96% identity, such as more than 97% identity, e.g. more than 98%, such as more than 99% sequence identity to the sequence set forth in SEQ ID NO:41.

In another embodiment, the bacterial strain used in the process of the invention comprises a gene encoding an ATP synthase subunit b′ atpG_3 having the sequence set forth in SEQ ID NO:43 or a sequence having more than 81% identity, e.g. more than 90% identity, such as more than 95% identity, e.g. more than 96% identity, such as more than 97% identity, e.g. more than 98%, such as more than 99% sequence identity to the sequence set forth in SEQ ID NO:43.

In another embodiment, the bacterial strain used in the process of the invention comprises a gene encoding ATP synthase subunit c atpE_2 having the sequence set forth in SEQ ID NO:45 or a sequence having more than 98%, such as more than 99% sequence identity to the sequence set forth in SEQ ID NO:45.

In another embodiment, the bacterial strain used in the process of the invention comprises a gene encoding an ATP synthase subunit a atpB_2 having the sequence set forth in SEQ ID NO:47 or a sequence having more than 92% identity, such as more than 95% identity, e.g. more than 96% identity, such as more than 97% identity, e.g. more than 98%, such as more than 99% sequence identity to the sequence set forth in SEQ ID NO:47.

In another embodiment, the bacterial strain used in the process of the invention comprises a gene encoding an ATP synthase protein I atpI having the sequence set forth in SEQ ID NO:49 or a sequence having more than 60% identity, e.g. more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:49.

SEQ ID NO: 18:
Nucleotide sequence of ATP synthase gamma
chain atpG_1
GTGACCGAGCGCCTGTCCGACGTCAACGCCCGCATCGCCTCGGTG
CGGCAGCTCTCATCGGTCATCACGGCCATGCGGGGCATTGCGGCG
GCGCGGGCGCGGGAGGCGCGGGGTCGGCTCGACGGCATCCGCGCC
TATGCGCAGACCATCGCCGAGGCCATCGGCCATGTGCTCGCCGTG
CTGCCCGAGGAGGCCCGCGCCCGGTCCTCCGGGCACCGGCATCGG
GGCCATGCGGTCATCGCCCTGTGCGCGGAGCAGGGCTTTGCCGGC
GTCTTCAACGAGCGGGTGCTGGACGAGGCCGCCCGGCTGCTGACC
GGCGGGGCGGGGCCGGCCGAGCTGCTGCTGGTGGGCGACCGGGGC
CTGATGGTGGCCCGCGAGCGGGGGCTCGATGTCTCCTGGTCGGTG
CCCATGGTGGCCCATGCGGGCCAGGCCTCGGCGCTGGCGGACCGC
ATCAGCGAGGAGCTCTACCGGCGGATCGATGCGGGACGGGTGACG
CGGGTGTCGGTGGTGCACGCCGAGCCCGCCGCGTCCGCCGCCATC
GAGACGGTGGTGAAAGTGCTGGTGCCGTTCGACTTCGCCCGCTTC
CCCCTGGCGCGGGTGGCATCCGCCCCGCTCATGACCATGCCGCCG
CCGCGGCTGCTGGCCCAGCTGTCGGAGGAATATGTGTTCGCCGAG
CTGTGCGAGGCGCTCACCTTGTCCTTCGCGGCGGAGAACGAGGCC
CGCATGCGGGCCATGATCGCCGCCCGCGCCAATGTGGCCGATACC
CTGGAGGGCCTCGTCGGCCGCGCCCGGCAGATGCGCCAGGAGGAG
ATCACCAACGAGATCATCGAGCTGGAAGGCGGCGCCGGCAGCGCC
CGGCATGCGGATTGA
SEQ ID NO: 19:
Amino acid sequence of ATP synthase gamma
chain atpG_1
MTERLSDVNARIASVRQLSSVITAMRGIAAARAREARGRLDGIRA
YAQTIAEAIGHVLAVLPEEARARSSGHRHRGHAVIALCAEQGFAG
VFNERVLDEAARLLTGGAGPAELLLVGDRGLMVARERGLDVSWSV
PMVAHAGQASALADRISEELYRRIDAGRVTRVSVVHAEPAASAAI
ETVVKVLVPFDFARFPLARVASAPLMTMPPPRLLAQLSEEYVFAE
LCEALTLSFAAENEARMRAMIAARANVADTLEGLVGRARQMRQEE
ITNEIIELEGGAGSARHAD
SEQ ID NO: 20:
Nucleotide sequence of ATP synthase subunit
alpha atpA_1
ATGAGCACGGGCGCGCAAGCGAGCGAGGATTGGCTCACCCGGAGC
CGGGCGGCCCTGGCCGGGACGCGCCTTTCCCAGCAATCCCAATCG
GTGGGCCGGGTGGAGGAGATGGCCGACGGCATCGCCCGCGTCTCC
GGCCTGCCGGATGTGCGGCTCGACGAGCTTCTCACCTTCGAGGGC
GGCCAGACCGGCTATGCCCTCACCCTCGATCGCACCGAGATCGCC
GTGGTGCTGCTGGATGACGCCTCCGGCGTGGAGGCGGGCGCCCGG
GTGTTCGGCACCGGCGAGGTGGTGAAGGTGCCGGTGGGGCCGGGG
CTGCTGGGCCGCATCGTCGACCCCCTCGGCCGGCCCATGGACCGC
TCCGAGCCGGTGGTGGCGCAGGCGCACCATCCCATCGAGCGGCCG
GCGCCGGCCATCATCGCCCGCGACCTGGTCTCGCAGCCGGTTCAG
ACCGGCACGCTGGTGGTGGATGCGCTGTTCTCCCTCGGCCGGGGC
CAGCGCGAGCTCATCATCGGCGACCGGGCTACCGGCAAGACCGCC
ATCGCGGTGGACACCATCATCAGCCAGAAGCATTCGGACATCGTG
TGCATCTACGTGGCGGTGGGCCAGCGCGCCGCCGCCGTGGAGCGG
GTGGTGGAGGCGGTGCGCGCCCACGGGGCGATCGAGCGCTGCATC
TTCGTGGTCGCCTCGGCCGCCGCCTCGCCAGGGCTGCAATGGATC
GCGCCGTTCGCCGGCATGACCATGGCGGAATATTTCCGCGACAAC
GGCCAGCATGCGCTCATCATCATCGATGATCTCACCAAGCATGCG
GCCACCCATCGCGAGCTGGCGCTGCTCACCCACGAGCCGCCGGGC
CGCGAGGCCTATCCCGGCGACATCTTCTATGTGCACGCCCGCCTT
CTGGAGCGGGCCGCCAAGCTCTCCGCCGAGCTGGGCGGTGGCTCG
CTCACGGCCCTGCCCATCGCGGAGACGGACGCGGGAAACCTCTCC
GCCTATATCCCCACCAACCTCATCTCCATCACCGATGGGCAGATC
GTGCTGGATTCGCGGCTGTTCGCGGCCAACCAGCGCCCGGCGGTG
GATGTGGGCCTCTCCGTGAGCCGGGTGGGCGGCAAGGCGCAGCAT
CCCGCGCTTCGGGCCGTGTCCGGGCGCATCCGGCTCGATTATTCC
CAGTTCCTGGAGCTGGAAATGTTCACCCGCTTCGGCGGCATCACC
GATACCCGCGTGAAGGCGCAGATCACCCGGGGCGAGCGCATCCGC
GCGCTGCTCACCCAGCCGCGCTTTTCCACCCTGCGCCTTCAGGAC
GAGGTGGCGCTGCTGGCCGCGCTGGCGGAGGGGGTGTTCGACACT
TTGGCCCCGGGGCTGATGGGCGCCGTGCGTGCCCGCATTCCGGCC
CAGCTGGATGCGCAGGTGAAGGACGTGGCCTCGGCCCTCGCCGAG
GGCAAGGTGCTGGAGGAGGGCTTGCACGCCCGTCTCGTGGCGGCC
GTGCGGGCCGTCGCGGCGGACGTGGCCGCGACCGCGAAGGCCGGG
CCGTGA
SEQ ID NO: 21:
Amino acid sequence of ATP synthase subunit
alpha atpA_1
MSTGAQASEDWLTRSRAALAGTRLSQQSQSVGRVEEMADGIARVS
GLPDVRLDELLTFEGGQTGYALTLDRTEIAVVLLDDASGVEAGAR
VFGTGEVVKVPVGPGLLGRIVDPLGRPMDRSEPVVAQAHHPIERP
APAIIARDLVSQPVQTGTLVVDALFSLGRGQRELIIGDRATGKTA
IAVDTIISQKHSDIVCIYVAVGQRAAAVERVVEAVRAHGAIERCI
FVVASAAASPGLQWIAPFAGMTMAEYFRDNGQHALIIIDDLTKHA
ATHRELALLTHEPPGREAYPGDIFYVHARLLERAAKLSAELGGGS
LTALPIAETDAGNLSAYIPTNLISITDGQIVLDSRLFAANQRPAV
DVGLSVSRVGGKAQHPALRAVSGRIRLDYSQFLELEMFTRFGGIT
DTRVKAQITRGERIRALLTQPRFSTLRLQDEVALLAALAEGVFDT
LAPGLMGAVRARIPAQLDAQVKDVASALAEGKVLEEGLHARLVAA
VRAVAADVAATAKAGP
SEQ ID NO: 22:
Nucleotide sequence of ATP synthase subunit
b atpF_1
ATGCAGATCGACTGGTGGACGCTGGGCCTGCAGACGGTCAACGTC
CTCGTTCTCATCTGGCTCCTGAGCCGCTTCCTGTTCAAGCCGGTG
GCGCAGGTCATCGCGCAGCGCCGTGCCGAGATCGAGAAGCTGGTG
GAGGATGCGCGCGCCGCCAAGGCCGCCGCCGAGGCCGAGCGGGAC
ACGGCGAAGGCGGAGGAGGCGCGCCTTGCCGCCGAGCGCGGCGCC
CGCATGGCGGCGGTCGCCAAGGAGGCGGAGGCGCAGAAGGCGGCA
TTGCTGGCCGCCGCCAAGACCGAGGCCGAGGCCCTGCACGCGGCC
GCGGAAGCGGCCATCGTCCGGGCGCGGGCGAGCGAGGAGGAAGCC
GCCGCCGACCGCGCCAGCCGCCTTGCCGTGGACATCGCCGCCAAG
CTGCTGGACCGGCTGCCCGACGACGCCCGGGTCGCGGGCTTCATC
GATGGCCTCGCCGAGGGGCTTGAAGCCCTGCCCGAGGCGAGCCGG
GCGGTGATCGGCGTCGACGGCGCGCCAGTGCGCGTGACGGCCGCG
CGCGCCCTTATGCCGGCGGAGGAGGAGGCCTGCCGCACGCGGCTC
TCCCAGGCGCTGGGCCGTCCGGTGACGCTGGCCGTGACCATCGAC
CCCGCCCTCATCGCCGGCCTGGAGATGGAGACGCCCCACGCGGTG
GTGCGCAATTCCTTCAAGGCCGATCTCGACCGCGTCACCGCGGCG
CTCACCCATCATGGGACCTGA
SEQ ID NO: 23:
Amino acid sequence of ATP synthase subunit
b atpF_1
MQIDWWTLGLQTVNVLVLIWLLSRFLFKPVAQVIAQRRAEIEKLV
EDARAAKAAAEAERDTAKAEEARLAAERGARMAAVAKEAEAQKAA
LLAAAKTEAEALHAAAEAAIVRARASEEEAAADRASRLAVDIAAK
LLDRLPDDARVAGFIDGLAEGLEALPEASRAVIGVDGAPVRVTAA
RALMPAEEEACRTRLSQALGRPVTLAVTIDPALIAGLEMETPHAV
VRNSFKADLDRVTAALTHHGT
SEQ ID NO: 24:
Nucleotide sequence of ATP synthase subunit c,
sodium ion specific atpE_1
ATGACTGTCGAGATGGTCAGCATCTTCGCGGCGGCGCTCGCCGTC
TCCTTCGGCGCCATCGGGCCGGCCCTGGGCGAGGGCCGGGCGGTG
GCCGCGGCCATGGACGCCATCGCCCGCCAGCCGGAGGCGGCCGGA
ACCTTGTCGCGCACGCTCTTCGTCGGCCTCGCCATGATCGAGACC
ATGGCGATCTACTGCCTGGTGATCGCGCTCCTGGTGCTCTTCGCC
AATCCGTTCGTGAAGTGA
SEQ ID NO: 25:
Amino acid sequence of ATP synthase subunit c,
sodium ion specific atpE_1
MTVEMVSIFAAALAVSFGAIGPALGEGRAVAAAMDAIARQPEAAG
TLSRTLFVGLAMIETMAIYCLVIALLVLFANPFVK
SEQ ID NO: 26:
Nucleotide sequence of ATP synthase subunit a
atpB_1
ATGGGCTCGCCGCTGATCCTCGAACCCCTGTTCCATATCGGGCCC
GTGCCCATCACCGCGCCGGTGGTGGTCACCTGGCTCATCATGGCC
GCCTTCATTGGGCTGGCGCGGCTCATCACCCGGAAGCTTTCCACC
GATCCCACCCGGACCCAGGCGGCGGTGGAAACGGTGCTGACCGCC
ATCGATTCCCAGATCGCCGACACCATGCAGGCCGATCCCGCGCCT
TATCGCGCGCTCATCGGCACCATCTTCCTTTATGTGCTGGTGGCC
AACTGGTCCTCGCTCATCCCGGGCATCGAGCCGCCCACGGCGCAT
ATCGAGACCGATGCGGCGCTCGCTTTCATCGTGTTCGCCGCCACC
ATCGGGTTCGGGTTGAAGACAAGGGGTGTGAAGGGCTATCTCGCC
ACCTTCGCCGAACCCTCCTGGGTGATGATCCCGCTCAATGTGGTG
GAGCAGATCACCCGGACCTTCTCGCTCATCGTGCGCCTGTTCGGC
AACATCATGAGCGGGGTGTTCGTGGTCGGCATCATCCTGTCCCTC
GCCGGGCTGCTGGTGCCCATCCCCCTCATGGCGCTCGATCTCCTG
ACCGGCGCCGTGCAGGCCTACATCTTCGCGGTGCTGGCCTGCGTG
TTCATCGGCGCGGCCATTGGCGAGGCGCCGGCAAAGCCCCAATCG
AAGGAGCCAGGGAAAACATCATGA
SEQ ID NO: 27:
Amino acid sequence of ATP synthase subunit a
atpB_1
MGSPLILEPLFHIGPVPITAPVVVTWLIMAAFIGLARLITRKLST
DPTRTQAAVETVLTAIDSQIADTMQADPAPYRALIGTIFLYVLVA
NWSSLIPGIEPPTAHIETDAALAFIVFAATIGFGLKTRGVKGYLA
TFAEPSWVMIPLNVVEQITRTFSLIVRLFGNIMSGVFVVGIILSL
AGLLVPIPLMALDLLTGAVQAYIFAVLACVFIGAAIGEAPAKPQS
KEPGKTS
SEQ ID NO: 28:
Nucleotide sequence of ATP synthase epsilon
chain atpC_1
GTGAGCGCGCCGCTGCACCTCACCATCACCACGCCGGCCGCCGTT
CTGGTGGACCGTGCCGACATCGTGGCCCTGCGTGCCGAGGACGAG
AGCGGCAGCTTCGGCATCCTGCCCGGCCATGCGGATTTCCTGACC
GTTCTGGAGGCCTGCGTGGTGCGCTTCAAGGATGGGGCCGACGGC
GTGCATTATTGTGCTCTCAGTGGTGGCGTGCTGTCGGTCGAGGAG
GGCCGGCGCATCGCCATCGCCTGCCGTCAGGGCACGGTGAGCGAC
GACCTGGTCGCCCTGGAAGGGGCGGTGGACGCCATGCGTTCGGCG
GAGAGCGATGCCGACAAGCGGGCCCGGGTGGAGCAGATGCGCCTT
CATGCCCACGCCGTGCGCCAGCTCCTGCACTATCTGCGGCCCGGC
CGGGCCGGCGGCGTGGCGCCGGCCGCCGCGCCGGAGGAGGGGCCG
TCATGA
SEQ ID NO: 29:
Amino acid sequence of ATP synthase epsilon
chain atpC_1
MSAPLHLTITTPAAVLVDRADIVALRAEDESGSFGILPGHADFLT
VLEACVVRFKDGADGVHYCALSGGVLSVEEGRRIAIACRQGTVSD
DLVALEGAVDAMRSAESDADKRARVEQMRLHAHAVRQLLHYLRPG
RAGGVAPAAAPEEGPS
SEQ ID NO: 30:
Nucleotide sequence of ATP synthase subunit
beta atpD_1
ATGGCAGCGGCAGATGAGGAGGCGCAATCGGCCGCCGGCCCCGCC
TCGGGCCGGGTGGTGGCCGTGCGCGGCGCGGTGATCGACATCGCC
TTTGCCCAGCCTCCGCTGCCGCCGCTGGACGACGCCCTTCTCATC
ACCGACGGCCGGGGCGGCACGGTGCTGGTGGAGGTGCAGAGCCAT
ATGGATCGGCACACGGTGCGCGCCATCGCCCTTCAGGCCACCACC
GGCCTCAGCCGGGGGCTGGAGGCGGCGCGGGTGGGCGGGCCGGTG
AAGGTGCCGGTGGGAGACCATGTGCTCGGCCGCCTCCTGGATGTC
ACCGGCGCCATCGGCGACAAGGGGGGCCGCTGCCGGCCGACGTGC
CCACGCGGCCGATCCACCACGCGCCGCCATCCTTCGCCGCGCAGG
GCGGCACGTCCGATCTGTTTCGCACCGGCATCAAGGTCATCGACC
TCCTGGCGCCCCTCGCCCAGGGCGGCAAGGCGGCCATGTTCGGCG
GGGCCGGCGTGGGCAAGACCGTGCTGGTGATGGAGCTGATCCACG
CCATGGTGGCGAGCTACAAGGGCATCTCGGTGTTTGCCGGCGTGG
GGGAGCGCTCCCGCGAGGGCCACGAGATGCTGCTGGACATGACCG
ATTCCGGCGTGCTCGACCGCACCGTTCTGGTCTATGGCCAGATGA
ACGAGCCCCCCGGGGCCCGCTGGCGGGTGCCCATGACGGCGCTGA
CCATCGCCGAATATTTCCGCGACGAGAAGCACCAGAACGTCCTGC
TGCTGATGGACAACATCTTCCGCTTCGTCCAGGCGGGGGCGGAGG
TCTCCGGCCTTTTGGGCCGTCCGCCCTCCCGGGTGGGATACCAGC
CGACGCTGGCGAGCGAGGTGGCGGCGCTCCAGGAACGCATCACCT
CCGTGGGCGAGGCCTCGGTGACCGCCATCGAGGCGGTCTACGTGC
CGGCGGATGACTTCACCGATCCCGCCGTGACCACCATCGCCGCCC
ACGTGGATTCCATGGTGGTGCTCTCCCGCGCCATGGCGGCGGAGG
GCATGTATCCGGCGGTGGACCCCATCTCCTCCTCGTCGGTGCTGC
TCGACCCGCTCATCGTGGGGGACGAGCATGCGCGCGTCGCCAACG
AGGTGCGCCGGACCATCGAGCATTATCGCGAGCTTCAGGATGTGA
TCTCGCTGCTGGGCATGGAGGAATTGGGCACCGAGGATCGCCGCA
TCGTGGAGCGGGCGCGCCGGCTCCAGCGCTTCCTCACCCAGCCCT
TCACGGTCACCGAGGCCTTCACCGGCGTGCCCGGCCGCTCGGTGG
CCATCGCCGACACCATCGCCGGCTGCAGGATGATCCTGTCCGGCG
CCTGCGACGACTGGCAGGAAAGCGCCCTCTACATGGTGGGCACCA
TCGACGAGGCCCGCCAGAAGGAGGAGGCCGCTCGCGCCAAGGCGG
GGCAGGGCGCCCCGGCCGGGACGGCAGCCGAGACGGCGGAGGCCG
CCCCGTGA
SEQ ID NO: 31:
Amino acid sequence of ATP synthase subunit
beta atpD_1
MAAADEEAQSAAGPASGRVVAVRGAVIDIAFAQPPLPPLDDALLI
TDGRGGTVLVEVQSHMDRHTVRAIALQATTGLSRGLEAARVGGPV
KVPVGDHVLGRLLDVTGAIGDKGGPLPADVPTRPIHHAPPSFAAQ
GGTSDLFRTGIKVIDLLAPLAQGGKAAMFGGAGVGKTVLVMELIH
AMVASYKGISVFAGVGERSREGHEMLLDMTDSGVLDRTVLVYGQM
NEPPGARWRVPMTALTIAEYFRDEKHQNVLLLMDNIFRFVQAGAE
VSGLLGRPPSRVGYQPTLASEVAALQERITSVGEASVTAIEAVYV
PADDFTDPAVTTIAAHVDSMVVLSRAMAAEGMYPAVDPISSSSVL
LDPLIVGDEHARVANEVRRTIEHYRELQDVISLLGMEELGTEDRR
IVERARRLQRFLTQPFTVTEAFTGVPGRSVAIADTIAGCRMILSG
ACDDWQESALYMVGTIDEARQKEEAARAKAGQGAPAGTAAETAEA
AP
SEQ ID NO: 32:
Nucleotide sequence of ATP synthase subunit
beta atpD_2
ATGGCGAACAAGGTCGGACGCATCACCCAGATCATCGGCGCCGTC
GTCGACGTGCAGTTCGACGGGCATCTGCCGGCGATTCTCAACGCG
ATCGAGACCACCAACCAGGGCAACCGGCTGGTGCTCGAAGTGGCT
CAGCATCTCGGCGAGAACACCGTGCGCTGCATCGCCATGGATGCC
ACTGAAGGCCTGGTGCGTGGCCAGGAGGTGGCCGACACCGATGCG
CCCATCCAGGTGCCCGTGGGCGCCGCCACCCTCGGCCGCATCATG
AACGTGATCGGCGAGCCGGTGGACGAGCTGGGCCCCATCGAGGGC
GAAGCGCTGCGCGGCATCCATCAGCCGGCCCCCTCCTATGCGGAG
CAGGCCACGGAAGCTGAGATCCTCGTCACCGGCATCAAGGTGGTG
GATCTGCTGGCGCCCTATTCCAAGGGCGGCAAGGTGGGCCTGTTC
GGCGGCGCCGGCGTGGGCAAGACCGTGCTCATCATGGAGCTGATC
AACAACGTGGCCAAGGCGCACGGCGGCTATTCCGTGTTCGCCGGC
GTGGGTGAGCGCACCCGCGAGGGCAACGACCTCTACCACGAGATG
ATCGAGTCCAACGTGAACAAGGACCCGCACGAGAACAATGGCTCG
GCGGCCGGTTCCAAGTGCGCCCTGGTCTATGGCCAGATGAACGAG
CCGCCCGGCGCCCGCGCCCGCGTGGCCCTCACCGGCCTCACCGTC
GCCGAGCATTTCCGCGACCAGGGCCAGGACGTGCTGTTCTTCGTG
GACAACATCTTCCGCTTCACCCAGGCGGGCTCCGAGGTGTCGGCG
CTTCTCGGCCGCATCCCCTCGGCGGTGGGCTACCAGCCGACGCTG
GCCACCGACATGGGCCAGCTGCAGGAGCGCATCACCACCACCACC
AAGGGCTCCATCACCTCGGTGCAGGCCATCTACGTGCCGGCGGAC
GATCTGACCGATCCGGCGCCGGCCGCCTCCTTCGCCCATCTGGAC
GCCACCACGGTGCTGTCGCGCTCCATCGCGGAGAAGGGCATCTAC
CCGGCGGTGGATCCGCTGGACTCCACCTCGCGCATGCTGTCTCCC
GCCATCCTCGGCGACGAGCACTACAACACCGCGCGCCAGGTGCAG
CAGACCCTGCAGCGCTACAAGGCGCTCCAGGACATCATCGCCATC
CTGGGCATGGACGAACTCTCCGAAGAGGACAAGCTCACCGTGGCC
CGCGCCCGCAAGATCGAGCGCTTCCTCTCCCAGCCCTTCCACGTG
GCCGAGGTGTTCACCGGTTCGCCCGGCAAGCTGGTCGACCTCGCC
GACACCATCAAGGGCTTCAAGGGCCTGGTGGACGGCAAGTACGAC
TACCTGCCCGAGCAGGCCTTCTACATGGTGGGCACCATCGAAGAA
GCCATCGAGAAGGGCAAGAAGCTGGCGGCCGAGGCGGCCTGA
SEQ ID NO: 33:
Amino acid sequence of ATP synthase subunit
beta atpD_2
MANKVGRITQIIGAVVDVQFDGHLPAILNAIETTNQGNRLVLEVA
QHLGENTVRCIAMDATEGLVRGQEVADTDAPIQVPVGAATLGRIM
NVIGEPVDELGPIEGEALRGIHQPAPSYAEQATEAEILVTGIKVV
DLLAPYSKGGKVGLFGGAGVGKTVLIMELINNVAKAHGGYSVFAG
VGERTREGNDLYHEMIESNVNKDPHENNGSAAGSKCALVYGQMNE
PPGARARVALTGLTVAEHFRDQGQDVLFFVDNIFRFTQAGSEVSA
LLGRIPSAVGYQPTLATDMGQLQERITTTTKGSITSVQAIYVPAD
DLTDPAPAASFAHLDATTVLSRSIAEKGIYPAVDPLDSTSRMLSP
AILGDEHYNTARQVQQTLQRYKALQDIIAILGMDELSEEDKLTVA
RARKIERFLSQPFHVAEVFTGSPGKLVDLADTIKGFKGLVDGKYD
YLPEQAFYMVGTIEEAIEKGKKLAAEAA
SEQ ID NO: 34:
Nucleotide sequence of ATP synthase gamma
chain atpG_2
ATGGCGAGTCTGAAGGACCTGAGAAACCGCATTGCCTCGGTGAAG
GCGACGCAGAAGATCACCAAGGCGATGCAGATGGTCGCCGCGGCG
AAGCTGCGTCGCGCCCAGGCGGCGGCTGAAGCGGCCCGTCCCTAT
GCGGAACGCATGGAGACGGTGCTCGGAAATCTTGCCTCCGGCATG
GTGGTGGGCGCGCAGGCGCCTGTTCTCATGACCGGGACGGGCAAG
AGCGACACCCACCTGCTGCTGGTGTGCACCGGCGAGCGCGGCCTG
TGCGGCGCCTTCAACTCGTCCATCGTGCGCTTCGCCCGCGAGCGG
GCGCAGCTGCTGCTGGCCGAGGGCAAGAAGGTGAAAATCCTGTGC
GTGGGCCGCAAGGGCCACGAGCAGCTGCGCCGCATCTACCCGGAC
AACATCATCGACGTGGTGGACCTGCGCGCGGTGCGCAACATCGGC
TTCAAGGAGGCCGACGCCATCGCCCGCAAGGTGCTGGCCCTGCTC
GATGAAGGCGCATTCGACGTCTGCACGCTCTTCTACTCCCACTTC
AGGAGCGTGATCGCCCAGGTGCCGACGGCCCAGCAGCTCATTCCG
GCCACCTTCGACGAGCGGCCGGCCGTCGCCGATGCGCCGGTCTAT
GAATATGAGCCGGAGGAGGAGGAGATCCTCGCCGAGCTGCTGCCG
CGCAACGTGGCGGTGCAGATCTTCAAGGCCCTCCTCGAGAACCAG
GCTTCTTTCTATGGCTCCCAGATGAGCGCCATGGACAACGCCACG
CGCAATGCGGGCGAGATGATCAAGAAGCAGACGCTCACCTACAAC
CGTACCCGCCAGGCCATGATCACGAAGGAACTCATCGAGATCATC
TCCGGCGCCGAGGCCGTCTGA
SEQ ID NO: 35:
Amino acid sequence of ATP synthase gamma
chain atpG_2
MASLKDLRNRIASVKATQKITKAMQMVAAAKLRRAQAAAEAARPY
AERMETVLGNLASGMVVGAQAPVLMTGTGKSDTHLLLVCTGERGL
CGAFNSSIVRFARERAQLLLAEGKKVKILCVGRKGHEQLRRIYPD
NIIDVVDLRAVRNIGFKEADAIARKVLALLDEGAFDVCTLFYSHF
RSVIAQVPTAQQLIPATFDERPAVADAPVYEYEPEEEEILAELLP
RNVAVQIFKALLENQASFYGSQMSAMDNATRNAGEMIKKQTLTYN
RTRQAMITKELIEIISGAEAV
SEQ ID NO: 36:
Nucleotide sequence of ATP synthase subunit
alpha atpA_2
ATGGACATTCGAGCCGCTGAAATCTCTGCCATCCTGAAAGAGCAG
ATCCAGAATTTCGGCCAGGAGGCGGAAGTCTCCGAGGTGGGTCAG
GTTCTGTCCGTGGGTGACGGCATCGCGCGCGTCTACGGCCTCGAC
AACGTCCAGGCGGGCGAGATGGTCGAGTTCGAGAACGGCACGCGC
GGCATGGCGCTGAACCTCGAGCTCGACAATGTCGGCATCGTGATC
TTCGGTTCCGACCGCGAGATCAAGGAAGGCCAGACCGTCAAGCGG
ACCGGCGCCATCGTGGACGCCCCCGTCGGCAAGGGCCTGCTCGGC
CGCGTCGTGGACGCTCTCGGCAACCCGATCGACGGCAAGGGCCCG
ATCATGTTCACCGAGCGTCGCCGGGTCGACGTGAAGGCGCCGGGC
ATCATCCCGCGCAAGTCGGTGCACGAGCCCATGCAGACCGGCCTG
AAGGCCATCGATGCGCTCATCCCCATCGGCCGCGGCCAGCGCGAG
CTCATCATCGGCGACCGCCAGACCGGCAAGACCGCCGTGGCGCTC
GACTCGATCCTGAACCAGAAGCCCATCAACCAGGGCGACGACGAG
AAGGCCAAGCTCTACTGCGTCTATGTCGCGGTGGGCCAGAAGCGT
TCCACTGTCGCGCAGTTCGTGAAGGTGCTCGAGGAGCACGGCGCG
CTGGAATATTCCATCGTCGTCGCCGCCACCGCCTCGGACGCGGCC
CCCATGCAGTTCCTGGCGCCGTTCACCGGCACCGCCATGGGCGAG
TATTTCCGCGACAACGGCATGCACGCCCTCATCATCCATGATGAC
CTGTCCAAGCAGGCCGTGGCCTACCGCCAGATGTCGCTGCTGCTG
CGCCGCCCGCCGGGCCGCGAGGCCTATCCCGGCGATGTGTTCTAC
CTGCACTCCCGCCTCTTGGAGCGCGCCGCCAAGCTCAATGACGAG
CACGGCGCCGGCTCGCTGACCGCCCTGCCGGTGATCGAGACCCAG
GCCAACGACGTGTCGGCCTACATCCCGACCAACGTGATCTCCATC
ACCGACGGTCAGATCTTCCTTGAATCCGATCTGTTCTACCAGGGC
ATCCGCCCGGCGGTGAACGTGGGCCTGTCGGTGTCGCGCGTGGGC
TCTTCGGCCCAGATCAAGGCGATGAAGCAGGTGGCCGGCAAGATC
AAGGGCGAGCTCGCCCAGTATCGCGAGCTGGCGGCCTTCGCCCAG
TTCGGTTCGGACCTGGACGCGGCCACCCAGAAGCTGCTGAACCGC
GGCGCCCGCCTCACCGAGCTGCTGAAGCAGAGCCAGTTCTCGCCC
CTCAAGGTGGAGGAGCAGGTGGCGGTGATCTATGCCGGCACCAAT
GGCTATCTCGATCCGCTGCCGGTCTCCAAGGTGCGCGAGTTCGAG
CAGGGTCTGCTCCTGTCGCTGCGCTCGCAGCATCCGGAGATCCTG
GACGCCATCCGCACGTCCAAGGAGCTTTCCAAGGACACCGCCGAG
AAGCTGACGAAGGCCATCGACGCCTTCGCCAAGAGCTTCTCCTGA
SEQ ID NO: 37:
Amino acid sequence of ATP synthase subunit
alpha atpA_2
MDIRAAEISAILKEQIQNFGQEAEVSEVGQVLSVGDGIARVYGLD
NVQAGEMVEFENGTRGMALNLELDNVGIVIFGSDREIKEGQTVKR
TGAIVDAPVGKGLLGRVVDALGNPIDGKGPIMFTERRRVDVKAPG
IIPRKSVHEPMQTGLKAIDALIPIGRGQRELIIGDRQTGKTAVAL
DSILNQKPINQGDDEKAKLYCVYVAVGQKRSTVAQFVKVLEEHGA
LEYSIVVAATASDAAPMQFLAPFTGTAMGEYFRDNGMHALIIHDD
LSKQAVAYRQMSLLLRRPPGREAYPGDVFYLHSRLLERAAKLNDE
HGAGSLTALPVIETQANDVSAYIPTNVISITDGQIFLESDLFYQG
IRPAVNVGLSVSRVGSSAQIKAMKQVAGKIKGELAQYRELAAFAQ
FGSDLDAATQKLLNRGARLTELLKQSQFSPLKVEEQVAVIYAGTN
GYLDPLPVSKVREFEQGLLLSLRSQHPEILDAIRTSKELSKDTAE
KLTKAIDAFAKSFS
SEQ ID NO: 38:
Nucleotide sequence of ATP synthase subunit
delta atpH
GTGGCGGAAACGATCGTGTCAGGCATGGCGGGACGCTATGCGACC
GCGCTGTTCGAGCTGGCGGACGAAGCCGGTGCCATCGATTCCGTC
CAGGCGGATCTTGATCGCCTGTCCGGCCTTCTGGCCGAGAGCGCG
GATCTGGCGCGGCTGGTCAAGAGCCCGGTCTTCACCGCCGAGCAG
CAGCTCGGCGCGATGGCGGCCATTCTCGATCAAGCAGGCATTTCC
GGCCTTGCGGGCAAATTCGTGAAGCTGGTGGCGCAGAACCGCCGC
CTGTTCGCACTGCCGCGCATGATTGCCGAATACGCCGTCCTGGTG
GCCCGGAAGAAGGGCGAGACCTCGGCGAGCGTGACCGTTGCCACC
CCCCTGAGCGATGAGCATCTGGCCACGCTCAAGGCGGCCCTGGCT
GAAAAGACCGGCAAGGACGTGAAGCTCGACGTCACCGTCGATCCG
TCCATCCTCGGTGGTCTCATCGTGAAGCTCGGCTCGCGCATGGTC
GATGCTTCCCTGAAGACCAAACTCAATTCTATCCGGCATGCGATG
AAAGAGGTCCGCTGA
SEQ ID NO: 39:
Amino acid sequence of ATP synthase subunit
delta atpH
MAETIVSGMAGRYATALFELADEAGAIDSVQADLDRLSGLLAESA
DLARLVKSPVFTAEQQLGAMAAILDQAGISGLAGKFVKLVAQNRR
LFALPRMIAEYAVLVARKKGETSASVTVATPLSDEHLATLKAALA
EKTGKDVKLDVTVDPSILGGLIVKLGSRMVDASLKTKLNSIRHAM
KEVR
SEQ ID NO: 40:
Nucleotide sequence of ATP synthase subunit
b atpF_2
ATGACCGAAATGGAACTGGCTGAGCTCTGGGTCGCCATCGCCTTC
CTGGTTTTCGTAGGCCTCCTGATCTATGCGGGCGCCCACCGCGCC
ATCGTCTCCGCCCTGGATTCCCGCGGCTCGCGCATCGCCTCGGAA
CTGGAGGAGGCCCGTCGGCTCAAGGAAGAGGCCCAGAAGCTGGTG
GCCGAATTCAAGCGCAAGCAGCGCGAGGCCGAGGCCGAGGCCGAA
TCCATCGTCACCGGCGCCAAGGCCGAGGCCGAGCGCCTCGCCGCC
GAGGCCAAGGCGAAGATCGAGGATTTCGTCACCCGCCGCACCAAG
ATGGCCGAGGACAAGATCGCCCAGGCCGAGCATCAGGCTCTGGCG
GACGTGAAGTCCATCGCCGCCGAGGCGGCGGCCAAGGCGGCCGAG
GTGATCCTCGGCGCCCAGGCCACCGGCGCGGTGGCGGAGCGTCTG
CTGTCGGGCGCCATCTCCGAGGTCAAGACCAAGCTCAACTGA
SEQ ID NO: 41:
Amino acid sequence of ATP synthase subunit
b atpF_2
MTEMELAELWVAIAFLVFVGLLIYAGAHRAIVSALDSRGSRIASE
LEEARRLKEEAQKLVAEFKRKQREAEAEAESIVTGAKAEAERLAA
EAKAKIEDFVTRRTKMAEDKIAQAEHQALADVKSIAAEAAAKAAE
VILGAQATGAVAERLLSGAISEVKTKLN
SEQ ID NO: 42:
Nucleotide sequence of ATP synthase subunit
b′ atpG_3
ATGATGATTGCATGGAAGCGGACCTTCGCAGTCGTGACCTTCGGG
GCCGCCCTGATGGCCATGCCCGTCGCGGGCGTGGTCGCAGCTGAG
ACTTCTCCCGCTCCGGCGGCAGTGGCGCAGGCCGATCATGCGGTG
CCCACCGAGGCGGCCGGCCAGGGCACCGCCGATGCGGCCCATGCC
GCCGCGCCGGGCGAGGCCGCCCATGGTGGCGCGGCCAAGCACGAA
ACCCATTTCCCGCCCTTCGACGGCACCACCTTCGCCTCCCAGTTG
CTGTGGCTCGCCGTCACCTTCGGCCTGCTTTACTACCTCATGAGC
AAGGTCACGCTGCCGCGCATCGGCCGCATCCTGGAAGAGCGCCAC
GACCGCATCGCCGATGATCTGGAGGAAGCCTCCAAGCATCGCGCC
GAGAGCGAGGCCGCCCAGCGGGCCTATGAGAAGGCGCTGAGCGAG
GCCCGCGCGAAGGCCCATTCCATCGCCGCGGAAACCCGCGACCGC
CTTGCCGCCCACGCCGACACCAACCGCAAGGCGCTGGAGAGCGAG
CTCACCGCCAAGCTGCAGGCGGCCGAGGAGCGCATCGCCACCACC
AAGAGCGAAGCCCTCACCCATGTGCGCGGCATCGCGGTGGACGCC
ACCCAATCCATCGTCTCCACCCTCATCGGTGTCGCGCCCGCGGCG
GCCGACGTGGAAAAAGCGGTGGACGGCGCCCTGTCCCAGCACGGC
CAGGCCTGA
SEQ ID NO: 43:
Amino acid sequence of ATP synthase subunit
b′ atpG_3
MMIAWKRTFAVVTFGAALMAMPVAGVVAAETSPAPAAVAQADHAV
PTEAAGQGTADAAHAAAPGEAAHGGAAKHETHFPPFDGTTFASQL
LWLAVTFGLLYYLMSKVTLPRIGRILEERHDRIADDLEEASKHRA
ESEAAQRAYEKALSEARAKAHSIAAETRDRLAAHADTNRKALESE
LTAKLQAAEERIATTKSEALTHVRGIAVDATQSIVSTLIGVAPAA
ADVEKAVDGALSQHGQA
SEQ ID NO: 44:
Nucleotide sequence of ATP synthase subunit
c atpE_2
ATGGAAGCGGAAGCTGGAAAGTTCATCGGTGCCGGCCTCGCCTGC
CTCGGCATGGGTCTCGCTGGCGTCGGCGTCGGTAACATCTTCGGT
AACTTCCTCTCCGGCGCCCTGCGCAACCCGTCCGCTGCCGACGGC
CAGTTCGCCCGCGCCTTCATCGGCGCCGCCCTCGCGGAAGGTCTC
GGCATCTTCTCGCTGGTCGTTGCGCTCGTCCTGCTGTTCGTGGCC
TGA
SEQ ID NO: 45:
Amino acid sequence of ATP synthase subunit
c atpE_2
MEAEAGKFIGAGLACLGMGLAGVGVGNIFGNFLSGALRNPSAADG
QFARAFIGAALAEGLGIFSLVVALVLLFVA
SEQ ID NO: 46:
Nucleotide sequence of ATP synthase subunit
a atpB_2
ATGACCGTCGATCCGATCCACCAGTTCGAGATCAAGCGCTACGTG
GATCTGCTGAACGTCGGCGGTGTCCAGTTCTCCTTCACCAACGCA
ACGGTGTTCATGATTGGCATCGTCCTGGTGATTTTCTTCTTCCTG
ACTTTCGCGACACGCGGTCGCACCCTTGTGCCGGGCCGGATGCAG
TCGGCGGCGGAGCTGAGCTACGAGTTCATCGCCAAGATGGTGCGC
GACGCGGCCGGCAGCGAGGGAATGGTGTTCTTTCCCTTCGTCTTC
TCGCTCTTCATGTTCGTGCTGGTGGCGAACGTATTGGGGCTCATC
CCCTACACCTTCACGGTGACCGCCCACCTCATCGTCACCGCCGCC
CTGGCGGCGACGGTGATCCTCACCGTCATCATCTACGGCTTCGTG
CGGCACGGCACCCACTTCCTGCACCTGTTCGTGCCGTCGGGCGTG
CCGGGCTTCCTCCTGCCCTTCCTCGTGGTGATCGAGGTGGTGTCG
TTCCTGTCGCGGCCCATCAGCCTCTCGCTGCGTCTGTTCGCCAAC
ATGCTGGCGGGCCACATCGCCCTCAAGGTGTTCGCCTTCTTCGTC
GTGGGACTGGCCTCGGCCGGCGCGATCGGCTGGTTCGGCGCCACC
CTGCCCTTCTTCATGATCGTGGCGCTCACCGCGCTGGAGCTGCTG
GTGGCGGTGCTGCAGGCCTACGTGTTCGCGGTGCTGACCTCGATC
TACCTCAACGACGCCATCCATCCCGGCCACTGA
SEQ ID NO: 47:
Amino acid sequence of ATP synthase subunit
a atpB_2
MTVDPIHQFEIKRYVDLLNVGGVQFSFTNATVFMIGIVLVIFFFL
TFATRGRTLVPGRMQSAAELSYEFIAKMVRDAAGSEGMVFFPFVF
SLFMFVLVANVLGLIPYTFTVTAHLIVTAALAATVILTVIIYGFV
RHGTHFLHLFVPSGVPGFLLPFLVVIEVVSFLSRPISLSLRLFAN
MLAGHIALKVFAFFVVGLASAGAIGWFGATLPFFMIVALTALELL
VAVLQAYVFAVLTSIYLNDAIHPGH
SEQ ID NO: 48:
Nucleotide sequence of ATP synthase protein
I atpI
ATGTCCGAGCCGAATGATCCATCCCGCAGGGACGGTGCGAAGGCG
AAAGACGAGACGCAGGACTCCCGGCCCGGTGAGGCGGATCTTGCT
CGGCGCCTCGATGCGCTCGGCACCTCCATCGGTCAGGTCAAGTCC
AGAAGCGGGGAGCCCGCGGCGACGCCGCGCAAGGACACCTCCTCG
GCCTCCGGCGCGGCCCTGGCGTTTCGGCTGGGCGCCGAGTTTGTT
TCAGGCGTGCTGGTGGGCTCGCTCATCGGCTACGGGTTGGATTAT
GCGTTTGCGATTTCGCCCTGGGGGCTGATCGCCTTCACGCTGATC
GGCTTTGCCGCCGGCGTCCTGAACATGCTGCGCGTGGCGAACAGC
GATGCCAAGCGCCACAGCGCGGACAGGTGA
SEQ ID NO: 49:
Amino acid sequence of ATP synthase protein
I atpI
MSEPNDPSRRDGAKAKDETQDSRPGEADLARRLDALGTSIGQVKS
RSGEPAATPRKDTSSASGAALAFRLGAEFVSGVLVGSLIGYGLDY
AFAISPWGLIAFTLIGFAAGVLNMLRVANSDAKRHSADR

In another embodiment, the bacterial strain used in the process of the invention comprises a gene encoding a nitrogenase molybdenum-iron protein alpha chain nifD_1 having the sequence set forth in SEQ ID NO:51 or a sequence having more than 60% identity, e.g. more than 70% identity, such as more than 92% identity, such as more than 95% identity, e.g. more than 96% identity, such as more than 97% identity, e.g. more than 98%, such as more than 99% sequence identity to the sequence set forth in SEQ ID NO:51.

In another embodiment, the bacterial strain used in the process of the invention comprises a gene encoding nitrogenase molybdenum-iron protein alpha chain nifD_2 having the sequence set forth in SEQ ID NO:53 or a sequence having more than 60% identity, e.g. more than 98%, such as more than 99% sequence identity to the sequence set forth in SEQ ID NO:53.

In another embodiment, the bacterial strain used in the process of the invention comprises a gene encoding a nitrogenase molybdenum-iron protein beta chain nifK_1 having the sequence set forth in SEQ ID NO:55 or a sequence having more than 87% identity, e.g. more than 90% identity, such as more than 95% identity, e.g. more than 96% identity, such as more than 97% identity, e.g. more than 98%, such as more than 99% sequence identity to the sequence set forth in SEQ ID NO:55.

In another embodiment, the bacterial strain used in the process of the invention comprises a gene encoding a nitrogenase molybdenum-iron protein beta chain nifK_2 having the sequence set forth in SEQ ID NO:57 or a sequence having more than 95% identity, e.g. more than 96% identity, such as more than 97% identity, e.g. more than 98%, such as more than 99% sequence identity to the sequence set forth in SEQ ID NO:57.

In another embodiment, the bacterial strain used in the process of the invention comprises a gene encoding a nitrogenase iron protein nifH having the sequence set forth in SEQ ID NO:59 or a sequence having more than 98.5% sequence identity to the sequence set forth in SEQ ID NO:59.

SEQ ID NO: 50:
Nucleotide sequence of Nitrogenase
molybdenum-iron protein alpha chain nifD_1
ATGAGTTCGCTCTCCGCCACTATTCAACAGGTCTTCAACGAGCCG
GGCTGCGCGAAGAACCAGAATAAGTCCGAGGCGGAGAAGAAGAAG
GGCTGCACCAAGCAGCTGCAACCCGGCGGAGCGGCCGGCGGCTGC
GCGTTCGACGGCGCGAAGATCGCGCTCCAGCCCTTGACCGACGTC
GCCCACCTGGTGCACGGCCCCATCGCCTGCGAAGGCAATTCCTGG
GACAATCGTGGCGCCAAGTCCTCCGGCTCGAACATCTGGCGCACC
GGCTTCACCACGGACATCAACGAAACCGACGTGGTGTTCGGCGGC
GAGAAGCGTCTGTTCAAGTCCATCAAGGAAATCATCGAGAAGTAC
GACCCGCCGGCCGTCTTCGTCTATCAGACCTGCGTCCCCGCCATG
ATCGGCGACGACATCGACGCGGTGTGCAAGGCGGCCAGGGAGAAG
TTCGGAAAGCCGGTGATCCCGATCAATTCCCCCGGCTTCGTGGGG
CCGAAGAATCTCGGCAACAAGCTCGCCGGCGAGGCGCTCCTCGAC
CATGTGATCGGCACCGAGGAGCCCGATTACACGACGGCCTACGAC
ATCAACATCATCGGCGAATACAATCTCTCCGGCGAGTTGTGGCAG
GTGAAGCCGCTGCTGGACGAGCTGGGCATCCGCATCCTCGCCTGC
ATCTCCGGCGACGGGAAGTACAAGGATGTGGCGTCCTCCCACCGC
GCCAAGGCGGCGATGATGGTGTGCTCCAAGGCCATGATCAACGTG
GCCCGCAAGATGGAGGAGCGCTACGACATCCCCTTCTTCGAAGGC
TCCTTCTACGGCATCGAGGATAGCTCCGATTCCCTGCGCGAGATT
GCGCGCATGCTCATCGAGAAGGGCGCCGATCCGGAGCTGATGGAC
CGCACCGAGGCGCTGATTGAGCGGGAAGAGAAGAAGGCGTGGGAC
GCCATCGCCGCCTACAAGCCCCGCTTCAAGGACAAGAAGGTGCTG
CTCATCACCGGCGGCGTGAAATCCTGGTCGGTGGTGGCAGCGCTC
CAGGAAGCCGGCCTCGAACTGGTGGGCACCTCGGTGAAGAAGTCC
ACCAAGGAGGACAAGGAGCGCATCAAGGAACTGATGGGCCAGGAC
GCCCACATGATCGACGACATGACGCCCCGCGAAATGTACAAGATG
CTGAAGGACGCCAAGGCGGACATCATGCTCTCGGGCGGGCGCTCG
CAATTCATCGCGCTCAAGGCCGCCATGCCCTGGCTCGACATCAAC
CAGGAGCGCCACCACGCCTATATGGGCTATGTGGGCATGGTGAAG
CTGGTCGAGGAGATCGACAAGGCGCTCTACAATCCCGTGTGGGAA
CAGGTGCGCAAGCCCGCCCCGTGGGAAAATCCGGAAGACACCTGG
CAGGCCCGTGCGCTCGCCGAAATGGAGGCGGAGGCCGCCGCGCTC
GCCGCCGATCCGGTGCGCGCGGAAGAGGTGCGCCGGTCCAAGAAG
ATCTGCAATTGCAAGAGCGTCGACCTCGGAACCATTGAGGACGCC
ATCAAGGCTCACGCGCTGACCACCGTGGAGGGTGTGCGAGAGCAC
ACCAATGCCTCGGGAGGCTGCGGAGCCTGCAGCGGGCGGATCGAG
GAGATCTTCGAGGCCGTGGGCGTTGTCGCCGCCCCGCCTCCCGCG
GAGGCCGCCCCGTCTCCGCAGGAGATCGCGCCCGATCCGCTCGCT
GCGGAGGAAAAGCGCCGCGCCAAGAAGGCCTGCGGCTGCAAGGAG
GTAGCGGTCGGCACCATTGAGGATGCCATCCGCGCCAAGGGTCTG
CGAAACATCGCGGAGGTGCGTGCGGCCACCGATGCCAACACCGGC
TGCGGCAATTGCCAGGAGCGGGTGGAGGGCATCCTCGACCGGGTT
CTCGCCGAGGCGGCCTCAGAACTCCAGGCGGCGGAATAG
SEQ ID NO: 51:
Amino acid sequence of Nitrogenase
molybdenum-iron protein alpha chain nifD_1
MSSLSATIQQVFNEPGCAKNQNKSEAEKKKGCTKQLQPGGAAGGC
AFDGAKIALQPLTDVAHLVHGPIACEGNSWDNRGAKSSGSNIWRT
GFTTDINETDVVFGGEKRLFKSIKEIIEKYDPPAVFVYQTCVPAM
IGDDIDAVCKAAREKFGKPVIPINSPGFVGPKNLGNKLAGEALLD
HVIGTEEPDYTTAYDINIIGEYNLSGELWQVKPLLDELGIRILAC
ISGDGKYKDVASSHRAKAAMMVCSKAMINVARKMEERYDIPFFEG
SFYGIEDSSDSLREIARMLIEKGADPELMDRTEALIEREEKKAWD
AIAAYKPRFKDKKVLLITGGVKSWSVVAALQEAGLELVGTSVKKS
TKEDKERIKELMGQDAHMIDDMTPREMYKMLKDAKADIMLSGGRS
QFIALKAAMPWLDINQERHHAYMGYVGMVKLVEEIDKALYNPVWE
QVRKPAPWENPEDTWQARALAEMEAEAAALAADPVRAEEVRRSKK
ICNCKSVDLGTIEDAIKAHALTTVEGVREHTNASGGCGACSGRIE
EIFEAVGVVAAPPPAEAAPSPQEIAPDPLAAEEKRRAKKACGCKE
VAVGTIEDAIRAKGLRNIAEVRAATDANTGCGNCQERVEGILDRV
LAEAASELQAAE
SEQ ID NO: 52:
Nucleotide sequence of Nitrogenase
molybdenum-iron protein alpha chain nifD_2
ATGAGTGTCGCACAGTCCCAGAGCGTCGCCGAGATCAAGGCGCGC
AACAAGGAACTCATCGAAGAGGTCCTCAAGGTCTATCCCGAGAAG
ACCGCCAAGCGCCGCGCCAAGCACCTGAACGTCCACGAAGCCGGC
AAGTCCGACTGCGGCGTGAAGTCCAACATCAAGTCCATCCCGGGC
GTGATGACCATCCGCGGTTGCGCTTATGCCGGCTCCAAGGGTGTG
GTGTGGGGTCCCATCAAGGACATGATCCACATCTCCCACGGCCCG
GTGGGCTGCGGCCAGTATAGCTGGGCCGCCCGCCGCAACTACTAT
ATCGGCACGACCGGCATCGACACCTTCGTGACGATGCAGTTCACC
TCCGACTTCCAGGAGAAGGACATCGTCTTCGGCGGCGACAAGAAG
CTCGCCAAGATCATGGACGAGATCCAGGAGCTGTTCCCGCTGAAC
AACGGCATCACCGTTCAGTCCGAGTGCCCCATCGGCCTCATCGGC
GACGACATCGAGGCCGTCTCCAAGCAGAAGTCCAAGGAGTATGAG
GGCAAGACCATCGTGCCGGTGCGCTGCGAGGGCTTCCGCGGCGTG
TCCCAGTCCCTGGGCCACCACATCGCCAACGACGCCATCCGCGAT
TGGGTGTTCGACAAGATCGCGCCCGACGCCGAGCCGCGCTTTGAG
CCGACCCCGTACGACGTCGCCATCATCGGCGACTACAATATCGGT
GGTGACGCCTGGTCGTCCCGTATCCTCCTGGAGGAGATGGGCCTG
CGCGTGATCGCCCAGTGGTCCGGCGACGGTTCGCTCGCTGAGCTG
GAGGCCACCCCGAAGGCCAAGCTCAACGTGCTGCACTGCTACCGC
TCCATGAACTACATCTCGCGCCACATGGAAGAGAAGTACGGTATC
CCGTGGTGCGAGTACAACTTCTTCGGTCCTTCCAAGATCGCCGAG
TCCCTGCGCAAGATCGCCAGCTACTTCGACGACAAGATCAAGGAA
GGCGCGGAGCGCGTCATCGCCAAGTATCAGCCGCTCATGGATGCG
GTGATCGCGAAGTATCGTCCCCGCCTCGAGGGCAAGACCGTGATG
CTGTACGTGGGCGGCCTGCGTCCCCGTCACGTCATCGGCGCCTAC
GAGGACCTGGGCATGGAAGTGGTCGGCACGGGCTACGAGTTCGCC
CATAACGACGACTACCAGCGCACCGCCCAGCACTACGTCAAGGAT
GGCACCATCATCTATGACGACGTGACCGGCTACGAGTTCGAGAAG
TTCGTCGAGAAGATCCAGCCGGACCTGGTCGGTTCGGGCATCAAG
GAAAAGTACGTCTTCCAGAAGATGGGCGTGCCGTTCCGCCAGATG
CACTCCTGGGACTACTCGGGCCCGTACCACGGCTATGACGGCTTC
GCGATCTTCGCGCGCGACATGGACATGGCCATCAACAGCCCCGTG
TGGAAGATGACCCAGGCTCCGTGGAAGAGCGTCCCCAAGCCGACG
ATGCTCGCGGCTGAATGA
SEQ ID NO: 53:
Amino acid sequence of Nitrogenase
molybdenum-iron protein alpha chain nifD_2
MSVAQSQSVAEIKARNKELIEEVLKVYPEKTAKRRAKHLNVHEAG
KSDCGVKSNIKSIPGVMTIRGCAYAGSKGVVWGPIKDMIHISHGP
VGCGQYSWAARRNYYIGTTGIDTFVTMQFTSDFQEKDIVFGGDKK
LAKIMDEIQELFPLNNGITVQSECPIGLIGDDIEAVSKQKSKEYE
GKTIVPVRCEGFRGVSQSLGHHIANDAIRDWVFDKIAPDAEPRFE
PTPYDVAIIGDYNIGGDAWSSRILLEEMGLRVIAQWSGDGSLAEL
EATPKAKLNVLHCYRSMNYISRHMEEKYGIPWCEYNFFGPSKIAE
SLRKIASYFDDKIKEGAERVIAKYQPLMDAVIAKYRPRLEGKTVM
LYVGGLRPRHVIGAYEDLGMEVVGTGYEFAHNDDYQRTAQHYVKD
GTIIYDDVTGYEFEKFVEKIQPDLVGSGIKEKYVFQKMGVPFRQM
HSWDYSGPYHGYDGFAIFARDMDMAINSPVWKMTQAPWKSVPKPT
MLAAE
SEQ ID NO: 54:
Nucleotide sequence of Nitrogenase
molybdenum-iron protein beta chain nifK_1
ATGGCCACCGTTTCCGTCTCCAAGAAGGCCTGCGCGGTCAACCCC
CTCAAGATGAGCCAGCCGGTGGGCGGCGCGCTCGCCTTCATGGGC
GTGCGCAAGGCCATGCCGCTGCTGCACGGCTCGCAGGGCTGCACC
TCCTTCGGCCTGGTGCTGTTCGTGCGCCACTTCAAGGAAGCCATC
CCCATGCAGACCACCGCCATGAGCGAGGTGGCGACGGTTCTGGGC
GGCCTTGAGAATGTGGAGCAGGCCATTCTCAACATCTACAATCGC
ACCAAGCCGGAGATCATCGGCATCTGCTCCACCGGCGTCACCGAG
ACCAAGGGCGATGATGTCGACGGCTACATCAAGCTGATCCGGGAC
AAGTATCCCCAGCTGGCCGACTTCCCGCTGGTCTATGTCTCCACC
CCCGATTTCAAGGACGCCTTCCAGGACGGTTGGGAGAAGACCGTG
GCGAAGATGGTGGAGGCGCTGGTGAAGCCCGCCGCCGACAAGCAG
AAGGACAAGACCCGCGTCAACGTCCTGCCCGGCTGCCACCTCACG
CCCGGCGATCTGGATGAGATGCGGACCATCTTCGAGGATTTCGGG
CTCACACCCTATTTCCTGCCGGATCTGGCCGGCTCGCTGGATGGG
CATATCCCCGAGGACTTCTCGCCCACCACCATCGGCGGCATCGGC
ATCGATGAGATCGCCACCATGGGCGAGGCGGCCCACACCATCTGC
ATCGGCGCGCAGATGCGCCGGGCGGGCGAGGCCATGGAGAAGAAG
ACCGGCATTCCCTTCAAGCTGTTCGAGCGCCTGTGCGGCCTGGAG
GCGAACGACGCCTTCATCATGCACCTGTCGCAGATCTCCGGCCGG
CCGGTGCCGGTGAAGTATCGCCGGCAGCGGGGCCAGCTGGTGGAT
GCCATGCTGGACGGCCACTTCCATCTGGGCGGTCGCAAGGTGGCC
ATGGGGGGGGAGCCGGACCTGCTCTACGACGTGGGCTCCTTCCTG
CACGAGATGGGCGCCCACATCCTTTCCGCGGTCACCACCACCCAG
TCGCCGGTGCTGGCGCGCCTGCCTGCCGAGGAGGTGCTTATCGGC
GACCTGGAGGATCTGGAGACCCAGGCGAAGGCGCGCGGATGCGAT
CTCCTGCTCACCCATTCCCATGGGCGCCAGGCGGCGGAGCGCCTC
CACATCCCCTTCTACCGGATCGGCATTCCCATGTTTGACCGGCTG
GGGGCGGGGCATCTGTTGTCGGTGGGCTATCGCGGCACCCGCGAC
CTCATCTTCCATCTCGCCAACCTTGTGATCGCCGACCACGAGGAA
AATCACGAGCCGACGCCCGACACCTGGGCCACCGGCCATGGCGAG
CATGCCGCCGCCCCCACTTCCCATTGA
SEQ ID NO: 55:
Amino acid sequence of Nitrogenase
molybdenum-iron protein beta chain nifK_1
MATVSVSKKACAVNPLKMSQPVGGALAFMGVRKAMPLLHGSQGCT
SFGLVLFVRHFKEAIPMQTTAMSEVATVLGGLENVEQAILNIYNR
TKPEIIGICSTGVTETKGDDVDGYIKLIRDKYPQLADFPLVYVST
PDFKDAFQDGWEKTVAKMVEALVKPAADKQKDKTRVNVLPGCHLT
PGDLDEMRTIFEDFGLTPYFLPDLAGSLDGHIPEDFSPTTIGGIG
IDEIATMGEAAHTICIGAQMRRAGEAMEKKTGIPFKLFERLCGLE
ANDAFIMHLSQISGRPVPVKYRRQRGQLVDAMLDGHFHLGGRKVA
MGAEPDLLYDVGSFLHEMGAHILSAVTTTQSPVLARLPAEEVLIG
DLEDLETQAKARGCDLLLTHSHGRQAAERLHIPFYRIGIPMFDRL
GAGHLLSVGYRGTRDLIFHLANLVIADHEENHEPTPDTWATGHGE
HAAAPTS H
SEQ ID NO: 56:
Nucleotide sequence of Nitrogenase
molybdenum-iron protein beta chain nifK_2
ATGCCACAAAATGCTGACAATGTGCTCGATCACTTCGAGCTCTTC
CGTGGTCCCGAATACCAGCAGATGCTGGCCAATAAGAAAAAGATG
TTCGAGAACCCCCGCGATCCGGCCGAAGTCGAGCGCGTGCGGGAA
TGGGCGAAGACTCCTGAATACAAGGAGCTGAACTTCGCCCGCGAG
GCGCTCACCGTGAATCCGGCCAAGGCTTGTCAGCCGCTGGGCGCG
GTGTTCGTCGCCGTCGGCTTCGAGAGCACGATCCCCTTCGTGCAC
GGCTCGCAGGGTTGCGTCGCGTATTACCGCTCGCACCTCTCCCGC
CACTTCAAGGAGCCGTCCTCCTGCGTCTCCTCGTCCATGACCGAG
GATGCGGCGGTGTTCGGCGGCCTCAACAACATGATTGACGGCCTC
GCCAACACCTACAACATGTACAAGCCGAAGATGATCGCCGTCTCC
ACCACCTGCATGGCGGAAGTCATCGGCGACGATCTGAACGCCTTC
ATCAAGACCGCGAAGGAAAAGGGCTCGGTTCCGGCCGAATACGAC
GTGCCCTTCGCCCACACCCCGGCGTTCGTCGGCAGCCATGTCACC
GGCTACGACAATGCGCTCAAGGGCATCCTCGAGCACTTCTGGGAC
GGCAAGGCCGGCACCGCGCCGAAGCTGGAGCGCGTTCCCAACGAG
AAGATCAACTTCATCGGCGGCTTCGACGGCTACACCGTCGGCAAC
ACTCGCGAAGTGAAGCGCATCTTCGAGGCGTTCGGCGCCGATTAC
ACCATCCTCGCCGACAATTCCGAAGTGTTCGACACCCCGACCGAC
GGCGAGTTCCGCATGTATGACGGCGGCACGACCCTGGAGGACGCG
GCGAACGCGGTGCACGCCAAGGCCACCATCTCCATGCAGGAATAC
TGCACGGAGAAGACCCTGCCCATGATCGCCGGTCATGGCCAGGAC
GTGGTCGCCCTCAACCACCCCGTGGGCGTGGGCGGCACCGACAAG
TTCCTCATGGAGATCGCCCGCCTCACCGGCAAGGAGATCCCCGAG
GAGCTGACCCGCGAGCGCGGCCGTCTCGTGGACGCTATCGCGGAC
TCTTCCGCGCACATCCACGGCAAGAAGTTCGCCATCTACGGCGAT
CCGGATCTGTGCCTGGGCCTCGCCGCGTTCCTGCTGGAGCTGGGC
GCCGAGCCGACCCATGTGCTGGCCACCAACGGCACCAAGAAGTGG
GCCGAGAAGGTTCAGGAACTGTTCGACTCTTCGCCGTTCGGCGCC
AACTGCAAGGTCTATCCCGGCAAGGACCTGTGGCACATGCGCTCG
CTCCTGTTCGTGGAGCCGGTGGATTTCATCATCGGCAACACCTAC
GGCAAGTATCTCGAGCGCGACACGGGCACCCCGCTGATCCGTATC
GGCTTCCCGGTGTTCGACCGTCACCACCACCACCGCCGTCCGGTG
TGGGGCTATCAGGGCGGCATGAACGTCCTGATCACGATCCTCGAC
AAGATCTTTGACGAGATCGACCGCAACACCAACGTGCCGGCCAAG
ACCGACTACTCGTTCGACATCATTCGTTGA
SEQ ID NO: 57:
Amino acid sequence of Nitrogenase
molybdenum-iron protein beta chain nifK_2
MPQNADNVLDHFELFRGPEYQQMLANKKKMFENPRDPAEVERVRE
WAKTPEYKELNFAREALTVNPAKACQPLGAVFVAVGFESTIPFVH
GSQGCVAYYRSHLSRHFKEPSSCVSSSMTEDAAVFGGLNNMIDGL
ANTYNMYKPKMIAVSTTCMAEVIGDDLNAFIKTAKEKGSVPAEYD
VPFAHTPAFVGSHVTGYDNALKGILEHFWDGKAGTAPKLERVPNE
KINFIGGFDGYTVGNTREVKRIFEAFGADYTILADNSEVFDTPTD
GEFRMYDGGTTLEDAANAVHAKATISMQEYCTEKTLPMIAGHGQD
VVALNHPVGVGGTDKFLMEIARLTGKEIPEELTRERGRLVDAIAD
SSAHIHGKKFAIYGDPDLCLGLAAFLLELGAEPTHVLATNGTKKW
AEKVQELFDSSPFGANCKVYPGKDLWHMRSLLFVEPVDFIIGNTY
GKYLERDTGTPLIRIGFPVFDRHHHHRRPVWGYQGGMNVLITILD
KIFDEIDRNTNVPAKTDYSFDIIR
SEQ ID NO: 58:
Nucleotide sequence of Nitrogenase
iron protein nifH
GTGGAGTCCGGTGGTCCTGAGCCGGGCGTGGGCTGCGCCGGCCGC
GGCGTGATCACCTCCATCAACTTCCTGGAGGAGAACGGCGCCTAC
GAGGACATCGACTATGTGTCCTACGACGTGCTGGGCGACGTGGTG
TGCGGCGGCTTCGCCATGCCCATCCGCGAGAACAAGGCGCAGGAA
ATCTACATCGTGATGTCCGGCGAGATGATGGCCATGTATGCGGCC
AACAACATCTCCAAGGGCATCCTGAAGTATGCCAATTCCGGCGGC
GTGCGCCTGGGCGGGCTGGTCTGCAACGAGCGCCAGACCGACAAG
GAGCTGGAGCTGGCGGAGGCTCTGGCGAAGAAGCTCGGCACCGAG
CTGATCTACTTCGTGCCGCGCGACAACATCGTGCAGCATGCCGAG
CTGCGCCGCATGACAGTGATCGAGTATGCGCCCGATTCCGCCCAG
GCCCAGCACTACCGGAACCTGGCCGAGAAGGTGCACGCCAACAAG
GGCAACGGCATCATCCCGACCCCGATCACCATGGACGAGCTGGAA
GACATGCTCATGGAGCACGGCATCATGAAGGCCGTGGACGAGAGC
CAGATCGGCAAGACCGCCGCCGAGCTCGCCGTCTGA
SEQ ID NO: 59:
Amino acid sequence of Nitrogenase
iron protein nifH
MESGGPEPGVGCAGRGVITSINFLEENGAYEDIDYVSYDVLGDVV
CGGFAMPIRENKAQEIYIVMSGEMMAMYAANNISKGILKYANSGG
VRLGGLVCNERQTDKELELAEALAKKLGTELIYFVPRDNIVQHAE
LRRMTVIEYAPDSAQAQHYRNLAEKVHANKGNGIIPTPITMDELE
DMLMEHGIMKAVDESQIGKTAAELAV

Downstream Processing

In one embodiment, the process of the invention comprises the further step of harvesting biomass produced during the culture. Biomass can e.g. be harvested by sedimentation (settling based on gravity), filtration, centrifugation or flocculation. Flocculation may require the addition of a flocculation agent. Centrifugation may e.g. be carried out using a continuous flow centrifuge.

In one embodiment, the harvested biomass is subsequently dried. Drying can e.g. be performed using well known methods, including centrifugation, drum drying, evaporation, freeze drying, heating, spray drying, vacuum drying and/or vacuum filtration. The dried biomass may subsequently be used in a product, e.g. a food or feed product or feed or food ingredient.

In another embodiment, the cells of the harvested biomass are lysed. The lysate may in some embodiments be separated into insoluble and soluble fractions, either or both of which may subsequently be concentrated or dried, and subsequently be used in a product, e.g. a food or a feed product.

In one embodiment, biomass is harvested and proteins are isolated from said biomass, resulting in a protein fraction and a fraction comprising non-protein components. Thus, in one embodiment, the process is for the production of protein and comprises a step of culturing strain VTT-E-193585 or a derivative thereof, followed by a step of harvesting biomass and a further step of isolating proteins from said biomass. In another embodiment, the process is for the production of protein and comprises culturing a bacterial strain of the genus Xanthobacter in continuous culture with hydrogen as energy source and an inorganic carbon source, wherein the inorganic carbon source comprises carbon dioxide, followed by a step of harvesting biomass and a further step of isolating proteins from said biomass. Depending on the method of protein isolation, the resulting fractions may be more pure or less pure. Thus, the term “protein fraction” means a fraction enriched in proteins. The protein fraction may still comprise significant amounts of other components and also significant amounts of protein may end up in the “fraction comprising non-protein components”.

Isolation of proteins may be performed using any suitable method. For example, in one embodiment, proteins are isolated by breaking cells mechanically and separating protein from cell debris through one or more filtration steps, e.g. successive filtration through multiple filters with decreasing pore size. Mechanical breaking may be carried out using any suitable method, e.g. ball milling, sonication, homogenization, high pressure homogenization, mechanical shearing, etc. The resulting filtered protein fraction will be enriched in proteins, but also still contain other smaller components. Protein may optionally be further purified from this fraction using any suitable method.

In another embodiment, a protein fraction is isolated by performing ethanol extraction followed by one or more filtration steps. Such methods are e.g. known from the preparation of soy bean proteins (see e.g. Chapter 5 “Soybean Protein Concentrates” in “Technology of production of edible flours and protein products from soybeans” by Berk FAO Agricultural Services Bulletin No. 97 (1992). The resulting protein fraction will be enriched in proteins, but also still contain other components. Protein may optionally be further purified from this fraction using any suitable method.

In one embodiment, the process of the invention comprises the further step of hydrolysing the protein fraction obtained from the process of the invention to obtain amino acids and small peptides.

In one embodiment of the process of the invention, the process comprises the further step of producing a food or feed product from said biomass, from said protein fraction or from said fraction comprising non-protein components. Said further step may simply comprise incorporating said biomass, protein fraction or fraction comprising non-protein components in a food or feed product, by adding it during the production of the food or feed product. In other embodiments, further purification or modification of the biomass or fraction thereof is performed during the course of its incorporation into a food or feed product.

In a further aspect, the invention relates to a product, such as biomass, protein, or non-protein components, obtained or obtainable by the process according to the invention.

In one embodiment, the product obtained from the process of the invention comprises more than 40% protein, such as between 40% and 99% protein, e.g. between 40% and 90% protein, such as between 40% and 60% protein. In a particular embodiment, the product comprises between 25% and 75% protein, between 0% and 20% lipid and between 5% and 40% carbohydrates. In a further embodiment, the product comprises between 40% and 60% protein, between 0% and 15% lipid and between 10% and 25% carbohydrate. In an even further embodiment, the product obtained from the process of the invention comprises between 45% and 55% protein, between 5% and 10% lipid and between 10% and 20% carbohydrates.

As described above, the invention in a further aspect relates to a food or feed product obtained or obtainable by the process according to the invention. When used herein, the terms “food” and “feed” are intended to include not only conventional food and feed products, such as processed foods, but also related products, such as food and feed supplements, e.g. protein bars, powders or shakes, meat replacements, food ingredients, probiotics, prebiotics, nutraceuticals and the like. In certain embodiments, said biomass, said protein fraction or said fraction comprising non-protein components is utilized in the production of a vegetarian or vegan food product.

The invention is further illustrated with the following, non-limiting, examples:

### EXAMPLES

### Example 1. Isolation of Bacterial Strain Capable of Chemoautotrophic Growth

A sample of 50 mL containing soil and seawater was collected in a sterile falcon tube from the seashore of the Baltic sea in Naantali in Finland. Part of soil sample was mixed with 10 mL of mineral medium in a sterile Erlenmeyer flask. The medium consisted of 1 g/L NH₄OH, 0.23 g/L KH₂PO₄, 0.29 g/L Na₂HPO₄·2 H₂O, 0.005 g/L NaVO₃·H₂O, 0.2 g/L FeSO₄·7 H₂O, 0.5 g/L MgSO₄·7 H₂O, 0.01 g/L CaSO₄, 0.00015 g/L Na₂MoO₄·2 H₂O, 0.005 g/L MnSO₄, 0.0005 g/L ZnSO₄·7 H₂O, 0.0015 g/L H₃BO₃, 0.001 g/L CoSO₄, 0.00005 g/L CuSO₄ and 0.0001 g/L NiSO₄ prepared in tap water. The suspension of soil and medium was incubated in a shaking incubator in +30° C. temperature in a sealed steel box that was flushed continuously with a gas mixture: 150 mL/min of N₂, 18 mL/min of H₂, 3 mL/min of O₂ and 6 mL/min of CO₂. The cultivation was refreshed in seven-day intervals by taking 1 mL of suspension, which was added in sterile conditions to 9 mL of medium in Erlenmeyer flask, and then placed back into the incubation box. After the fourth dilution, there was no noticeable soil left in the suspension. The volume of the cell suspension was increased to 100 mL in order to grow biomass for bioreactor cultivation. The optical density (OD₆₀₀) of the suspension was 1.53 when it was inoculated to 190 mL of mineral medium in 15-vessel 200-mL parallel bioreactor system (Medicel Explorer, Medicel Oy, Finland). The cultivation conditions were 800 rpm agitation, +30° C. temperature and the pH was set to 6.8, controlling it with 1 M NaOH. Gas was fed through a sparger with a gas mixture consisting of 14 mL/min H₂, 3 mL/min O₂ and 6 mL/min CO₂. The head space of the reactor was flushed with 300 mL/min air. Continuous cultivation was fed with mineral medium 6 mL/h and cell suspension was drawn from the reactor via capillary keeping the volume constant at 200 mL. Cell suspension drawn from the reactor was stored at +4° C. A sample was taken from the bioreactor automatically every day, and absorbance at 600 nm was measured to monitor the growth. After 498 hours of bioreactor cultivation, samples were drawn aseptically and suspension was diluted and plated to agar mineral medium plates containing the above minerals and 2% bacteriological agar. Plates were incubated in same conditions as described above for the Erlenmeyer flasks. Colonies were then picked from agar plates and streaked to new agar plates in order to isolate one organism in one colony. This was repeated twice. Single colonies were picked and suspended into 200 μL of medium in a 96-well microtiter plate. The suspension was incubated at +30° C. temperature and shaken 625 rpm in an EnzyScreen gas tight box that was flushed continuously with 150 mL/min of N₂, 18 mL/min of H₂, 3 mL/min of O₂ and 6 mL/min of CO₂. The suspension from one well was transferred to an Erlenmeyer flask and supplemented with fresh medium. Volume was increased until there was enough biomass to perform a bioreactor cultivation. The organism was deposited in the VTT culture collection as VTT-E-193585.

16S rRNA sequencing of a sample demonstrated that the sample contained only one organism. The same sample was used for Illumina NextSeq sequencing providing 1×150 bp metagenomic shotgun sequences. Using Unicycler (Wick et al, 2017 PLoS computational biology 13:e1005595), the de novo assembly was made for metagenomic sequences consisting of 101 contigs. The total genome length was 4,846,739 bp and the GC content was 67.9%. Gene predictions and functional annotations were performed using Prokka (Seemann, 2014 Bioinformatics 30:2068). The genome annotation produced 4,429 genes. Roary pan genomic alignment (Page et al, 2015 Bioinformatics 31:3691) grouped VTT-E-193585 among Xanthobacter species. The strain was therefore identified as a Xanthobacter sp., the closest genome being Xanthobacter tagetidis. Alignment-based calculation of average nucleotide identity that takes into account only orthologous fragments (OrthoANI) (Lee et al, 2016 Int J Syst Evol Microbiol 66:1100) gave the best match of 80.4% to Xanthobacter tagetidis (ATCC 700314; GCF_003667445.1), whereas the proposed species boundary cut-off is 95-96% (see e.g., Chun et al., 2018 Int J Syst Evol Microbiol, 68: 461-466). Xanthobacter autotrophicus Py2 gave a match of 79.6%, while the match for Xanthobacter sp. 91 was 79.0%. It could thus be concluded that the isolated bacterial strain deposited as VTT-E-193585 belongs to the Phylum: Proteobacteria; to the Class: Alpha Proteobacteria; and to the Order: Rhizobiales. The most probable Family is Xanthobacteraceae, and the Genus Xanthobacter. The VTT-E-193585 bacterial strain could not be assigned unequivocally to any known species.

A search for putative antimicrobial resistance genes was performed. The ABRicate (https://github.com/tseemann/abricate) tool was used to search the genome against the Arg-Annot, NCBI, ResFinder, the ecOH, Megares and VFDB databases using blastn or blastp. A threshold of 50% was set for both identity and coverage, both on nucleotide and protein level. Only two putative antimicrobial resistance genes were identified. These two genes did not contain amino-acid changes linked to antibiotics resistance and thus a resistant phenotype is not expected.

### Example 2. Pilot Cultivation and Analysis of Isolated Bacterial Strain

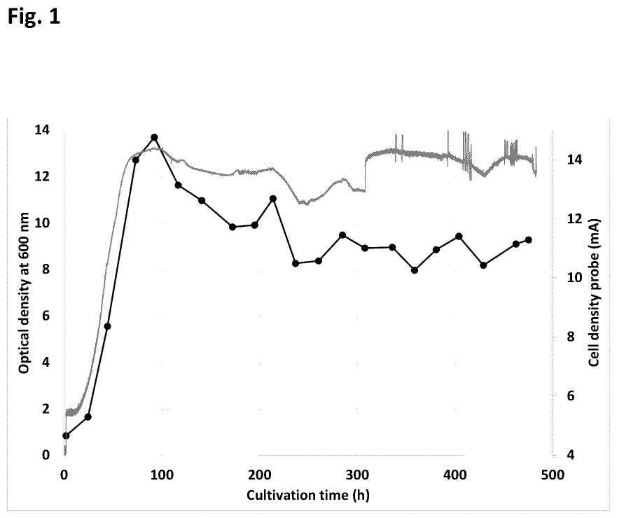

The isolated bacterial strain deposited as VTT-E-193585 was cultivated in a conventional 200-liter stirred tank bioreactor (MPF-U, Marubishi Ltd, Japan). Mixing was performed with Rushton-type impellers rotating at 400 rpm. Temperature in the cultivation was maintained at +30° C. pH was maintained at 6.8±0.2 by adding 8 M NaOH or 3.6 M H₃PO₄ by software control. Cultivation medium contained 1 g/L NH₄OH, 0.23 g/L KH₂PO₄, 0.29 g/L Na₂HPO₄·2 H₂O, 0.005 g/L NaVO₃·H₂O, 0.2 g/L FeSO₄·7 H₂O, 0.5 g/L MgSO₄. 7 H₂O, 0.01 g/L CaSO₄, 0.00015 g/L Na₂MoO₄·2 H₂O, 0.005 g/L MnSO₄, 0.0005 g/L ZnSO₄·7 H₂O, 0.0015 g/L H₃BO₃, 0.001 g/L CoSO₄, 0.00005 g/L CuSO₄ and 0.0001 g/L NiSO₄ prepared in tap water. A mixture containing 1.8-10.5 L/min hydrogen gas, 0.6-2.5 L/min oxygen gas and 1.8-5 L/min carbon dioxide gas was supplied constantly as the main source of energy and carbon. Dissolved oxygen level was maintained at 7.2±0.5% by adjusting the gas mixture composition. The inoculum for the cultivation was prepared as described in Example 1. Growth was monitored by taking samples manually and analysing the cell density as optical density by measuring absorbance at 600 nm (Ultrospec 2100 pro UV/visible spectrophotometer, Biochrom Ltd., England) and by measuring cell dry weight (CDW) by drying in oven overnight at 105° C. Optical density was also monitored by using an in situ absorbance probe (Trucell 2, Finesse Ltd, USA). A growth curve of the cultivation is presented in FIG. 1. The maximum growth rate in batch phase was 0.06 h⁻¹. The maximum cell density was 4.5 g_CDW/L at 92 h. After 92 h of cultivation, feed of fresh cultivation medium as described above was started at a dilution rate of 0.01 h⁻¹. During the continued feed, the cell density was on average 2.9 g_CDW/L. Cultivation liquid was constantly collected to a cooled (+10° C.) tank from which it was fed in 300-liter batches to a continuous centrifugal separator (BTPX-205, Alfa-Laval AB, Sweden). The cell-containing slurry collected from the separator was fed into an atmospheric double drum dryer (Buflovak 6×8 ADDD, Hebeler process solutions Llc., USA), heated with 4 bar steam and drums rotating at 3.5 rpm. This resulted in dried cell powder with approximately 96/dry matter content. Analysis results of the dried cell powder are presented in Table 1 for the proximate composition, in Table 2 for the amino acid composition, in Table 3 for the fatty acid composition, and in Table 4 for the vitamin content. Analyses demonstrate that the dried cell powder has high protein content with all the essential amino acids. It also contains more unsaturated than saturated fatty acids and a lot of B-group vitamins. Peptidoglycan content was only 0.002 mg/g_CDW and lipopolysaccharide content was 0.01 mg/g_CDW. It would be beneficial that these concentrations would be as small as possible. In comparison, in a commercial lactic acid bacteria preparation analysed at the same time, the peptidoglycan content was 0.244 mg/g_DW and the lipopolysaccharide content was 0.015 mg/g_DW. Cytotoxicity and genotoxicity assays were performed using the supernatant samples of cultivation. No cytotoxicity against HepG2 or HeLa229 human cell lines was observed. No genotoxicity against Escherichia coli WP2 trp- or CM871 uvrA recA lexA strains was observed.

TABLE 1
Analysis results of dried cell powder of isolated bacterial strain
deposited as VTT-E-193585.

| Parameter | Method | Unit | Value |
|---|---|---|---|
| Moisture | Drying at 103° C. | g/100 g | 3.3 |
| Protein | Kjeldahl (N × 6.25) | g/100 g | 72.2 |
| Fat | Weibull-Stoldt | g/100 g | 6.0 |
| Saturated fatty acids | Calculation based on Table 3 | g/100 g | 1.8 |
| Mono-unsaturated fatty acids | Calculation based on Table 3 | g/100 g | 3.8 |
| Polyunsaturated fatty acids | Calculation based on Table 3 | g/100 g | 0.4 |
| Omega 3 fatty acids | Calculation based on Table 3 | g/100 g | <0.01 |
| Omega 6 fatty acids | Calculation based on Table 3 | g/100 g | 0.4 |
| Dietary fibres | Gravimetric | g/100 g | 10.5 |
| Ash | Ashing at 550° C. | g/100 g | 5.8 |
| Glucose | HPLC-ELSD | g/100 g | <0.15 |
| Fructose | HPLC-ELSD | g/100 g | <0.1 |
| Sucrose | HPLC-ELSD | g/100 g | <0.1 |
| Lactose | HPLC-ELSD | g/100 g | <0.25 |
| Maltose | HPLC-ELSD | g/100 g | <0.2 |
| Total sugars | Calculation | g/100 g | <0.8 |
| Carbohydrates | Calculation | g/100 g | 2.2 |
| Energy | Calculation | kJ/100 g | 1572 |
| Energy | Calculation | kcal/100 g | 373 |

TABLE 2
Amino acid composition of dried cell powder of isolated
bacterial strain deposited as VTT-E-193585.

| Parameter | Method | Unit | Value |
|---|---|---|---|
| Lysine | Ion chromatography | % | 3.95 |
| Methionine | Ion chromatography | % | 1.60 |
| Cystine | Ion chromatography | % | 0.39 |
| Aspartic | Ion chromatography | % | 6.82 |
| Threonine | Ion chromatography | % | 3.47 |
| Serine | Ion chromatography | % | 2.75 |
| Glutamic | Ion chromatography | % | 8.84 |
| Proline | Ion chromatography | % | 3.14 |
| Glycine | Ion chromatography | % | 4.40 |
| Alanine | Ion chromatography | % | 6.94 |
| Valine | Ion chromatography | % | 4.96 |
| Isoleucine | Ion chromatography | % | 3.34 |
| Leucine | Ion chromatography | % | 6.08 |
| Tyrosine | Ion chromatography | % | 2.99 |
| Phenylalanine | Ion chromatography | % | 4.58 |
| Histidine | Ion chromatography | % | 1.66 |
| Arginine | Ion chromatography | % | 4.96 |
| Tryptophan | HPLC | % | 1.34 |

TABLE 3
Fatty acid composition of dried cell powder of isolated
bacterial strain deposited as VTT-E-193585.

| Parameter | Method | Unit | Value |
|---|---|---|---|
| C16:0 (Palmitic acid) | GC-MS | % | 24.8 |
| C16:1 (Palmitoleic acid) | GC-MS | % | 3.0 |
| C18:0 (Stearic acid) | GC-MS | % | 4.4 |
| C18:1n9 (Oleic acid) | GC-MS | % | 59.9 |
| C18:2n6 (Linoleic acid) | GC-MS | % | 6.1 |
| C18:3n3 (alpha-Linolenic acid) | GC-MS | % | 0.4 |

TABLE 4
Vitamin content of dried cell powder of isolated
bacterial strain deposited as VTT-E-193585.

| Parameter | Method | Unit | Value |
|---|---|---|---|
| Vitamin A (RE) | HPLC | IU/100 g | <100 |
| Vitamin E (TE) | HPLC | mg/100 g | 0.33 |
| Vitamin D3 | HPLC | IU/100 g | <10 |
| Vitamin D2 | HPLC | IU/100 g | 21.6 |
| Vitamin C | HPLC | mg/100 g | <1 |
| Thiamine chloride | LC-MS/MS | mg/100 g | 0.9 |
| Hydrochloride | | | |
| Vitamin B1 (Thiamine) | Calculation | mg/100 g | 0.708 |
| Vitamin B2 (Riboflavin) | HPLC | mg/100 g | 6.27 |
| Pyridoxine hydrochloride | HPLC | mg/100 g | 3.39 |
| Vitamin B6 (Pyridoxine) | Calculation | mg/100 g | 2.79 |
| Vitamin B12 | LC-MS/MS | μg/100 g | 224 |
| Choline chloride | LC-MS/MS | mg/100 g | 14.3 |
| Biotin | LC-MS/MS | μg/100 g | 15.6 |
| Folic acid | Microbiological | μg/100 g | 1270 |
| Niacin (Vitamin B3) | Microbiological | mg/100 g | 23.2 |
| Pantothenic acid | Microbiological | mg/100 g | 6.53 |

### Example 3. Cultivation of Isolated Bacterial Strain on Different Nitrogen Sources

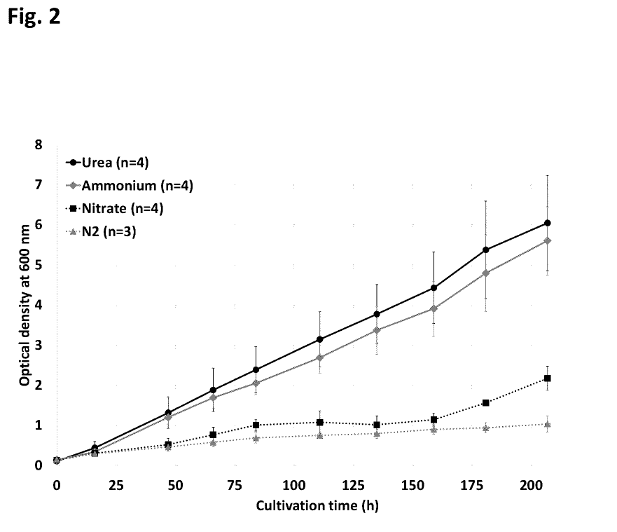

The isolated bacterial strain deposited as VTT-E-193585 was cultivated in a 15-vessel parallel bioreactor system at 200 mL volume (Medicel Explorer, Medicel Oy, Finland). Mixing was performed with Rushton-type impellers rotating at 800 rpm. The temperature in the cultivation was maintained at +30° C. pH was maintained at 6.8 by adding 1 M NaOH. The cultivation medium contained 0.23 g/L KH₂PO₄, 0.29 g/L Na₂HPO₄·2 H₂O, 0.005 g/L NaVO₃·H₂O, 0.2 g/L FeSO₄·7 H₂O, 0.5 g/L MgSO₄·7 H₂O, 0.01 g/L CaSO₄, 0.00015 g/L Na₂MoO₄·2 H₂O, 0.005 g/L MnSO₄, 0.0005 g/L ZnSO₄·7 H₂O, 0.0015 g/L H₃BO₃, 0.001 g/L COSO₄, 0.00005 g/L CUSO₄ and 0.0001 g/L NiSO₄ prepared in tap water. Furthermore, the nitrogen source was varied in the cultivations so that four cultivations contained 18.7 mM NH₄OH, four cultivations contained 9.34 mM urea (OC(NH₂)₂), four cultivations contained 18.7 mM potassium nitrate (KNO₃), and three cultivations were left without nitrogen source in the medium. A mixture containing 22 mL/min hydrogen gas, 3.2 mL/min air and 6.4 mL/min carbon dioxide gas was supplied constantly as the main source of energy and carbon. Thus, with air, all cultivations were also supplied with nitrogen gas. Growth was monitored by taking samples automatically and analysing the cell density as optical density by measuring absorbance at 600 nm (Ultrospec 2100 pro UV/visible spectrophotometer, Biochrom Ltd., England). Growth curves of the cultivations are presented in FIG. 2. Growth on ammonia and urea were comparable. Growth on nitrate or nitrogen gas was clearly slower than on ammonia or urea. Towards the end of the cultivation, the growth on nitrate was better than growth on nitrogen gas as the only source of nitrogen. There was nonetheless growth also in the cultivations in which nitrogen gas was the only source of nitrogen demonstrating that isolated bacterial strain deposited as VTT-E-193585 is capable of nitrogen fixation.

### Example 4. Characterization of Antibiotic Susceptibility

Antibiotic susceptibility of gentamicin, kanamycin, streptomycin, tetracycline, ampicillin, ciprofloxacin, colistin and fosfomycin for the isolated bacterial strain deposited as VTT-E-193585 was analysed according to CLSI M07-A111 standard (Clinical and laboratory standards institute. Methods for dilution antimicrobial susceptibility tests for bacteria that grow aerobically, 11th ed. CLSI standard M07, 2018) with hand-made microdilution plate for ampicillin, ciprofloxacin and colistin, with VetMIC Lact-1 plate (SVA National Veterinary Institute, Uppsala, Sweden) for gentamicin, kanamycin, streptomycin and tetracycline using broth microdilution method and for fosfomycin using agar dilution method in aerobic conditions at +35±2° C. for 48±1 hours using cation-adjusted Mueller Hinton Broth-medium (LabM, LAB114, cations Mg²⁺ and Ca²⁺ added separately). Escherichia coli ATCC 25922 was used as quality control strain and it was incubated in aerobic conditions, at +35±2° C. for 18±2 hours. Results of antibiotic susceptibility of strains are presented in Table 5. The isolation bacterial strain was found to be generally sensitive to antibiotics. For gentamicin, kanamycin, streptomycin and tetracycline minimum inhibitory concentration (MIC) values for VTT-E-193585 were lower or comparable to E. coli ATCC 25922, while for ampicillin, ciprofloxacin, colistin and fosfomycin the MIC values were higher in VTT-E-193585.

TABLE 5
Minimum Inhibitory Concentration (MIC, μg/ml) -values of antibiotics
for VTT-E-193585 strain and Escherichia coli ATCC 25922

| | VTT-E-193585 | E. coli ATCC 25922 |
|---|---|---|
| | 48 h ± 1 h | 18 h ± 2 h |
| Gentamicin | 0.5 | 0.5 |
| Kanamycin | 2 | 4 |
| Streptomycin | 0.5 | 4 |
| Tetracycline | 0.25 | 1 |
| Ampicillin | 16 | 8 |
| Ciprofloxacin | 0.06 | 0.008 |
| Colistin | 4 | 2 |
| Fosfomycin | 32 | 0.5 |

1. An isolated bacterial strain VTT-E-193585 or a derivative thereof, wherein said derivative preferably has retained the ability to grow using hydrogen gas as energy source and carbon dioxide as the only carbon source.

2. A culture comprising the bacterial strain according to claim 1.

3. A process for the production of biomass, said process comprising culturing the bacterial strain of claim 1.

4. The process according to claim 3, comprising culturing the bacterial strain in continuous culture with hydrogen as energy source and an inorganic carbon source, wherein the inorganic carbon source comprises carbon dioxide.

5. A process for the production of biomass, said process comprising culturing a bacterial strain of the genus Xanthobacter in continuous culture with hydrogen as energy source and an inorganic carbon source, wherein the inorganic carbon source comprises carbon dioxide.

6. The process according to claim 4, wherein dissolved oxygen in the culture is maintained between 5% and 10%.

7. The process according to claim 4, wherein ammonium, urea, nitrate and/or nitrogen gas is used as nitrogen source.

8. The process according to claim 4, wherein the culture medium comprises minerals wherein said minerals comprises less than 1 g/L of chloride salts, such as less than 0.25 g/L of chloride salts, e.g. less than 0.1 g/l of chloride salts, preferably no chloride salts.

9. The process according to claim 4, wherein the culture medium does not contain vitamins.

10. The processing according to claim 4, wherein pH in the culture is maintained between 5.5 and 8.0, e.g. between 6.5 and 7.0, such as at 6.8.

11. The process according to claim 4, wherein said culture is grown at a temperature between 25° C. and 40° C., e.g. between 28° C. and 32° C., such as at 30° C.

12. The process according to claim 4, wherein the bacterial strain has a growth rate of 0.04-0.12 h⁻¹.

13. The process according to claim 4, wherein the bacterial strain is VTT-E-193585 or X. tagetidis.

14. The process according to claim 4, wherein the bacterial strain comprises the 16S ribosomal RNA set forth in SEQ ID NO:1 or a 16S ribosomal RNA having up to 20 nucleotide differences with SEQ ID NO:1, e.g. 1 to 10, such as 1 to 5, e.g. one, two or three nucleotide differences with SEQ ID NO:1.

15. The process according to claim 4, wherein the bacterial strain comprises:
i) a gene encoding ribulose-1,5-bisphosphate carboxylase/oxygenase (rubisco) large chain having the sequence set forth in SEQ ID NO:3 or a sequence having more than 93% identity, e.g. more than 95% identity, such as more than 96% identity, e.g. more than 97% identity, such as more than 98% identity, e.g. more than 99% sequence identity to the sequence set forth in SEQ ID NO:3, and/or
ii) a gene encoding a ribulose-1,5-bisphosphate carboxylase/oxygenase (rubisco) small chain having the sequence set forth in SEQ ID NO:5 or a sequence having more than 83% sequence identity, e.g. more than 86%, identity such as more than 90% identity, e.g. more than 95% identity, such as more than 96% identity, e.g. more than 97% identity, such as more than 98% identity, e.g. more than 99% sequence identity to the sequence set forth in SEQ ID NO:5, and/or
iii) a gene encoding a NAD⁺-reducing hydrogenase HoxS subunit alpha having the sequence set forth in SEQ ID NO:7 or a sequence having more than 70% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:7, and/or
iv) a gene encoding a NAD⁺-reducing hydrogenase HoxS subunit beta having the sequence set forth in SEQ ID NO:9 or a sequence having more than 77% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:9, and/or
v) a gene encoding a NAD⁺-reducing hydrogenase HoxS subunit gamma having the sequence set forth in SEQ ID NO:11 or a sequence having more than 70% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:11, and/or
vi) a gene encoding a NAD⁺-reducing hydrogenase HoxS subunit delta having the sequence set forth in SEQ ID NO:13 or a sequence having more than 79% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:13, and/or
vii) a gene encoding a NiFeSe hydrogenase large subunit having the sequence set forth in SEQ ID NO:15 or a sequence having more than 84% sequence identity, e.g. more than 90% identity, such as more than 95% identity, e.g. more than 96% identity, such as more than 97% identity, e.g. more than 98%, such as more than 99% sequence identity to the sequence set forth in SEQ ID NO:15, and/or
viii) a gene encoding a NiFeSe hydrogenase small subunit having the sequence set forth in SEQ ID NO:17 or a sequence having more than 90% identity, such as more than 95% identity, e.g. more than 96% identity, such as more than 97% identity, e.g. more than 98%, such as more than 99% sequence identity to the sequence set forth in SEQ ID NO:17, and/or
ix) a gene encoding an ATP synthase gamma chain atpG_1 having the sequence set forth in SEQ ID NO:19 or a sequence having more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:19, and/or
x) a gene encoding an ATP synthase subunit alpha atpA_1 having the sequence set forth in SEQ ID NO:21 or a sequence having more than 78% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:21, and/or
xi) a gene encoding an ATP synthase subunit b atpF_1 having the sequence set forth in SEQ ID NO:23 or a sequence having more than 62% identity, e.g. more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:23, and/or
xii) a gene encoding an ATP synthase subunit c, sodium ion specific atpE_1 having the sequence set forth in SEQ ID NO:25 or a sequence having more than 90% identity, such as more than 95% identity, e.g. more than 96% identity, such as more than 97% identity, e.g. more than 98%, such as more than 99% sequence identity to the sequence set forth in SEQ ID NO:25, and/or
xiii) a gene encoding an ATP synthase subunit a atpB_1 having the sequence set forth in SEQ ID NO:27 or a sequence having more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:27, and/or
xiv) a gene encoding an ATP synthase epsilon chain atpC_1 having the sequence set forth in SEQ ID NO:29 or a sequence having more than 71% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:29, and/or
xv) a gene encoding an ATP synthase subunit beta atpD_1 having the sequence set forth in SEQ ID NO:31 or a sequence having more than 84% identity, e.g. more than 90% identity, such as more than 95% identity, e.g. more than 96% identity, such as more than 97% identity, e.g. more than 98%, such as more than 99% sequence identity to the sequence set forth in SEQ ID NO:31, and/or xvi) a gene encoding an ATP synthase subunit beta atpD_2 having the sequence set forth in SEQ ID NO:33 or a sequence having more than 97% identity, e.g. more than 98%, such as more than 99% sequence identity to the sequence set forth in SEQ ID NO:33, and/or
xvii) a gene encoding an ATP synthase gamma chain atpG_2 having the sequence set forth in SEQ ID NO:35 or a sequence having more than 86% identity, e.g. more than 90% identity, such as more than 95% identity, e.g. more than 96% identity, such as more than 97% identity, e.g. more than 98%, such as more than 99% sequence identity to the sequence set forth in SEQ ID NO:35, and/or xviii) a gene encoding an ATP synthase subunit alpha atpA_2 having the sequence set forth in SEQ ID NO:37 or a sequence having more than 98%, such as more than 99% sequence identity to the sequence set forth in SEQ ID NO:37, and/or
xix) a gene encoding an ATP synthase subunit delta atpH having the sequence set forth in SEQ ID NO:39 or a sequence having more than 85% identity, e.g. more than 90% identity, such as more than 95% identity, e.g. more than 96% identity, such as more than 97% identity, e.g. more than 98%, such as more than 99% sequence identity to the sequence set forth in SEQ ID NO:39, and/or
xx) a gene encoding an ATP synthase subunit b atpF_2 having the sequence set forth in SEQ ID NO:41 or a sequence having more than 87% identity, e.g. more than 90% identity, such as more than 95% identity, e.g. more than 96% identity, such as more than 97% identity, e.g. more than 98%, such as more than 99% sequence identity to the sequence set forth in SEQ ID NO:41, and/or
xxi) a gene encoding an ATP synthase subunit b′ atpG_3 having the sequence set forth in SEQ ID NO:43 or a sequence having more than 81% identity, e.g. more than 90% identity, such as more than 95% identity, e.g. more than 96% identity, such as more than 97% identity, e.g. more than 98%, such as more than 99% sequence identity to the sequence set forth in SEQ ID NO:43, and/or xxii) a gene encoding ATP synthase subunit c atpE_2 having the sequence set forth in SEQ ID NO:45 or a sequence having more than 98%, such as more than 99% sequence identity to the sequence set forth in SEQ ID NO:45, and/or
xxiii) a gene encoding an ATP synthase subunit a atpB_2 having the sequence set forth in SEQ ID NO:47 or a sequence having more than 92% identity, such as more than 95% identity, e.g. more than 96% identity, such as more than 97% identity, e.g. more than 98%, such as more than 99% sequence identity to the sequence set forth in SEQ ID NO:47, and/or
xiv) a gene encoding an ATP synthase protein I atpI having the sequence set forth in SEQ ID NO:49 or a sequence having more than 60% identity, e.g. more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:49, and/or
xv) a gene encoding a nitrogenase molybdenum-iron protein alpha chain nifD_1 having the sequence set forth in SEQ ID NO:51 or a sequence having more than 60% identity, e.g. more than 70% identity, such as more than 92% identity, such as more than 95% identity, e.g. more than 96% identity, such as more than 97% identity, e.g. more than 98%, such as more than 99% sequence identity to the sequence set forth in SEQ ID NO:51, and/or
xvi) a gene encoding nitrogenase molybdenum-iron protein alpha chain nifD_2 having the sequence set forth in SEQ ID NO:53 or a sequence having more than 60% identity, e.g. more than 98%, such as more than 99% sequence identity to the sequence set forth in SEQ ID NO:53, and/or xvii) a gene encoding a nitrogenase molybdenum-iron protein beta chain nifK_1 having the sequence set forth in SEQ ID NO:55 or a sequence having more than 87% identity, e.g. more than 90% identity, such as more than 95% identity, e.g. more than 96% identity, such as more than 97% identity, e.g. more than 98%, such as more than 99% sequence identity to the sequence set forth in SEQ ID NO:55, and/or
xviii) a gene encoding a nitrogenase molybdenum-iron protein beta chain nifK_2 having the sequence set forth in SEQ ID NO:57 or a sequence having more than 95% identity, e.g. more than 96% identity, such as more than 97% identity, e.g. more than 98%, such as more than 99% sequence identity to the sequence set forth in SEQ ID NO:57, and/or
xxix) a gene encoding a nitrogenase iron protein nifH having the sequence set forth in SEQ ID NO:59 or a sequence having more than 98.5% sequence identity to the sequence set forth in SEQ ID NO:59, wherein the strain preferably comprises:
the genes described in iii), iv), v) and vi),
the genes described in vii) and viii), or
the genes described in iii), iv), v), vi), vii) and viii).

16. The process according to claim 4, comprising the further step of harvesting biomass produced during the culture, optionally comprising a further step of drying the biomass.

17. A process for the production of protein, comprising performing the process according to claim 16 and a further step of isolating protein from said biomass, wherein the process results in a protein fraction and a fraction comprising non-protein components.

18. The process according to claim 16, comprising the further step of producing a food or feed product from said biomass, from said protein fraction or from said fraction comprising non-protein components.

19. A product, such as biomass, protein, or non-protein components obtained or obtainable by the process according to claim 4.

20. A food or feed product obtained or obtainable by the process according to claim 18.


SEQUENCE LISTING (59 sequences)

SEQ ID NO: 1 - DNA, 1482 bp, Unknown
  Novel strain
cttgagagtttgatcctggctcagagcgaacgctggcggcaggcctaacacatgcaagtcgagcgcccagcaatgggagcggcagacgggtgagtaacgcgtggggatgtgcccaatggtacggaataacccagggaaacttggactaataccgtatgagcccttcgggggaaagatttatcgccattggatcaacccgcgtctgattagctagttggtggggtaacggcccaccaaggcgacgatcagtagctggtctgagaggatgatcagccacactgggactgagacacggcccagactcctacgggaggcagcagtggggaatattggacaatgggcgcaagcctgatccagccatgccgcgtgtgtgatgaaggccttagggttgtaaagcactttcgccggtgaagataatgacggtaaccggagaagaagccccggctaacttcgtgccagcagccgcggtaatacgaagggggctagcgttgctcggaatcactgggcgtaaagcgcacgtaggcggatcgttaagtcaggggtgaaatcctggagctcaactccagaactgcccttgatactggcgaccttgagttcgagagaggttggtggaactgcgagtgtagaggtgaaattcgtagatattcgcaagaacaccagtggcgaaggcggccaactggctcgatactgacgctgaggtgcgaaagcgtggggagcaaacaggattagataccctggtagtccacgccgtaaacgatggatgctagccgttgggcagcttgctgttcagtggcgcagctaacgcattaagcatcccgcctggggagtacggtcgcaagattaaaactcaaaggaattgacgggggcccgcacaagcggtggagcatgtggtttaattcgaagcaacgcgcagaaccttaccagcctttgacatggcaggacgatttccagagatggatctcttccagcaatggacctgcacacaggtgctgcatggctgtcgtcagctcgtgtcgtgagatgttgggttaagtcccgcaacgagcgcaaccctcgcctctagttgccagcattcagttgggcactctagagggactgccggtgataagccgagaggaaggtggggatgacgtcaagtcctcatggcccttacgggctgggctacacacgtgctacaatggtggtgacagtgggatgcgaaagggcgacctctagcaaatctccaaaagccatctcagttcggattgtactctgcaactcgagtgcatgaagttggaatcgctagtaatcgtggatcagcatgccacggtgaatacgttcccgggccttgtacacaccgcccgtcacaccatgggagttggctttacccgaaggcgctgcgctaacccgcaagggaggcaggcgaccacggtagggtcagcgactggggtgaagtcgtaacaaggtagccgtaggggaacctgcggctggatcacctccttt

SEQ ID NO: 2 - DNA, 1467 bp, Unknown
  novel strain
atgggtgccgaagcaaccgtcgggcagatcacggacgccaagaagagatacgccgccggcgtgctgaagtacgcccagatgggctactggaacggcgactacgttcccaaggacaccgacctcctggcggtgttccgcatcaccccccaggcgggcgtggacccggtggaagccgccgcggcggtcgccggcgaaagctccaccgctacctggaccgtggtgtggaccgaccggctcaccgccgccgacgtctaccgcgccaaggcctacaaggtggagccggtgccgggccaggaaggccagtatttctgctacatcgcctatgatctcgatttgttcgaggaaggctccatcgccaacctcacggcgtcgatcatcggcaacgtcttctccttcaagccgctgaaggcggcgcggctggaggacatgcggcttcccgtcgcctatgtgaagaccttccgcggcccgcccaccggcatcgtggtcgagcgcgagcgcctggacaagttcggccgcccccttctgggcgccaccaccaagccgaagcttggcctctcgggcaagaattacggccgcgtggtctatgaggccctcaagggcggcctcgacttcgtgaaggacgacgagaacatcaactcgcagcccttcatgcactggcgcgatcgcttcctctattgcatggaggccgtcaacaaggcccaggccgagaccggcgaggtgaaggggcactatctcaacatcaccgccgggaccatggaggagatgtaccgccgcgccgagttcgccaaggaactgggctccgtggtggtgatggtggatctcatcatcggctggaccgccatccagtccatgtccaactggtgccgcgagaacgacatgatcctgcacatgcaccgtgcgggccatggcacctacacgcgccagaagagccacggcgtctccttccgcgtcatcgccaagtggctgcggctcgccggcgtcgaccacctgcacaccggcaccgccgtgggcaagctggaaggcgaccccatgaccgtgcagggcttctacaatgtctgccgcgagacgacgacgcagcaggacctcacccgcggcctgttcttcgagcaggactggggcggcatccgcaaggtgatgccggtggcctccggcggcatccatgcgggccagatgcaccagctcatcgacctgttcggcgaggacgtggtgctccagttcggcggcggcaccatcggccacccggacggcatccaggccggcgccaccgccaaccgcgtggcgctggaaaccatgatcctcgcccgcaacgagggccgcgacatcaggaacgagggcccggaaatcctggtggaagccgccaaatggtgccgtccgctgcgcgcggcgctcgatacctggggcgaggtgaccttcaactacgcctccaccgacacgtccgattacgtgcccaccgcgtccgtcgcctga

SEQ ID NO: 3 - protein, 488 aa, Unknown
  novel strain
MGAEATVGQITDAKKRYAAGVLKYAQMGYWNGDYVPKDTDLLAVFRITPQAGVDPVEAAAAVAGESSTATWTVVWTDRLTAADVYRAKAYKVEPVPGQEGQYFCYIAYDLDLFEEGSIANLTASIIGNVFSFKPLKAARLEDMRLPVAYVKTFRGPPTGIVVERERLDKFGRPLLGATTKPKLGLSGKNYGRVVYEALKGGLDFVKDDENINSQPFMHWRDRFLYCMEAVNKAQAETGEVKGHYLNITAGTMEEMYRRAEFAKELGSVVVMVDLIIGWTAIQSMSNWCRENDMILHMHRAGHGTYTRQKSHGVSFRVIAKWLRLAGVDHLHTGTAVGKLEGDPMTVQGFYNVCRETTTQQDLTRGLFFEQDWGGIRKVMPVASGGIHAGQMHQLIDLFGEDVVLQFGGGTIGHPDGIQAGATANRVALETMILARNEGRDIRNEGPEILVEAAKWCRPLRAALDTWGEVTFNYASTDTSDYVPTASVA

SEQ ID NO: 4 - DNA, 405 bp, Unknown
  novel strain
atgcgcatcacccaaggctccttctccttcctgccggacctcaccgacacgcagatcaaggcccaggtgcaatattgcctggaccagggctgggcggtctcggtggagcacaccgacgatccccacccgcgcaacacctattgggagatgtggggcccgcccatgttcgatctgcgcgacgcggccggcgtcttcggcgagatcgaagcctgccgggccgccaatcccgagcattatgtgcgggtgaacgccttcgattccagccgcggatgggagacgatccgcctgtccttcatcgttcagcggcccaccgtggaagagggcttccgcctcgaccgcaccgaaggcaagggccgcaaccagagctacgccatgcgctaccgggcgcagttcgcgccgcgctga

SEQ ID NO: 5 - protein, 134 aa, Unknown
  novel strain
MRITQGSFSFLPDLTDTQIKAQVQYCLDQGWAVSVEHTDDPHPRNTYWEMWGPPMFDLRDAAGVFGEIEACRAANPEHYVRVNAFDSSRGWETIRLSFIVQRPTVEEGFRLDRTEGKGRNQSYAMRYRAQFAPR

SEQ ID NO: 6 - DNA, 1896 bp, Unknown
  novel strain
atgatgccatctgagccgcacggcgcgggcatgccgcccccacgggaagcggccgcggttcccaccccccaggaggtgagcgcggtggtggccgaggtggtcgcggatgccgtggcatcggtgggcggcgcacgcacccggctcatggacatcgtccagctggcccagcagcgtctcggccatctctccgaagagaccatggcggccattgccgcgcggctcgccattccgccggtggaagtggcggacatggtgtccttctacgccttcctgaaccgcgcgcccaagggccgctaccacatccgcctgtcgcgcagccccatctcgctgatgaagggcgccgaggcggtggctgccgccttctgccagatcctcggcatcgccatgggcgagacctcgcaggatggcgacttcaccctggaatggaccaacgacatcggcatggccgaccaggagccggccgccctcgtcaacggcacggtgatgacgcagctcgcgcccggcgatgcggccatcatcgtcggccggctgcgggcccatcacgcgcccaatgccctgccgctgttccctggagccggcgtggccggctccggcctgccccatgcccggatccgccccagcctggtgatgccgggacagcttctgttccgcgaggaccacacgacgccgggcgccggcatcaaggcggcactcgccctcaccccggacgaagtggtgcagaaggtctccgccgcgcgcctgcgcgggcggggtggcgccggctttcccaccggtctcaaatggaagctctgccgccagtcgcccgccaccacccgccatgtgatctgcaatgcggacgagggcgagcccggcaccttcaaggatcgcgtgctgctcacgcaggcgccgcacctcatgttcgacggcatgaccatcgccggctacgccttgggggcgcgggagggcgtggtctatctgcgcggcgagtacgcctatctgtgggagcctctgcatgcggtcctgcgcgagcgctatgggctcgggctcgccggcgcgaacatcctgggacacgcgggcttcgacttcgacatccgcatccagctgggcgccggcgcctatatctgcggcgaggaatccgcgctggtggaatcgctggaaggcaagcgcggctcgccccgcgaccgcccccccttccccaccgtgcgcggccatctccagcagcccaccgccgtggacaatgtggagaccttcgcctgcgccgcccgcatcctggaggatggcgtggaggcgttcgcgggcatcggcacgcccgaatccgccggcacgaagctcctctcggtgtcgggcgattgcccgcgccccggcgtgtatgaggtgcccttcggcctcacggtgaacgcgctgctcgaccttgtcggcgcgccggacgccgccttcgtgcagatgggtgggccgtccggccaatgcgtggcgccgaaggattacggccgccgcatcgccttcgaggacctgcccaccggcggctcggtgatggtgttcggcccggggcgcgacgtgctcgccatggtgcgcgagttcgcggatttcttcgccggcgaatcctgcggctggtgcacgccctgccgggtgggcaccaccttgctcaaggaagagctggacaagctcctcgccaaccgcgccaccctcgccgacatccgcgcgctggagaccctggccacgaccgtctcccgcaccagccgctgcggcctcggccagacggcgcccaaccccatcctttccaccatgcgcaacctgccggaagcctatgaggcgaggctgaggcccgaagacttcctgccctgggcctcgctcgacgaggcgctgaagcccgccatcgtcatccagggccgcgcgcccgtgccggaggaagaggcatga

SEQ ID NO: 7 - protein, 631 aa, Unknown
  novel strain
MMPSEPHGAGMPPPREAAAVPTPQEVSAVVAEVVADAVASVGGARTRLMDIVQLAQQRLGHLSEETMAAIAARLAIPPVEVADMVSFYAFLNRAPKGRYHIRLSRSPISLMKGAEAVAAAFCQILGIAMGETSQDGDFTLEWTNDIGMADQEPAALVNGTVMTQLAPGDAAIIVGRLRAHHAPNALPLFPGAGVAGSGLPHARIRPSLVMPGQLLFREDHTTPGAGIKAALALTPDEVVQKVSAARLRGRGGAGFPTGLKWKLCRQSPATTRHVICNADEGEPGTFKDRVLLTQAPHLMFDGMTIAGYALGAREGVVYLRGEYAYLWEPLHAVLRERYGLGLAGANILGHAGFDFDIRIQLGAGAYICGEESALVESLEGKRGSPRDRPPFPTVRGHLQQPTAVDNVETFACAARILEDGVEAFAGIGTPESAGTKLLSVSGDCPRPGVYEVPFGLTVNALLDLVGAPDAAFVQMGGPSGQCVAPKDYGRRIAFEDLPTGGSVMVFGPGRDVLAMVREFADFFAGESCGWCTPCRVGTTLLKEELDKLLANRATLADIRALETLATTVSRTSRCGLGQTAPNPILSTMRNLPEAYEARLRPEDFLPWASLDEALKPAIVIQGRAPVPEEEA

SEQ ID NO: 8 - DNA, 1491 bp, Unknown
  novel strain
atgagccggggatcccccgatgccgggaaagaccgcaccatgagcgccaccgacggcaccaccgccccccgcaagatcgtcatcgatccggtgacccgcgtggagggccacggcaaggtcaccatccgcctggatgaagccggcgcggtggaggatgcgcgtttccacatcgtggagttccgcggcttcgagcggttcatccagggccggatgtactgggaagtgccccttatcatccagcggctgtgcggcatctgcccggtgagccaccatctggcggcggcgaaagccatggaccaggtggcgggcgtggaccgcgtaccgcccaccgccgagaaactgcgccggctgatgcattatgggcaggtgctgcaatccaacgctttgcacatcttccacctcgcctcgcccgacctcctgttcggcttcgacgcgccggccgagcagcgcaacatcatcgccgtgctccagcgttatccggagatcggcaaatgggcgatcttcatcaggaagttcggccaggaggtcatcaaggccaccggcgggcgcaagatccatcccaccagcgccattcccggcggggtcaaccagaacctcgccgtggaggaccgcgacgccctgcgcgccaaggtgggcgagatcatcagctggtgcatggcggcgctggaccatcacaaggcctatgtggcggaaaaccgggcgctgcatgacagcttcgccgccttcccctccgccttcatgagcctcgtggggccggatggcggcatggacctttatgacggcaccctgcgggtgatcgatgccgagggcgcccccctcatcgaaggcgcgccgcccgcctcctaccgcgaccacctcatcgaggaggtgcggccctggagctatctgaaattcccccatctgcgcgccttcggccgcgacgatggctggtatcgggtcggccccctcgcccaggtcaattgcgccgcgtccatcgacacgccccgcgccgaggcggcccggcgggacttcatggccgagggcggcggcaagccggtgcatgccaccctcgcttatcactgggcgcggctcatcgtgctggtccattgcgcggagaagatcgaacagctgctgttcgacgacgacctgcaaggctgcgatctgcgtgcggagggcacccggcgcggggaaggcgtcgcctggatcgaggcgccgcgcggcaccctcatccaccattacgaggtggacgagaacgaccaggtgcgccgcgccaacctcatcgtctccaccacccacaataacgaggccatgaaccgcgccgtgcggcaggtggcgaagacggacctttccggtcgcgagatcaccgaagggctgctgaaccatatcgaggtggccatccgcgccttcgacccctgcctgtcctgcgccacccatgcgctgggccagatgccgctgatcgtgacgcttgaagatgcctccggcgcagagatcgcccgcggagtgaaggaatga

SEQ ID NO: 9 - protein, 496 aa, Unknown
  novel strain
MSRGSPDAGKDRTMSATDGTTAPRKIVIDPVTRVEGHGKVTIRLDEAGAVEDARFHIVEFRGFERFIQGRMYWEVPLIIQRLCGICPVSHHLAAAKAMDQVAGVDRVPPTAEKLRRLMHYGQVLQSNALHIFHLASPDLLFGFDAPAEQRNIIAVLQRYPEIGKWAIFIRKFGQEVIKATGGRKIHPTSAIPGGVNQNLAVEDRDALRAKVGEIISWCMAALDHHKAYVAENRALHDSFAAFPSAFMSLVGPDGGMDLYDGTLRVIDAEGAPLIEGAPPASYRDHLIEEVRPWSYLKFPHLRAFGRDDGWYRVGPLAQVNCAASIDTPRAEAARRDFMAEGGGKPVHATLAYHWARLIVLVHCAEKIEQLLFDDDLQGCDLRAEGTRRGEGVAWIEAPRGTLIHHYEVDENDQVRRANLIVSTTHNNEAMNRAVRQVAKTDLSGREITEGLLNHIEVAIRAFDPCLSCATHALGQMPLIVTLEDASGAEIARGVKE

SEQ ID NO: 10 - DNA, 720 bp, Unknown
  novel strain
atgagcgagacccccttcacctttaccgtggacggcatcgcggtcccggccacccccggccagagcgtcatcgaggcgtgcgatgcggcgggcatctatatcccgcgcctgtgccaccacccggacctgccgccggcgggccattgccgggtgtgcacctgcatcatcgacgggcggccggccagcgcctgcaccatgcccgccgccaggggcatggtggtggagaacgagacgcccgctttgctggcggagcggcgcacgctgatcgagatgctgttcgcggaaggcaaccatttctgccagttctgcgaggcgagcggcgattgcgaattgcaggcgctgggctacctgttcggcatggtggccccgcccttcccccatctgtggccgaagcggccggtggatgccagccatccggatatctatatcgaccacaatcgctgcatcctgtgctcgcgctgcgtgcgcgcctcgcgcaccctggacggcaagtccgtgttcggcttcgaggggcgcggcatcgagatgcatctggcggtgaccggcgggcacctggacgacagcgccatcgccgccgccgacagggcggttgagatgtgcccggtgggctgcatcgtcctcaagcgcaccggctaccgcacgccctatggccggcggcgctacgacgccgcgcccatcggctccgacatcaccgcccggcgcggcggcgcgaaggactga

SEQ ID NO: 11 - protein, 239 aa, Unknown
  novel strain
MSETPFTFTVDGIAVPATPGQSVIEACDAAGIYIPRLCHHPDLPPAGHCRVCTCIIDGRPASACTMPAARGMVVENETPALLAERRTLIEMLFAEGNHFCQFCEASGDCELQALGYLFGMVAPPFPHLWPKRPVDASHPDIYIDHNRCILCSRCVRASRTLDGKSVFGFEGRGIEMHLAVTGGHLDDSAIAAADRAVEMCPVGCIVLKRTGYRTPYGRRRYDAAPIGSDITARRGGAKD

SEQ ID NO: 12 - DNA, 543 bp, Unknown
  novel strain
atggccaagcccaaactcgccacctgcgcgctggccggctgcttcggctgccacatgtccttcctggacatggacgagcgcatcgtcgagctcatcgacctggtggacctcgacgtctcgcccctcgacgacaagaaaaacttcaccggcatggtggaaatcggcctggtggaaggcggctgcgccgacgagcgccatgtgaaggtgctgcgcgagttccgcgagaaatcccgcatcctggtggcggtgggcgcctgcgccatcaccggcggcatcccggcattgcgcaacctcgccggcctcgacgaatgcctgagggaagcctacctcaccggccccacggtggaaggcggcgggctcattcccaacgacccggagctgccgctgctgctggacaaggtctatccggtgcaggacttcgtgaagatcgaccatttcctgcccggctgcccgccctcggccgacgccatctgggcggctctgaaggcgctgctgaccggcaccgagccgcatctgccctacccgcttttcaagtacgaatga

SEQ ID NO: 13 - protein, 180 aa, Unknown
  novel strain
MAKPKLATCALAGCFGCHMSFLDMDERIVELIDLVDLDVSPLDDKKNFTGMVEIGLVEGGCADERHVKVLREFREKSRILVAVGACAITGGIPALRNLAGLDECLREAYLTGPTVEGGGLIPNDPELPLLLDKVYPVQDFVKIDHFLPGCPPSADAIWAALKALLTGTEPHLPYPLFKYE

SEQ ID NO: 14 - DNA, 1013 bp, Unknown
  novel strain
tccagacccgggcaacattgctccatgtgctgggcaccctggccggccgctggccccataccctcgcgctccagcccggcggggtgacccgaagcgccgaccagcacgaccgcatgcgcctgctcgcgacgctgaaggcggtgcgggcggcgctggaagagaccttgttcggcgcgcctttggaagaggtggcggccctggacggcgccgccgccgtggaggcctggcgcgccaacggcccggaaggggatttccgcctgttcctggagatcgccgccgacctggagctggaccggctcggccgcgcgcacgaccgctttctctccttcggcgcctacgcccaggacgaggggcgcctttatggcgccggcaccttcgaggccgggacggcgggagggctcgatcccaacgccatcaccgaggaccacgccttcgcccgcatggaggaccgcgcggcgccccatgcgccctttgacggctccaccttccccgatgccgacgacaccgagggctacacctggtgcaaggcgccgcgccttgccggcctgcccttcgagaccggcgccttcgcccggcaggtggtggcgggccatccgctcgcccgggacctcgtgacgcgggaaggcggcactgtgcgcagccgcgtggtcggccggctgctggaaaccgcgcgcaccctgatcgccatggagggctgggtgaaggaactgcggcccgaagggccctggtgcgcccagggccacctgccccaggaaggccgcgccttcggcctcaccgaggcggcgcgcggggcgctcggccactggatggtggtggagaagggccgcattgcccgctaccagatcatcgcccccaccacctggaacttctccccccgcgacggcgcgggcctgcccggcccgctggagacggccctggtgggcgcgcccgtgcggcagggagagacgacgcccgtgagcgtgcagcacatcgtgcgctccttcgacccgtgcatggtctgcactgtgcattga

SEQ ID NO: 15 - protein, 485 aa, Unknown
  novel strain
MSAETRRLVVGPFNRVEGDLEVRLDVQDGRVQQAFVSSPLFRGFERILEGRDPRDALVIAPRICGICSVSQSHAAALALAGLQGIAPTHDGRIATNLIVAAENVADHLTHFHVFFMPDFARAVYEDRPWFAQAARRFKANQGVSVRRALQTRATLLHVLGTLAGRWPHTLALQPGGVTRSADQHDRMRLLATLKAVRAALEETLFGAPLEEVAALDGAAAVEAWRANGPEGDFRLFLEIAADLELDRLGRAHDRFLSFGAYAQDEGRLYGAGTFEAGTAGGLDPNAITEDHAFARMEDRAAPHAPFDGSTFPDADDTEGYTWCKAPRLAGLPFETGAFARQVVAGHPLARDLVTREGGTVRSRVVGRLLETARTLIAMEGWVKELRPEGPWCAQGHLPQEGRAFGLTEAARGALGHWMVVEKGRIARYQIIAPTTWNFSPRDGAGLPGPLETALVGAPVRQGETTPVSVQHIVRSFDPCMVCTVH

SEQ ID NO: 16 - DNA, 858 bp, Unknown
  novel strain
acgggggaggaagcccgcgccatcttcgacgccatccttgccggcgttatcgtcctcgacgccctgtgcgtggaaggcgcgctgctgcgcgggccgaacggcaccgggcgcttccatgtgctggcgggcacggacacccccaccatcgactgggcgcggcagctcgccggcatggcgcgccacgtggtggcggtgggcacctgcgccgcctatgggggcgtgacggcggcgggcatcaaccccaccgatgcctgcggcctccagttcgacggacgccggaagggtggggcgctgggggcggacttccgctcccgctcggggcttccggtcatcaatgtggccggctgccccacccatcccaactgggtgacggaaaccctgatgctgctcgcctgcggcctgctgggcgaggccgacctcgacgtctatggccgcccgcgcttctatgcggacctgctggtgcatcacggctgcccgcgcaacgaatactatgaatacaaggcgagcgccgagaagatgagcgacctcggctgcatgatggagcatctgggctgcctcggcacccaggcccacgccgactgcaacacgcgcctttggaatggcgagggctcgtgcacccgcggcggctatgcctgcatcaactgcacggcgccggaattcgaggagccgggccacgccttcctggagacgcccaagatcggcggcatccccatcggcctgcccaccgacatgcccaaggcctggttcatcgccttgtcctccctcgccaaggcggcgacgccggagcggctgcgcaagaacgcggtgtccgaccatgtggtcacgccgcccgccgtcaaggacatcaagcggcgatga

SEQ ID NO: 17 - protein, 335 aa, Unknown
  novel strain
MSTPFSVLWLQSGGCGGCTMSLLCAEAPDLATTLDAAGIGFLWHPALSEETGEEARAIFDAILAGVIVLDALCVEGALLRGPNGTGRFHVLAGTDTPTIDWARQLAGMARHVVAVGTCAAYGGVTAAGINPTDACGLQFDGRRKGGALGADFRSRSGLPVINVAGCPTHPNWVTETLMLLACGLLGEADLDVYGRPRFYADLLVHHGCPRNEYYEYKASAEKMSDLGCMMEHLGCLGTQAHADCNTRLWNGEGSCTRGGYACINCTAPEFEEPGHAFLETPKIGGIPIGLPTDMPKAWFIALSSLAKAATPERLRKNAVSDHVVTPPAVKDIKRR

SEQ ID NO: 18 - DNA, 870 bp, Unknown
  novel strain
gtgaccgagcgcctgtccgacgtcaacgcccgcatcgcctcggtgcggcagctctcatcggtcatcacggccatgcggggcattgcggcggcgcgggcgcgggaggcgcggggtcggctcgacggcatccgcgcctatgcgcagaccatcgccgaggccatcggccatgtgctcgccgtgctgcccgaggaggcccgcgcccggtcctccgggcaccggcatcggggccatgcggtcatcgccctgtgcgcggagcagggctttgccggcgtcttcaacgagcgggtgctggacgaggccgcccggctgctgaccggcggggcggggccggccgagctgctgctggtgggcgaccggggcctgatggtggcccgcgagcgggggctcgatgtctcctggtcggtgcccatggtggcccatgcgggccaggcctcggcgctggcggaccgcatcagcgaggagctctaccggcggatcgatgcgggacgggtgacgcgggtgtcggtggtgcacgccgagcccgccgcgtccgccgccatcgagacggtggtgaaagtgctggtgccgttcgacttcgcccgcttccccctggcgcgggtggcatccgccccgctcatgaccatgccgccgccgcggctgctggcccagctgtcggaggaatatgtgttcgccgagctgtgcgaggcgctcaccttgtccttcgcggcggagaacgaggcccgcatgcgggccatgatcgccgcccgcgccaatgtggccgataccctggagggcctcgtcggccgcgcccggcagatgcgccaggaggagatcaccaacgagatcatcgagctggaaggcggcgccggcagcgcccggcatgcggattga

SEQ ID NO: 19 - protein, 289 aa, Unknown
  novel strain
MTERLSDVNARIASVRQLSSVITAMRGIAAARAREARGRLDGIRAYAQTIAEAIGHVLAVLPEEARARSSGHRHRGHAVIALCAEQGFAGVFNERVLDEAARLLTGGAGPAELLLVGDRGLMVARERGLDVSWSVPMVAHAGQASALADRISEELYRRIDAGRVTRVSVVHAEPAASAAIETVVKVLVPFDFARFPLARVASAPLMTMPPPRLLAQLSEEYVFAELCEALTLSFAAENEARMRAMIAARANVADTLEGLVGRARQMRQEEITNEIIELEGGAGSARHAD

SEQ ID NO: 20 - DNA, 1536 bp, Unknown
  novel strain
atgagcacgggcgcgcaagcgagcgaggattggctcacccggagccgggcggccctggccgggacgcgcctttcccagcaatcccaatcggtgggccgggtggaggagatggccgacggcatcgcccgcgtctccggcctgccggatgtgcggctcgacgagcttctcaccttcgagggcggccagaccggctatgccctcaccctcgatcgcaccgagatcgccgtggtgctgctggatgacgcctccggcgtggaggcgggcgcccgggtgttcggcaccggcgaggtggtgaaggtgccggtggggccggggctgctgggccgcatcgtcgaccccctcggccggcccatggaccgctccgagccggtggtggcgcaggcgcaccatcccatcgagcggccggcgccggccatcatcgcccgcgacctggtctcgcagccggttcagaccggcacgctggtggtggatgcgctgttctccctcggccggggccagcgcgagctcatcatcggcgaccgggctaccggcaagaccgccatcgcggtggacaccatcatcagccagaagcattcggacatcgtgtgcatctacgtggcggtgggccagcgcgccgccgccgtggagcgggtggtggaggcggtgcgcgcccacggggcgatcgagcgctgcatcttcgtggtcgcctcggccgccgcctcgccagggctgcaatggatcgcgccgttcgccggcatgaccatggcggaatatttccgcgacaacggccagcatgcgctcatcatcatcgatgatctcaccaagcatgcggccacccatcgcgagctggcgctgctcacccacgagccgccgggccgcgaggcctatcccggcgacatcttctatgtgcacgcccgccttctggagcgggccgccaagctctccgccgagctgggcggtggctcgctcacggccctgcccatcgcggagacggacgcgggaaacctctccgcctatatccccaccaacctcatctccatcaccgatgggcagatcgtgctggattcgcggctgttcgcggccaaccagcgcccggcggtggatgtgggcctctccgtgagccgggtgggcggcaaggcgcagcatcccgcgcttcgggccgtgtccgggcgcatccggctcgattattcccagttcctggagctggaaatgttcacccgcttcggcggcatcaccgatacccgcgtgaaggcgcagatcacccggggcgagcgcatccgcgcgctgctcacccagccgcgcttttccaccctgcgccttcaggacgaggtggcgctgctggccgcgctggcggagggggtgttcgacactttggccccggggctgatgggcgccgtgcgtgcccgcattccggcccagctggatgcgcaggtgaaggacgtggcctcggccctcgccgagggcaaggtgctggaggagggcttgcacgcccgtctcgtggcggccgtgcgggccgtcgcggcggacgtggccgcgaccgcgaaggccgggccgtga

SEQ ID NO: 21 - protein, 511 aa, Unknown
  novel strain
MSTGAQASEDWLTRSRAALAGTRLSQQSQSVGRVEEMADGIARVSGLPDVRLDELLTFEGGQTGYALTLDRTEIAVVLLDDASGVEAGARVFGTGEVVKVPVGPGLLGRIVDPLGRPMDRSEPVVAQAHHPIERPAPAIIARDLVSQPVQTGTLVVDALFSLGRGQRELIIGDRATGKTAIAVDTIISQKHSDIVCIYVAVGQRAAAVERVVEAVRAHGAIERCIFVVASAAASPGLQWIAPFAGMTMAEYFRDNGQHALIIIDDLTKHAATHRELALLTHEPPGREAYPGDIFYVHARLLERAAKLSAELGGGSLTALPIAETDAGNLSAYIPTNLISITDGQIVLDSRLFAANQRPAVDVGLSVSRVGGKAQHPALRAVSGRIRLDYSQFLELEMFTRFGGITDTRVKAQITRGERIRALLTQPRFSTLRLQDEVALLAALAEGVFDTLAPGLMGAVRARIPAQLDAQVKDVASALAEGKVLEEGLHARLVAAVRAVAADVAATAKAGP

SEQ ID NO: 22 - DNA, 741 bp, Unknown
  novel strain
atgcagatcgactggtggacgctgggcctgcagacggtcaacgtcctcgttctcatctggctcctgagccgcttcctgttcaagccggtggcgcaggtcatcgcgcagcgccgtgccgagatcgagaagctggtggaggatgcgcgcgccgccaaggccgccgccgaggccgagcgggacacggcgaaggcggaggaggcgcgccttgccgccgagcgcggcgcccgcatggcggcggtcgccaaggaggcggaggcgcagaaggcggcattgctggccgccgccaagaccgaggccgaggccctgcacgcggccgcggaagcggccatcgtccgggcgcgggcgagcgaggaggaagccgccgccgaccgcgccagccgccttgccgtggacatcgccgccaagctgctggaccggctgcccgacgacgcccgggtcgcgggcttcatcgatggcctcgccgaggggcttgaagccctgcccgaggcgagccgggcggtgatcggcgtcgacggcgcgccagtgcgcgtgacggccgcgcgcgcccttatgccggcggaggaggaggcctgccgcacgcggctctcccaggcgctgggccgtccggtgacgctggccgtgaccatcgaccccgccctcatcgccggcctggagatggagacgccccacgcggtggtgcgcaattccttcaaggccgatctcgaccgcgtcaccgcggcgctcacccatcatgggacctga

SEQ ID NO: 23 - protein, 246 aa, Unknown
  novel strain
MQIDWWTLGLQTVNVLVLIWLLSRFLFKPVAQVIAQRRAEIEKLVEDARAAKAAAEAERDTAKAEEARLAAERGARMAAVAKEAEAQKAALLAAAKTEAEALHAAAEAAIVRARASEEEAAADRASRLAVDIAAKLLDRLPDDARVAGFIDGLAEGLEALPEASRAVIGVDGAPVRVTAARALMPAEEEACRTRLSQALGRPVTLAVTIDPALIAGLEMETPHAVVRNSFKADLDRVTAALTHHGT

SEQ ID NO: 24 - DNA, 243 bp, Unknown
  novel strain
atgactgtcgagatggtcagcatcttcgcggcggcgctcgccgtctccttcggcgccatcgggccggccctgggcgagggccgggcggtggccgcggccatggacgccatcgcccgccagccggaggcggccggaaccttgtcgcgcacgctcttcgtcggcctcgccatgatcgagaccatggcgatctactgcctggtgatcgcgctcctggtgctcttcgccaatccgttcgtgaagtga

SEQ ID NO: 25 - protein, 80 aa, Unknown
  novel strain
MTVEMVSIFAAALAVSFGAIGPALGEGRAVAAAMDAIARQPEAAGTLSRTLFVGLAMIETMAIYCLVIALLVLFANPFVK

SEQ ID NO: 26 - DNA, 699 bp, Unknown
  novel strain
atgggctcgccgctgatcctcgaacccctgttccatatcgggcccgtgcccatcaccgcgccggtggtggtcacctggctcatcatggccgccttcattgggctggcgcggctcatcacccggaagctttccaccgatcccacccggacccaggcggcggtggaaacggtgctgaccgccatcgattcccagatcgccgacaccatgcaggccgatcccgcgccttatcgcgcgctcatcggcaccatcttcctttatgtgctggtggccaactggtcctcgctcatcccgggcatcgagccgcccacggcgcatatcgagaccgatgcggcgctcgctttcatcgtgttcgccgccaccatcgggttcgggttgaagacaaggggtgtgaagggctatctcgccaccttcgccgaaccctcctgggtgatgatcccgctcaatgtggtggagcagatcacccggaccttctcgctcatcgtgcgcctgttcggcaacatcatgagcggggtgttcgtggtcggcatcatcctgtccctcgccgggctgctggtgcccatccccctcatggcgctcgatctcctgaccggcgccgtgcaggcctacatcttcgcggtgctggcctgcgtgttcatcggcgcggccattggcgaggcgccggcaaagccccaatcgaaggagccagggaaaacatcatga

SEQ ID NO: 27 - protein, 232 aa, Unknown
  novel strain
MGSPLILEPLFHIGPVPITAPVVVTWLIMAAFIGLARLITRKLSTDPTRTQAAVETVLTAIDSQIADTMQADPAPYRALIGTIFLYVLVANWSSLIPGIEPPTAHIETDAALAFIVFAATIGFGLKTRGVKGYLATFAEPSWVMIPLNVVEQITRTFSLIVRLFGNIMSGVFVVGIILSLAGLLVPIPLMALDLLTGAVQAYIFAVLACVFIGAAIGEAPAKPQSKEPGKTS

SEQ ID NO: 28 - DNA, 456 bp, Unknown
  novel strain
gtgagcgcgccgctgcacctcaccatcaccacgccggccgccgttctggtggaccgtgccgacatcgtggccctgcgtgccgaggacgagagcggcagcttcggcatcctgcccggccatgcggatttcctgaccgttctggaggcctgcgtggtgcgcttcaaggatggggccgacggcgtgcattattgtgctctcagtggtggcgtgctgtcggtcgaggagggccggcgcatcgccatcgcctgccgtcagggcacggtgagcgacgacctggtcgccctggaaggggcggtggacgccatgcgttcggcggagagcgatgccgacaagcgggcccgggtggagcagatgcgccttcatgcccacgccgtgcgccagctcctgcactatctgcggcccggccgggccggcggcgtggcgccggccgccgcgccggaggaggggccgtcatga

SEQ ID NO: 29 - protein, 151 aa, Unknown
  novel strain
MSAPLHLTITTPAAVLVDRADIVALRAEDESGSFGILPGHADFLTVLEACVVRFKDGADGVHYCALSGGVLSVEEGRRIAIACRQGTVSDDLVALEGAVDAMRSAESDADKRARVEQMRLHAHAVRQLLHYLRPGRAGGVAPAAAPEEGPS

SEQ ID NO: 30 - DNA, 1494 bp, Unknown
  novel strain
atggcagcggcagatgaggaggcgcaatcggccgccggccccgcctcgggccgggtggtggccgtgcgcggcgcggtgatcgacatcgcctttgcccagcctccgctgccgccgctggacgacgcccttctcatcaccgacggccggggcggcacggtgctggtggaggtgcagagccatatggatcggcacacggtgcgcgccatcgcccttcaggccaccaccggcctcagccgggggctggaggcggcgcgggtgggcgggccggtgaaggtgccggtgggagaccatgtgctcggccgcctcctggatgtcaccggcgccatcggcgacaagggcgggccgctgccggccgacgtgcccacgcggccgatccaccacgcgccgccatccttcgccgcgcagggcggcacgtccgatctgtttcgcaccggcatcaaggtcatcgacctcctggcgcccctcgcccagggcggcaaggcggccatgttcggcggggccggcgtgggcaagaccgtgctggtgatggagctgatccacgccatggtggcgagctacaagggcatctcggtgtttgccggcgtgggggagcgctcccgcgagggccacgagatgctgctggacatgaccgattccggcgtgctcgaccgcaccgttctggtctatggccagatgaacgagccccccggggcccgctggcgggtgcccatgacggcgctgaccatcgccgaatatttccgcgacgagaagcaccagaacgtcctgctgctgatggacaacatcttccgcttcgtccaggcgggggcggaggtctccggccttttgggccgtccgccctcccgggtgggataccagccgacgctggcgagcgaggtggcggcgctccaggaacgcatcacctccgtgggcgaggcctcggtgaccgccatcgaggcggtctacgtgccggcggatgacttcaccgatcccgccgtgaccaccatcgccgcccacgtggattccatggtggtgctctcccgcgccatggcggcggagggcatgtatccggcggtggaccccatctcctcctcgtcggtgctgctcgacccgctcatcgtgggggacgagcatgcgcgcgtcgccaacgaggtgcgccggaccatcgagcattatcgcgagcttcaggatgtgatctcgctgctgggcatggaggaattgggcaccgaggatcgccgcatcgtggagcgggcgcgccggctccagcgcttcctcacccagcccttcacggtcaccgaggccttcaccggcgtgcccggccgctcggtggccatcgccgacaccatcgccggctgcaggatgatcctgtccggcgcctgcgacgactggcaggaaagcgccctctacatggtgggcaccatcgacgaggcccgccagaaggaggaggccgctcgcgccaaggcggggcagggcgccccggccgggacggcagccgagacggcggaggccgccccgtga

SEQ ID NO: 31 - protein, 497 aa, Unknown
  novel strain
MAAADEEAQSAAGPASGRVVAVRGAVIDIAFAQPPLPPLDDALLITDGRGGTVLVEVQSHMDRHTVRAIALQATTGLSRGLEAARVGGPVKVPVGDHVLGRLLDVTGAIGDKGGPLPADVPTRPIHHAPPSFAAQGGTSDLFRTGIKVIDLLAPLAQGGKAAMFGGAGVGKTVLVMELIHAMVASYKGISVFAGVGERSREGHEMLLDMTDSGVLDRTVLVYGQMNEPPGARWRVPMTALTIAEYFRDEKHQNVLLLMDNIFRFVQAGAEVSGLLGRPPSRVGYQPTLASEVAALQERITSVGEASVTAIEAVYVPADDFTDPAVTTIAAHVDSMVVLSRAMAAEGMYPAVDPISSSSVLLDPLIVGDEHARVANEVRRTIEHYRELQDVISLLGMEELGTEDRRIVERARRLQRFLTQPFTVTEAFTGVPGRSVAIADTIAGCRMILSGACDDWQESALYMVGTIDEARQKEEAARAKAGQGAPAGTAAETAEAAP

SEQ ID NO: 32 - DNA, 1437 bp, Unknown
  novel strain
atggcgaacaaggtcggacgcatcacccagatcatcggcgccgtcgtcgacgtgcagttcgacgggcatctgccggcgattctcaacgcgatcgagaccaccaaccagggcaaccggctggtgctcgaagtggctcagcatctcggcgagaacaccgtgcgctgcatcgccatggatgccactgaaggcctggtgcgtggccaggaggtggccgacaccgatgcgcccatccaggtgcccgtgggcgccgccaccctcggccgcatcatgaacgtgatcggcgagccggtggacgagctgggccccatcgagggcgaagcgctgcgcggcatccatcagccggccccctcctatgcggagcaggccacggaagctgagatcctcgtcaccggcatcaaggtggtggatctgctggcgccctattccaagggcggcaaggtgggcctgttcggcggcgccggcgtgggcaagaccgtgctcatcatggagctgatcaacaacgtggccaaggcgcacggcggctattccgtgttcgccggcgtgggtgagcgcacccgcgagggcaacgacctctaccacgagatgatcgagtccaacgtgaacaaggacccgcacgagaacaatggctcggcggccggttccaagtgcgccctggtctatggccagatgaacgagccgcccggcgcccgcgcccgcgtggccctcaccggcctcaccgtcgccgagcatttccgcgaccagggccaggacgtgctgttcttcgtggacaacatcttccgcttcacccaggcgggctccgaggtgtcggcgcttctcggccgcatcccctcggcggtgggctaccagccgacgctggccaccgacatgggccagctgcaggagcgcatcaccaccaccaccaagggctccatcacctcggtgcaggccatctacgtgccggcggacgatctgaccgatccggcgccggccgcctccttcgcccatctggacgccaccacggtgctgtcgcgctccatcgcggagaagggcatctacccggcggtggatccgctggactccacctcgcgcatgctgtctcccgccatcctcggcgacgagcactacaacaccgcgcgccaggtgcagcagaccctgcagcgctacaaggcgctccaggacatcatcgccatcctgggcatggacgaactctccgaagaggacaagctcaccgtggcccgcgcccgcaagatcgagcgcttcctctcccagcccttccacgtggccgaggtgttcaccggttcgcccggcaagctggtcgacctcgccgacaccatcaagggcttcaagggcctggtggacggcaagtacgactacctgcccgagcaggccttctacatggtgggcaccatcgaagaagccatcgagaagggcaagaagctggcggccgaggcggcctga

SEQ ID NO: 33 - protein, 478 aa, Unknown
  novel strain
MANKVGRITQIIGAVVDVQFDGHLPAILNAIETTNQGNRLVLEVAQHLGENTVRCIAMDATEGLVRGQEVADTDAPIQVPVGAATLGRIMNVIGEPVDELGPIEGEALRGIHQPAPSYAEQATEAEILVTGIKVVDLLAPYSKGGKVGLFGGAGVGKTVLIMELINNVAKAHGGYSVFAGVGERTREGNDLYHEMIESNVNKDPHENNGSAAGSKCALVYGQMNEPPGARARVALTGLTVAEHFRDQGQDVLFFVDNIFRFTQAGSEVSALLGRIPSAVGYQPTLATDMGQLQERITTTTKGSITSVQAIYVPADDLTDPAPAASFAHLDATTVLSRSIAEKGIYPAVDPLDSTSRMLSPAILGDEHYNTARQVQQTLQRYKALQDIIAILGMDELSEEDKLTVARARKIERFLSQPFHVAEVFTGSPGKLVDLADTIKGFKGLVDGKYDYLPEQAFYMVGTIEEAIEKGKKLAAEAA

SEQ ID NO: 34 - DNA, 876 bp, Unknown
  novel strain
atggcgagtctgaaggacctgagaaaccgcattgcctcggtgaaggcgacgcagaagatcaccaaggcgatgcagatggtcgccgcggcgaagctgcgtcgcgcccaggcggcggctgaagcggcccgtccctatgcggaacgcatggagacggtgctcggaaatcttgcctccggcatggtggtgggcgcgcaggcgcctgttctcatgaccgggacgggcaagagcgacacccacctgctgctggtgtgcaccggcgagcgcggcctgtgcggcgccttcaactcgtccatcgtgcgcttcgcccgcgagcgggcgcagctgctgctggccgagggcaagaaggtgaaaatcctgtgcgtgggccgcaagggccacgagcagctgcgccgcatctacccggacaacatcatcgacgtggtggacctgcgcgcggtgcgcaacatcggcttcaaggaggccgacgccatcgcccgcaaggtgctggccctgctcgatgaaggcgcattcgacgtctgcacgctcttctactcccacttcaggagcgtgatcgcccaggtgccgacggcccagcagctcattccggccaccttcgacgagcggccggccgtcgccgatgcgccggtctatgaatatgagccggaggaggaggagatcctcgccgagctgctgccgcgcaacgtggcggtgcagatcttcaaggccctcctcgagaaccaggcttctttctatggctcccagatgagcgccatggacaacgccacgcgcaatgcgggcgagatgatcaagaagcagacgctcacctacaaccgtacccgccaggccatgatcacgaaggaactcatcgagatcatctccggcgccgaggccgtctga

SEQ ID NO: 35 - protein, 291 aa, Unknown
  novel strain
MASLKDLRNRIASVKATQKITKAMQMVAAAKLRRAQAAAEAARPYAERMETVLGNLASGMVVGAQAPVLMTGTGKSDTHLLLVCTGERGLCGAFNSSIVRFARERAQLLLAEGKKVKILCVGRKGHEQLRRIYPDNIIDVVDLRAVRNIGFKEADAIARKVLALLDEGAFDVCTLFYSHFRSVIAQVPTAQQLIPATFDERPAVADAPVYEYEPEEEEILAELLPRNVAVQIFKALLENQASFYGSQMSAMDNATRNAGEMIKKQTLTYNRTRQAMITKELIEIISGAEAV

SEQ ID NO: 36 - DNA, 1530 bp, Unknown
  novel strain
atggacattcgagccgctgaaatctctgccatcctgaaagagcagatccagaatttcggccaggaggcggaagtctccgaggtgggtcaggttctgtccgtgggtgacggcatcgcgcgcgtctacggcctcgacaacgtccaggcgggcgagatggtcgagttcgagaacggcacgcgcggcatggcgctgaacctcgagctcgacaatgtcggcatcgtgatcttcggttccgaccgcgagatcaaggaaggccagaccgtcaagcggaccggcgccatcgtggacgcccccgtcggcaagggcctgctcggccgcgtcgtggacgctctcggcaacccgatcgacggcaagggcccgatcatgttcaccgagcgtcgccgggtcgacgtgaaggcgccgggcatcatcccgcgcaagtcggtgcacgagcccatgcagaccggcctgaaggccatcgatgcgctcatccccatcggccgcggccagcgcgagctcatcatcggcgaccgccagaccggcaagaccgccgtggcgctcgactcgatcctgaaccagaagcccatcaaccagggcgacgacgagaaggccaagctctactgcgtctatgtcgcggtgggccagaagcgttccactgtcgcgcagttcgtgaaggtgctcgaggagcacggcgcgctggaatattccatcgtcgtcgccgccaccgcctcggacgcggcccccatgcagttcctggcgccgttcaccggcaccgccatgggcgagtatttccgcgacaacggcatgcacgccctcatcatccatgatgacctgtccaagcaggccgtggcctaccgccagatgtcgctgctgctgcgccgcccgccgggccgcgaggcctatcccggcgatgtgttctacctgcactcccgcctcttggagcgcgccgccaagctcaatgacgagcacggcgccggctcgctgaccgccctgccggtgatcgagacccaggccaacgacgtgtcggcctacatcccgaccaacgtgatctccatcaccgacggtcagatcttccttgaatccgatctgttctaccagggcatccgcccggcggtgaacgtgggcctgtcggtgtcgcgcgtgggctcttcggcccagatcaaggcgatgaagcaggtggccggcaagatcaagggcgagctcgcccagtatcgcgagctggcggccttcgcccagttcggttcggacctggacgcggccacccagaagctgctgaaccgcggcgcccgcctcaccgagctgctgaagcagagccagttctcgcccctcaaggtggaggagcaggtggcggtgatctatgccggcaccaatggctatctcgatccgctgccggtctccaaggtgcgcgagttcgagcagggtctgctcctgtcgctgcgctcgcagcatccggagatcctggacgccatccgcacgtccaaggagctttccaaggacaccgccgagaagctgacgaaggccatcgacgccttcgccaagagcttctcctga

SEQ ID NO: 37 - protein, 509 aa, Unknown
  novel strain
MDIRAAEISAILKEQIQNFGQEAEVSEVGQVLSVGDGIARVYGLDNVQAGEMVEFENGTRGMALNLELDNVGIVIFGSDREIKEGQTVKRTGAIVDAPVGKGLLGRVVDALGNPIDGKGPIMFTERRRVDVKAPGIIPRKSVHEPMQTGLKAIDALIPIGRGQRELIIGDRQTGKTAVALDSILNQKPINQGDDEKAKLYCVYVAVGQKRSTVAQFVKVLEEHGALEYSIVVAATASDAAPMQFLAPFTGTAMGEYFRDNGMHALIIHDDLSKQAVAYRQMSLLLRRPPGREAYPGDVFYLHSRLLERAAKLNDEHGAGSLTALPVIETQANDVSAYIPTNVISITDGQIFLESDLFYQGIRPAVNVGLSVSRVGSSAQIKAMKQVAGKIKGELAQYRELAAFAQFGSDLDAATQKLLNRGARLTELLKQSQFSPLKVEEQVAVIYAGTNGYLDPLPVSKVREFEQGLLLSLRSQHPEILDAIRTSKELSKDTAEKLTKAIDAFAKSFS

SEQ ID NO: 38 - DNA, 555 bp, Unknown
  novel strain
gtggcggaaacgatcgtgtcaggcatggcgggacgctatgcgaccgcgctgttcgagctggcggacgaagccggtgccatcgattccgtccaggcggatcttgatcgcctgtccggccttctggccgagagcgcggatctggcgcggctggtcaagagcccggtcttcaccgccgagcagcagctcggcgcgatggcggccattctcgatcaagcaggcatttccggccttgcgggcaaattcgtgaagctggtggcgcagaaccgccgcctgttcgcactgccgcgcatgattgccgaatacgccgtcctggtggcccggaagaagggcgagacctcggcgagcgtgaccgttgccacccccctgagcgatgagcatctggccacgctcaaggcggccctggctgaaaagaccggcaaggacgtgaagctcgacgtcaccgtcgatccgtccatcctcggtggtctcatcgtgaagctcggctcgcgcatggtcgatgcttccctgaagaccaaactcaattctatccggcatgcgatgaaagaggtccgctga

SEQ ID NO: 39 - protein, 184 aa, Unknown
  novel strain
MAETIVSGMAGRYATALFELADEAGAIDSVQADLDRLSGLLAESADLARLVKSPVFTAEQQLGAMAAILDQAGISGLAGKFVKLVAQNRRLFALPRMIAEYAVLVARKKGETSASVTVATPLSDEHLATLKAALAEKTGKDVKLDVTVDPSILGGLIVKLGSRMVDASLKTKLNSIRHAMKEVR

SEQ ID NO: 40 - DNA, 492 bp, Unknown
  novel strain
atgaccgaaatggaactggctgagctctgggtcgccatcgccttcctggttttcgtaggcctcctgatctatgcgggcgcccaccgcgccatcgtctccgccctggattcccgcggctcgcgcatcgcctcggaactggaggaggcccgtcggctcaaggaagaggcccagaagctggtggccgaattcaagcgcaagcagcgcgaggccgaggccgaggccgaatccatcgtcaccggcgccaaggccgaggccgagcgcctcgccgccgaggccaaggcgaagatcgaggatttcgtcacccgccgcaccaagatggccgaggacaagatcgcccaggccgagcatcaggctctggcggacgtgaagtccatcgccgccgaggcggcggccaaggcggccgaggtgatcctcggcgcccaggccaccggcgcggtggcggagcgtctgctgtcgggcgccatctccgaggtcaagaccaagctcaactga

SEQ ID NO: 41 - protein, 163 aa, Unknown
  novel strain
MTEMELAELWVAIAFLVFVGLLIYAGAHRAIVSALDSRGSRIASELEEARRLKEEAQKLVAEFKRKQREAEAEAESIVTGAKAEAERLAAEAKAKIEDFVTRRTKMAEDKIAQAEHQALADVKSIAAEAAAKAAEVILGAQATGAVAERLLSGAISEVKTKLN

SEQ ID NO: 42 - DNA, 729 bp, Unknown
  novel strain
atgatgattgcatggaagcggaccttcgcagtcgtgaccttcggggccgccctgatggccatgcccgtcgcgggcgtggtcgcagctgagacttctcccgctccggcggcagtggcgcaggccgatcatgcggtgcccaccgaggcggccggccagggcaccgccgatgcggcccatgccgccgcgccgggcgaggccgcccatggtggcgcggccaagcacgaaacccatttcccgcccttcgacggcaccaccttcgcctcccagttgctgtggctcgccgtcaccttcggcctgctttactacctcatgagcaaggtcacgctgccgcgcatcggccgcatcctggaagagcgccacgaccgcatcgccgatgatctggaggaagcctccaagcatcgcgccgagagcgaggccgcccagcgggcctatgagaaggcgctgagcgaggcccgcgcgaaggcccattccatcgccgcggaaacccgcgaccgccttgccgcccacgccgacaccaaccgcaaggcgctggagagcgagctcaccgccaagctgcaggcggccgaggagcgcatcgccaccaccaagagcgaagccctcacccatgtgcgcggcatcgcggtggacgccacccaatccatcgtctccaccctcatcggtgtcgcgcccgcggcggccgacgtggaaaaagcggtggacggcgccctgtcccagcacggccaggcctga

SEQ ID NO: 43 - protein, 242 aa, Unknown
  novel strain
MMIAWKRTFAVVTFGAALMAMPVAGVVAAETSPAPAAVAQADHAVPTEAAGQGTADAAHAAAPGEAAHGGAAKHETHFPPFDGTTFASQLLWLAVTFGLLYYLMSKVTLPRIGRILEERHDRIADDLEEASKHRAESEAAQRAYEKALSEARAKAHSIAAETRDRLAAHADTNRKALESELTAKLQAAEERIATTKSEALTHVRGIAVDATQSIVSTLIGVAPAAADVEKAVDGALSQHGQA

SEQ ID NO: 44 - DNA, 228 bp, Unknown
  novel strain
atggaagcggaagctggaaagttcatcggtgccggcctcgcctgcctcggcatgggtctcgctggcgtcggcgtcggtaacatcttcggtaacttcctctccggcgccctgcgcaacccgtccgctgccgacggccagttcgcccgcgccttcatcggcgccgccctcgcggaaggtctcggcatcttctcgctggtcgttgcgctcgtcctgctgttcgtggcctga

SEQ ID NO: 45 - protein, 75 aa, Unknown
  novel strain
MEAEAGKFIGAGLACLGMGLAGVGVGNIFGNFLSGALRNPSAADGQFARAFIGAALAEGLGIFSLVVALVLLFVA

SEQ ID NO: 46 - DNA, 753 bp, Unknown
  novel strain
atgaccgtcgatccgatccaccagttcgagatcaagcgctacgtggatctgctgaacgtcggcggtgtccagttctccttcaccaacgcaacggtgttcatgattggcatcgtcctggtgattttcttcttcctgactttcgcgacacgcggtcgcacccttgtgccgggccggatgcagtcggcggcggagctgagctacgagttcatcgccaagatggtgcgcgacgcggccggcagcgagggaatggtgttctttcccttcgtcttctcgctcttcatgttcgtgctggtggcgaacgtattggggctcatcccctacaccttcacggtgaccgcccacctcatcgtcaccgccgccctggcggcgacggtgatcctcaccgtcatcatctacggcttcgtgcggcacggcacccacttcctgcacctgttcgtgccgtcgggcgtgccgggcttcctcctgcccttcctcgtggtgatcgaggtggtgtcgttcctgtcgcggcccatcagcctctcgctgcgtctgttcgccaacatgctggcgggccacatcgccctcaaggtgttcgccttcttcgtcgtgggactggcctcggccggcgcgatcggctggttcggcgccaccctgcccttcttcatgatcgtggcgctcaccgcgctggagctgctggtggcggtgctgcaggcctacgtgttcgcggtgctgacctcgatctacctcaacgacgccatccatcccggccactga

SEQ ID NO: 47 - protein, 250 aa, Unknown
  novel strain
MTVDPIHQFEIKRYVDLLNVGGVQFSFTNATVFMIGIVLVIFFFLTFATRGRTLVPGRMQSAAELSYEFIAKMVRDAAGSEGMVFFPFVFSLFMFVLVANVLGLIPYTFTVTAHLIVTAALAATVILTVIIYGFVRHGTHFLHLFVPSGVPGFLLPFLVVIEVVSFLSRPISLSLRLFANMLAGHIALKVFAFFVVGLASAGAIGWFGATLPFFMIVALTALELLVAVLQAYVFAVLTSIYLNDAIHPGH

SEQ ID NO: 48 - DNA, 390 bp, Unknown
  novel strain
atgtccgagccgaatgatccatcccgcagggacggtgcgaaggcgaaagacgagacgcaggactcccggcccggtgaggcggatcttgctcggcgcctcgatgcgctcggcacctccatcggtcaggtcaagtccagaagcggggagcccgcggcgacgccgcgcaaggacacctcctcggcctccggcgcggccctggcgtttcggctgggcgccgagtttgtttcaggcgtgctggtgggctcgctcatcggctacgggttggattatgcgtttgcgatttcgccctgggggctgatcgccttcacgctgatcggctttgccgccggcgtcctgaacatgctgcgcgtggcgaacagcgatgccaagcgccacagcgcggacaggtga

SEQ ID NO: 49 - protein, 129 aa, Unknown
  novel strain
MSEPNDPSRRDGAKAKDETQDSRPGEADLARRLDALGTSIGQVKSRSGEPAATPRKDTSSASGAALAFRLGAEFVSGVLVGSLIGYGLDYAFAISPWGLIAFTLIGFAAGVLNMLRVANSDAKRHSADR

SEQ ID NO: 50 - DNA, 1929 bp, Unknown
  novel strain
atgagttcgctctccgccactattcaacaggtcttcaacgagccgggctgcgcgaagaaccagaataagtccgaggcggagaagaagaagggctgcaccaagcagctgcaacccggcggagcggccggcggctgcgcgttcgacggcgcgaagatcgcgctccagcccttgaccgacgtcgcccacctggtgcacggccccatcgcctgcgaaggcaattcctgggacaatcgtggcgccaagtcctccggctcgaacatctggcgcaccggcttcaccacggacatcaacgaaaccgacgtggtgttcggcggcgagaagcgtctgttcaagtccatcaaggaaatcatcgagaagtacgacccgccggccgtcttcgtctatcagacctgcgtccccgccatgatcggcgacgacatcgacgcggtgtgcaaggcggccagggagaagttcggaaagccggtgatcccgatcaattcccccggcttcgtggggccgaagaatctcggcaacaagctcgccggcgaggcgctcctcgaccatgtgatcggcaccgaggagcccgattacacgacggcctacgacatcaacatcatcggcgaatacaatctctccggcgagttgtggcaggtgaagccgctgctggacgagctgggcatccgcatcctcgcctgcatctccggcgacgggaagtacaaggatgtggcgtcctcccaccgcgccaaggcggcgatgatggtgtgctccaaggccatgatcaacgtggcccgcaagatggaggagcgctacgacatccccttcttcgaaggctccttctacggcatcgaggatagctccgattccctgcgcgagattgcgcgcatgctcatcgagaagggcgccgatccggagctgatggaccgcaccgaggcgctgattgagcgggaagagaagaaggcgtgggacgccatcgccgcctacaagccccgcttcaaggacaagaaggtgctgctcatcaccggcggcgtgaaatcctggtcggtggtggcagcgctccaggaagccggcctcgaactggtgggcacctcggtgaagaagtccaccaaggaggacaaggagcgcatcaaggaactgatgggccaggacgcccacatgatcgacgacatgacgccccgcgaaatgtacaagatgctgaaggacgccaaggcggacatcatgctctcgggcgggcgctcgcaattcatcgcgctcaaggccgccatgccctggctcgacatcaaccaggagcgccaccacgcctatatgggctatgtgggcatggtgaagctggtcgaggagatcgacaaggcgctctacaatcccgtgtgggaacaggtgcgcaagcccgccccgtgggaaaatccggaagacacctggcaggcccgtgcgctcgccgaaatggaggcggaggccgccgcgctcgccgccgatccggtgcgcgcggaagaggtgcgccggtccaagaagatctgcaattgcaagagcgtcgacctcggaaccattgaggacgccatcaaggctcacgcgctgaccaccgtggagggtgtgcgagagcacaccaatgcctcgggaggctgcggagcctgcagcgggcggatcgaggagatcttcgaggccgtgggcgttgtcgccgccccgcctcccgcggaggccgccccgtctccgcaggagatcgcgcccgatccgctcgctgcggaggaaaagcgccgcgccaagaaggcctgcggctgcaaggaggtagcggtcggcaccattgaggatgccatccgcgccaagggtctgcgaaacatcgcggaggtgcgtgcggccaccgatgccaacaccggctgcggcaattgccaggagcgggtggagggcatcctcgaccgggttctcgccgaggcggcctcagaactccaggcggcggaatag

SEQ ID NO: 51 - protein, 642 aa, Unknown
  novel strain
MSSLSATIQQVFNEPGCAKNQNKSEAEKKKGCTKQLQPGGAAGGCAFDGAKIALQPLTDVAHLVHGPIACEGNSWDNRGAKSSGSNIWRTGFTTDINETDVVFGGEKRLFKSIKEIIEKYDPPAVFVYQTCVPAMIGDDIDAVCKAAREKFGKPVIPINSPGFVGPKNLGNKLAGEALLDHVIGTEEPDYTTAYDINIIGEYNLSGELWQVKPLLDELGIRILACISGDGKYKDVASSHRAKAAMMVCSKAMINVARKMEERYDIPFFEGSFYGIEDSSDSLREIARMLIEKGADPELMDRTEALIEREEKKAWDAIAAYKPRFKDKKVLLITGGVKSWSVVAALQEAGLELVGTSVKKSTKEDKERIKELMGQDAHMIDDMTPREMYKMLKDAKADIMLSGGRSQFIALKAAMPWLDINQERHHAYMGYVGMVKLVEEIDKALYNPVWEQVRKPAPWENPEDTWQARALAEMEAEAAALAADPVRAEEVRRSKKICNCKSVDLGTIEDAIKAHALTTVEGVREHTNASGGCGACSGRIEEIFEAVGVVAAPPPAEAAPSPQEIAPDPLAAEEKRRAKKACGCKEVAVGTIEDAIRAKGLRNIAEVRAATDANTGCGNCQERVEGILDRVLAEAASELQAAE

SEQ ID NO: 52 - DNA, 1503 bp, Unknown
  novel strain
atgagtgtcgcacagtcccagagcgtcgccgagatcaaggcgcgcaacaaggaactcatcgaagaggtcctcaaggtctatcccgagaagaccgccaagcgccgcgccaagcacctgaacgtccacgaagccggcaagtccgactgcggcgtgaagtccaacatcaagtccatcccgggcgtgatgaccatccgcggttgcgcttatgccggctccaagggtgtggtgtggggtcccatcaaggacatgatccacatctcccacggcccggtgggctgcggccagtatagctgggccgcccgccgcaactactatatcggcacgaccggcatcgacaccttcgtgacgatgcagttcacctccgacttccaggagaaggacatcgtcttcggcggcgacaagaagctcgccaagatcatggacgagatccaggagctgttcccgctgaacaacggcatcaccgttcagtccgagtgccccatcggcctcatcggcgacgacatcgaggccgtctccaagcagaagtccaaggagtatgagggcaagaccatcgtgccggtgcgctgcgagggcttccgcggcgtgtcccagtccctgggccaccacatcgccaacgacgccatccgcgattgggtgttcgacaagatcgcgcccgacgccgagccgcgctttgagccgaccccgtacgacgtcgccatcatcggcgactacaatatcggtggtgacgcctggtcgtcccgtatcctcctggaggagatgggcctgcgcgtgatcgcccagtggtccggcgacggttcgctcgctgagctggaggccaccccgaaggccaagctcaacgtgctgcactgctaccgctccatgaactacatctcgcgccacatggaagagaagtacggtatcccgtggtgcgagtacaacttcttcggtccttccaagatcgccgagtccctgcgcaagatcgccagctacttcgacgacaagatcaaggaaggcgcggagcgcgtcatcgccaagtatcagccgctcatggatgcggtgatcgcgaagtatcgtccccgcctcgagggcaagaccgtgatgctgtacgtgggcggcctgcgtccccgtcacgtcatcggcgcctacgaggacctgggcatggaagtggtcggcacgggctacgagttcgcccataacgacgactaccagcgcaccgcccagcactacgtcaaggatggcaccatcatctatgacgacgtgaccggctacgagttcgagaagttcgtcgagaagatccagccggacctggtcggttcgggcatcaaggaaaagtacgtcttccagaagatgggcgtgccgttccgccagatgcactcctgggactactcgggcccgtaccacggctatgacggcttcgcgatcttcgcgcgcgacatggacatggccatcaacagccccgtgtggaagatgacccaggctccgtggaagagcgtccccaagccgacgatgctcgcggctgaatga

SEQ ID NO: 53 - protein, 500 aa, Unknown
  novel strain
MSVAQSQSVAEIKARNKELIEEVLKVYPEKTAKRRAKHLNVHEAGKSDCGVKSNIKSIPGVMTIRGCAYAGSKGVVWGPIKDMIHISHGPVGCGQYSWAARRNYYIGTTGIDTFVTMQFTSDFQEKDIVFGGDKKLAKIMDEIQELFPLNNGITVQSECPIGLIGDDIEAVSKQKSKEYEGKTIVPVRCEGFRGVSQSLGHHIANDAIRDWVFDKIAPDAEPRFEPTPYDVAIIGDYNIGGDAWSSRILLEEMGLRVIAQWSGDGSLAELEATPKAKLNVLHCYRSMNYISRHMEEKYGIPWCEYNFFGPSKIAESLRKIASYFDDKIKEGAERVIAKYQPLMDAVIAKYRPRLEGKTVMLYVGGLRPRHVIGAYEDLGMEVVGTGYEFAHNDDYQRTAQHYVKDGTIIYDDVTGYEFEKFVEKIQPDLVGSGIKEKYVFQKMGVPFRQMHSWDYSGPYHGYDGFAIFARDMDMAINSPVWKMTQAPWKSVPKPTMLAAE

SEQ ID NO: 54 - DNA, 1377 bp, Unknown
  novel strain
atggccaccgtttccgtctccaagaaggcctgcgcggtcaaccccctcaagatgagccagccggtgggcggcgcgctcgccttcatgggcgtgcgcaaggccatgccgctgctgcacggctcgcagggctgcacctccttcggcctggtgctgttcgtgcgccacttcaaggaagccatccccatgcagaccaccgccatgagcgaggtggcgacggttctgggcggccttgagaatgtggagcaggccattctcaacatctacaatcgcaccaagccggagatcatcggcatctgctccaccggcgtcaccgagaccaagggcgatgatgtcgacggctacatcaagctgatccgggacaagtatccccagctggccgacttcccgctggtctatgtctccacccccgatttcaaggacgccttccaggacggttgggagaagaccgtggcgaagatggtggaggcgctggtgaagcccgccgccgacaagcagaaggacaagacccgcgtcaacgtcctgcccggctgccacctcacgcccggcgatctggatgagatgcggaccatcttcgaggatttcgggctcacaccctatttcctgccggatctggccggctcgctggatgggcatatccccgaggacttctcgcccaccaccatcggcggcatcggcatcgatgagatcgccaccatgggcgaggcggcccacaccatctgcatcggcgcgcagatgcgccgggcgggcgaggccatggagaagaagaccggcattcccttcaagctgttcgagcgcctgtgcggcctggaggcgaacgacgccttcatcatgcacctgtcgcagatctccggccggccggtgccggtgaagtatcgccggcagcggggccagctggtggatgccatgctggacggccacttccatctgggcggtcgcaaggtggccatgggggcggagccggacctgctctacgacgtgggctccttcctgcacgagatgggcgcccacatcctttccgcggtcaccaccacccagtcgccggtgctggcgcgcctgcctgccgaggaggtgcttatcggcgacctggaggatctggagacccaggcgaaggcgcgcggatgcgatctcctgctcacccattcccatgggcgccaggcggcggagcgcctccacatccccttctaccggatcggcattcccatgtttgaccggctgggggcggggcatctgttgtcggtgggctatcgcggcacccgcgacctcatcttccatctcgccaaccttgtgatcgccgaccacgaggaaaatcacgagccgacgcccgacacctgggccaccggccatggcgagcatgccgccgcccccacttcccattga

SEQ ID NO: 55 - protein, 458 aa, Unknown
  novel strain
MATVSVSKKACAVNPLKMSQPVGGALAFMGVRKAMPLLHGSQGCTSFGLVLFVRHFKEAIPMQTTAMSEVATVLGGLENVEQAILNIYNRTKPEIIGICSTGVTETKGDDVDGYIKLIRDKYPQLADFPLVYVSTPDFKDAFQDGWEKTVAKMVEALVKPAADKQKDKTRVNVLPGCHLTPGDLDEMRTIFEDFGLTPYFLPDLAGSLDGHIPEDFSPTTIGGIGIDEIATMGEAAHTICIGAQMRRAGEAMEKKTGIPFKLFERLCGLEANDAFIMHLSQISGRPVPVKYRRQRGQLVDAMLDGHFHLGGRKVAMGAEPDLLYDVGSFLHEMGAHILSAVTTTQSPVLARLPAEEVLIGDLEDLETQAKARGCDLLLTHSHGRQAAERLHIPFYRIGIPMFDRLGAGHLLSVGYRGTRDLIFHLANLVIADHEENHEPTPDTWATGHGEHAAAPTSH

SEQ ID NO: 56 - DNA, 1560 bp, Unknown
  novel strain
atgccacaaaatgctgacaatgtgctcgatcacttcgagctcttccgtggtcccgaataccagcagatgctggccaataagaaaaagatgttcgagaacccccgcgatccggccgaagtcgagcgcgtgcgggaatgggcgaagactcctgaatacaaggagctgaacttcgcccgcgaggcgctcaccgtgaatccggccaaggcttgtcagccgctgggcgcggtgttcgtcgccgtcggcttcgagagcacgatccccttcgtgcacggctcgcagggttgcgtcgcgtattaccgctcgcacctctcccgccacttcaaggagccgtcctcctgcgtctcctcgtccatgaccgaggatgcggcggtgttcggcggcctcaacaacatgattgacggcctcgccaacacctacaacatgtacaagccgaagatgatcgccgtctccaccacctgcatggcggaagtcatcggcgacgatctgaacgccttcatcaagaccgcgaaggaaaagggctcggttccggccgaatacgacgtgcccttcgcccacaccccggcgttcgtcggcagccatgtcaccggctacgacaatgcgctcaagggcatcctcgagcacttctgggacggcaaggccggcaccgcgccgaagctggagcgcgttcccaacgagaagatcaacttcatcggcggcttcgacggctacaccgtcggcaacactcgcgaagtgaagcgcatcttcgaggcgttcggcgccgattacaccatcctcgccgacaattccgaagtgttcgacaccccgaccgacggcgagttccgcatgtatgacggcggcacgaccctggaggacgcggcgaacgcggtgcacgccaaggccaccatctccatgcaggaatactgcacggagaagaccctgcccatgatcgccggtcatggccaggacgtggtcgccctcaaccaccccgtgggcgtgggcggcaccgacaagttcctcatggagatcgcccgcctcaccggcaaggagatccccgaggagctgacccgcgagcgcggccgtctcgtggacgctatcgcggactcttccgcgcacatccacggcaagaagttcgccatctacggcgatccggatctgtgcctgggcctcgccgcgttcctgctggagctgggcgccgagccgacccatgtgctggccaccaacggcaccaagaagtgggccgagaaggttcaggaactgttcgactcttcgccgttcggcgccaactgcaaggtctatcccggcaaggacctgtggcacatgcgctcgctcctgttcgtggagccggtggatttcatcatcggcaacacctacggcaagtatctcgagcgcgacacgggcaccccgctgatccgtatcggcttcccggtgttcgaccgtcaccaccaccaccgccgtccggtgtggggctatcagggcggcatgaacgtcctgatcacgatcctcgacaagatctttgacgagatcgaccgcaacaccaacgtgccggccaagaccgactactcgttcgacatcattcgttga

SEQ ID NO: 57 - protein, 519 aa, Unknown
  novel strain
MPQNADNVLDHFELFRGPEYQQMLANKKKMFENPRDPAEVERVREWAKTPEYKELNFAREALTVNPAKACQPLGAVFVAVGFESTIPFVHGSQGCVAYYRSHLSRHFKEPSSCVSSSMTEDAAVFGGLNNMIDGLANTYNMYKPKMIAVSTTCMAEVIGDDLNAFIKTAKEKGSVPAEYDVPFAHTPAFVGSHVTGYDNALKGILEHFWDGKAGTAPKLERVPNEKINFIGGFDGYTVGNTREVKRIFEAFGADYTILADNSEVFDTPTDGEFRMYDGGTTLEDAANAVHAKATISMQEYCTEKTLPMIAGHGQDVVALNHPVGVGGTDKFLMEIARLTGKEIPEELTRERGRLVDAIADSSAHIHGKKFAIYGDPDLCLGLAAFLLELGAEPTHVLATNGTKKWAEKVQELFDSSPFGANCKVYPGKDLWHMRSLLFVEPVDFIIGNTYGKYLERDTGTPLIRIGFPVFDRHHHHRRPVWGYQGGMNVLITILDKIFDEIDRNTNVPAKTDYSFDIIR

SEQ ID NO: 58 - DNA, 621 bp, Unknown
  novel strain
gtggagtccggtggtcctgagccgggcgtgggctgcgccggccgcggcgtgatcacctccatcaacttcctggaggagaacggcgcctacgaggacatcgactatgtgtcctacgacgtgctgggcgacgtggtgtgcggcggcttcgccatgcccatccgcgagaacaaggcgcaggaaatctacatcgtgatgtccggcgagatgatggccatgtatgcggccaacaacatctccaagggcatcctgaagtatgccaattccggcggcgtgcgcctgggcgggctggtctgcaacgagcgccagaccgacaaggagctggagctggcggaggctctggcgaagaagctcggcaccgagctgatctacttcgtgccgcgcgacaacatcgtgcagcatgccgagctgcgccgcatgacagtgatcgagtatgcgcccgattccgcccaggcccagcactaccggaacctggccgagaaggtgcacgccaacaagggcaacggcatcatcccgaccccgatcaccatggacgagctggaagacatgctcatggagcacggcatcatgaaggccgtggacgagagccagatcggcaagaccgccgccgagctcgccgtctga

SEQ ID NO: 59 - protein, 206 aa, Unknown
  novel strain
MESGGPEPGVGCAGRGVITSINFLEENGAYEDIDYVSYDVLGDVVCGGFAMPIRENKAQEIYIVMSGEMMAMYAANNISKGILKYANSGGVRLGGLVCNERQTDKELELAEALAKKLGTELIYFVPRDNIVQHAELRRMTVIEYAPDSAQAQHYRNLAEKVHANKGNGIIPTPITMDELEDMLMEHGIMKAVDESQIGKTAAELAV